(19) United States
(12) Patent Application Publication
(10) Pub. No.: US 2007/0093476 A1
(43) Pub. Date: Apr. 26, 2007

(54) Novel compounds and their use in medicine,as antidiabetic and hypolipidemic agents, process for their preparation and pharmaceutical compositions containing them

(76) Inventors: Bhunia Debnath, Hyderabad (IN); Ranga Madhavan Gurram, Hyderabad (IN); Das Saibal Kumar, Hyderabad (IN); Iqbal Javed, Hyderabad (IN); Chakrabarti Ranjan, Hyderabad (IN); Kole Labanyamoy, Hyderabad (IN)

Correspondence Address: Milagros A Cepeda;Reddy's Laboratories Inc, 200 Somerset Corporate Blvd, Seventh Floor, Bridgewater, NJ, 08807-2862, US

(21) Appl. No.: 10/577,698

(22) PCT Filed: Oct. 20, 2004
(86) PCT No.: PCT/IB04/03429

§ 371 Date: Apr. 28, 2006

(30) Foreign Application Priority Data
Oct. 28, 2003 (IN) 862/CHE/2003

(51) Int. Cl. A61K 31/5415; A61K 31/538; A61K 31/4704; A61K 31/404; A61K 31/195

(52) U.S. Cl. 514/224.2; 514/230.5; 514/309; 514/312; 514/418; 514/567; 514/562; 514/563; 544/105; 546/144; 546/153; 548/484; 544/51; 562/433; 562/427; 514/417

(57) ABSTRACT

The present invention relates to novel compounds of formula (I) and their pharmaceutically acceptable salts, wherein ring “Ar₁” represents a monocyclic or polycyclic aromatic or partially saturated aromatic polycyclic, which may optionally contain up to 3 heteroatoms selected from N, S or O. The said monocyclic or polycyclic ring may be unsubstituted or have up to 4 substituents which may be identical or different; m and n independently represents an integer from 0 to 6; A represents O, S or bond; Y is selected from (CH₂)_{p′}(CH₂)ₚB(CH₂)_{q′}(CH₂)ᵣB(CH₂)ₚD(CH₂)_{p′} where p, q and r each independently represents an integer from 0 to 6; B and D independently represents S, O, NR⁴ or a bond, with a proviso that when B and D represents hereto atom p is not zero; R⁴ represents hydrogen, alkyl, alkenyl, —S(O)₂—R⁸ or —C(O)R⁸ where R⁸ is alkyl, alkoxy; R⁵ and R⁶ independently represents hydrogen, alkyl, cycloalkyl or alkoxy; R⁵ and R⁶ together may form 3-8 membered cyclic ring which may optionally contains one or two hereto atoms selected from O, S or N; R⁷ represents hydrogen, optionally substituted groups selected form alkyl, cycloalkyl, alkenyl or alkynyl. The present invention also relates to a process for preparation of compounds of formula (I), to pharmaceutical compositions containing compounds of formula (I) and their use in particular as antidiabetic, hypolipidemic, antiobesity and hypocholesterolemic agents.

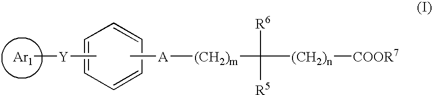

### FIELD OF THE INVENTION

The present invention relates to the novel compounds of formula (I) and their pharmaceutically acceptable salts. The present invention also relates to a process for the preparation of compounds of formula (I), to pharmaceutical compositions containing compounds of formula (I) and their use in particular as antidiabetic, hypolipidemic, antiobesity and hypocholesterolemic agents.

The compounds of the present invention lower plasma glucose, triglycerides, lower total cholesterol (TC) and increase high density lipoprotein (HDL) and decrease low density lipoprotein (LDL), which have a beneficial effect on in cardio vascular disease like coronary heart disease and atherosclerosis.

### DESCRIPTION OF RELATED ART

Peroxisome Proliferator Activated Receptors (PPARs) are orphan receptors belonging to the steroid/retinoid receptor super family of ligand activated transcription factors. (Wilson T. M. and Wahli W., Curr. Opin. Chem. Biol., 1997, Vol. 1, 235-241). Three mammalian Peroxisome Proliferator Activated Receptors (PPARs) have been isolated and termed PPAR-α, PPAR-γ and PPAR-δ. These PPARs regulate expression of target genes by binding to DNA sequence elements. Certain compounds that activate or otherwise interact with one or more of the PPARs have been implicated in the regulation of triglyceride and cholesterol levels in animal models. (U.S. Pat. Nos. 5,847,008; 5,859,051 and PCT publications WO 97/28149; WO 99/04815.

Weak PPARα agonists such as fibrate class of compounds correct atherogenic dyslipoproteinemia. Several angiographic intervention trials have demonstrated a beneficial action of these drugs on atherosclerotic lesion progression and results from primary and secondary prevention trials show a decreased incidence of cardiovascular events. (Ricote M. and Glass C. K.; Trends in Pharmacological Sciences; 2001; 22(9); 441-443.

Despite the fact that fibrates, which are weak PPAR-α activators, reduce the plasma triglyceride levels and elevate the levels of HDL-C simultaneously, they are not the drugs of choice, because of: low efficacy requiring high doses, incidence of Myositis and contra-indicated in patients with impaired renal and hepatic function and to pregnant and nursing women.

However there has been rapid progress in our understanding on the role of PPAR-α in different pathophysiological conditions in addition to the well-documented favourable effects on lipid profile. The inflammatory activation of aortic smooth muscle cells, which is the hallmark of atherosclerosis, seems to be inhibited by the enhanced PPAR-α activity. (Vamecq J. and Latruffe N; Lancet; 1999; 354; 141-148)

Recent evidence suggests the role of PPAR-α receptors in improving insulin sensitivity. It has been demonstrated that by lowering circulatory and muscle lipids in insulin-resistant rodent models such as obese Zuker rats, high fat-fed mice and sucrose-lard fed rats, PPAR-α ligands improve insulin sensitivity and obesity. Further the lipid lowering activity of the statins has been linked to a cross talk with PPAR-α receptor in addition to limited cholesterol availability. Some clinical trials have shown improvement in insulin sensitivity indices, wherein fibrates were employed. (i. Guerre-Millo M, Rounalt C. and Poulain P; Diabetes; 2001; 50; 2809-2814, ii. Muoio D. M., Way J. M. and Tanner C. J.; Diabetes; 2002; 51; 901-909, iii. Ye J, Doyle P. J. and Iglesias M. A.; Diabetes; 2001; 50; 411-417, iv. Roglans N, Sanguino E. and Peris C; JPET; 2002; 302; 232-239.

Thus there is an interesting evidence for PPAR-α agonists to be used for lipid control and as per recent evidence even for insulin resistance. Limitations of the currently available medications coupled with the fact that lipid abnormalities are on the rise world over necessitate the discovery of more potent and safer PPAR-α agonists. In continuation of our research work on PPAR agonists (U.S. Pat. Nos. 5,885,997; 6,054,453; 6,265,401: PCT application PCT/IB02/04275) to address this unmet need, a series of compounds have been synthesized which has been disclosed in the present invention.

The patent application WO 98/31359 describes substituted aromatic or non aromatic ring systems as vitronectin receptor antagonists. GB 2202223 describes sulfonylcarboxamides for the treatment of leukotriene-mediated naso-bronchial obstructive airpassageway conditions. U.S. Pat. Nos. 600,117 and 6,399,620 describes imino derivatives as vitronectin receptor antagonists and also as inhibitors of bone resorption. GB 2310669 describes substituted aromatic or non aromatic ring systems as a liquid crystalline medium. WO 92/01675 describes substituted bicyclic bis-aryl compounds which exhibit selective leukotriene B₄ antagonist activity. WO 01/53257 describes substituted pyrrole derivatives having hypolipedemic, hypocholesteremic activities.

### SUMMARY OF THE INVENTION

The present invention is directed to novel compounds, their pharmaceutically acceptable salts capable of being used as antidiabetic, hypolipidemic, antiobesity and hypocholesterolemic agents.

The present invention also directed to methods for the production of the compounds of the present invention. The present invention also directed pharmaceutical composition which includes the compound of the present invention. The present invention is directed to methods for the treating diabetes, dyslipidemia, hypercholesterolemia, obesity and hypertriglyceridemia.

One aspect of this invention provides novel compounds of formula (I) 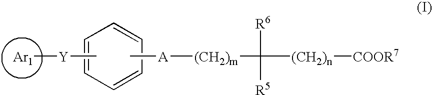
their derivatives, their stereoisomers, their pharmaceutically acceptable salts and their pharmaceutically acceptable compositions.

One aspect of the present invention provides novel compounds of formula (Ia). 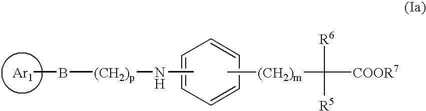
their derivatives, their stereoisomers, their pharmaceutically acceptable salts and their pharmaceutically acceptable compositions.

One aspect of the present invention provides novel compounds of formula (Ib). 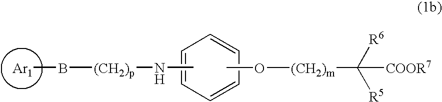
their derivatives, their stereoisomers, their pharmaceutically acceptable salts and their pharmaceutically acceptable compositions.

One aspect of the present invention provides novel compounds of formula (Ib). 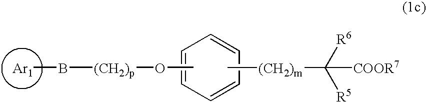
their derivatives, their stereoisomers, their pharmaceutically acceptable salts and their pharmaceutically acceptable compositions.

One aspect of the present invention provides novel compounds of formula (Id) 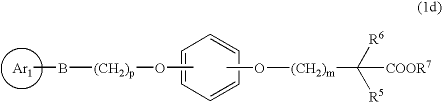
their derivatives, their stereoisomers, their pharmaceutically acceptable salts and their pharmaceutically acceptable compositions.

One aspect of the present invention provides novel compounds of formula (Ie) 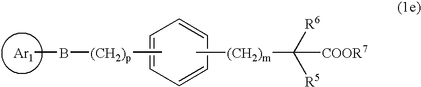
their derivatives, their stereoisomers, their pharmaceutically acceptable salts and their pharmaceutically acceptable compositions.

One aspect of the present invention provides novel compounds of formula (If) 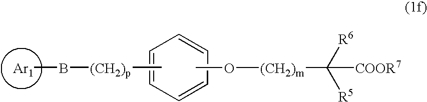
their derivatives, their stereoisomers, their pharmaceutically acceptable salts and their pharmaceutically acceptable compositions.

Further exemplifying the invention is a pharmaceutical composition comprising any of the compounds described above and a pharmaceutically acceptable carrier. Another illustration of the invention is a process for making a pharmaceutical composition comprising combining any of the compounds described above and a pharmaceutically acceptable carrier.

Further illustrating the invention is method for treatment and/or prophylaxis of a condition that requires an agonist of peroxisome proliferator activated receptor in a patient in need thereof, comprising administering to the patient a therapeutically effective amount of any of the compounds described above. Preferably the condition is selected from insulin resistance and dyslipidemia such as diabetes, hypertension, coronary heart disease, atherosclerosis, stroke, peripheral vascular diseases, psoriasis, polycystic ovarian syndrome (PCOS), inflammatory bowel diseases, osteoporosis, myotonic dystrophy, pancreatitis, retinopathy, arteriosclerosis, xanthoma and related disorders.

Another illustration of the invention is method for treatment and/or prophylaxis of the above mentioned diseases using the compounds of the present invention in combination/concomitant with one or more HMG CoA reductase inhibitor; cholesterol absorption inhibitor; antiobesity drug; lipoprotein disorder treatment drug; hypoglycemic agents: insulin; biguanide; sulfonylurea; thiazolidinedione; dual PPARα and γ or a mixture thereof. The compounds of the present, invention in combination with HMG CoA reductase inhibitor, cholesterol absorption inhibitor, antiobesity drug, hypoglycemic agent can be administered together or within such a period to act synergistically.

Further exemplifying the invention is a pharmaceutical composition, containing the compounds the present invention as defined above, their pharmaceutically acceptable salts and one or more HMG CoA reductase inhibitor; cholesterol absorption inhibitor, antiobesity drug; lipoprotein disorder treatment drug; hypoglycemic agents: insulin; biguanide; sulfonylurea; thiazolidinedione; dual PPARα and γ or a mixture thereof in combination with the usual pharmaceutically employed carriers, diluents and the like.

### DETAILED DESCRIPTION OF THE INVENTION

Accordingly the present invention provides compounds of general formula (I), 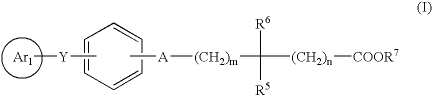
their derivatives, their stereoisomers, their pharmaceutically acceptable salts and their pharmaceutically acceptable compositions.
wherein ring “Ar₁” represents a monocyclic or polycyclic aromatic or partially saturated aromatic polycyclic, which may optionally contain up to 3 heteroatoms selected from N, S or O.
preferably 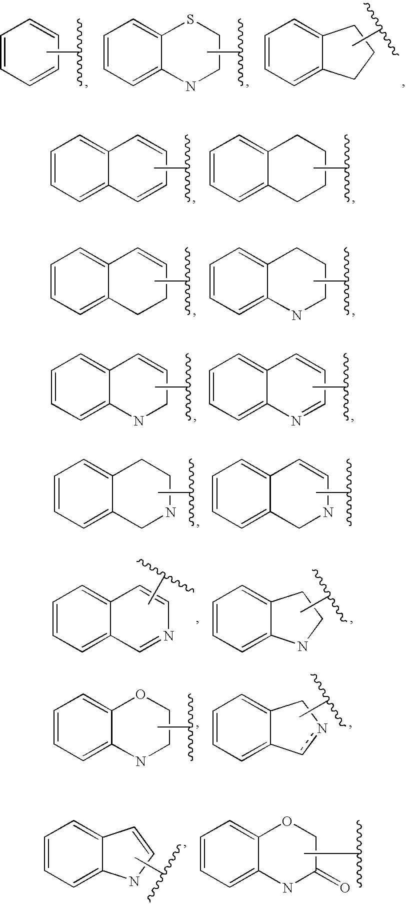
The said monocyclic or polycyclic ring may be unsubstituted or have up to 4 substituents which may be identical or different;
m and n independently represents an integer from 0 to 6;
A represents O, S or a bond;
Y is selected from (CH₂)ₚ, (CH₂)ₚB(CH₂)_{q}, (CH₂)ₚB(CH₂)ₚD(CH₂)_{q}, where p, q and r each independently represents an integer from 0 to 6; B and D independently represents S, O, NR⁴ or a bond, with a proviso that when B and D represents hetero atom p is not zero;

R⁴ represents hydrogen, alkyl, alkenyl, alkynyl, —S(O)₂—R⁸ or —C(O)R⁸ where R⁸ is alkyl, alkoxy;

R⁵ and R⁶ independently represents hydrogen, alkyl, cycloalkyl or alkoxy; R⁵ and R⁶ together may form 3-8 membered cyclic ring which may optionally contains one or two hetero atoms selected from O, S or N;

R⁷ represents hydrogen, optionally substituted groups selected form alkyl, cycloalkyl, alkenyl or alkynyl

The substituent on ring “Ar₁” is selected from halo, nitro, alkyl, hydroxy, hydroxy alkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

R¹ and R² independently represents hydrogen, optionally substituted groups selected from alkyl, alkenyl, alkynyl, cylcoalkyl, heterocyclyl, aryl, heteroaryl.

R³ independently represents hydrogen, optionally substituted groups selected from alkyl, alkenyl, alkynyl, cylcoalkyl, heterocyclyl, aryl, heteroaryl.

Substitutents on R¹, R², R³ and R⁷ are selected from hydrogen, halo, nitro, amino, mono or di substituted amino, hydroxy, alkoxy, carboxy, cyano, alkyl, cycloalkyl, alkoxy, haloalkoxy, haloalkyl, cycloalkyl, aryl, heterocyclyl, heteroaryl.

One embodiment of the present invention is a compound of formula (I) as described by formula (Ia) 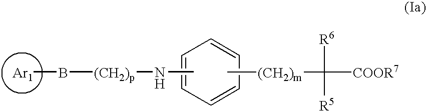
their derivatives, their stereoisomers, their pharmaceutically acceptable salts and their pharmaceutically acceptable compositions.
wherein “Ar₁” represents optionally substituted group selected from 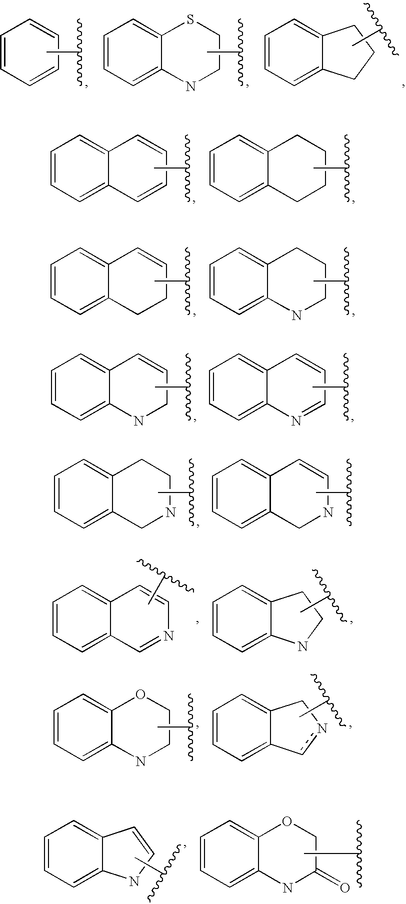
p and m independently represents an integer from 0 to 6;
B represents S, O or NR⁴ or a bond;

The substituent on ring “Ar₁” is selected from halo, nitro, alkyl, hydroxy, hydroxy alkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

And all other symbols are as defined above.

Representative compounds in accordance with the present invention are presented in Table 1. This table is not intended to be exclusive of the compounds of the present invention, but rather exemplary of the compounds of formula (Ia), that are encompassed by this invention.

TABLE 1

| S. No. | Structure | IUPAC Name |
|---|---|---|
| 1. | 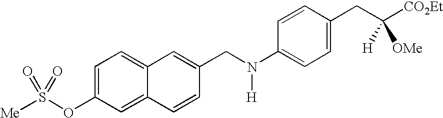 | (S)-Ethyl 2-methoxy-3-[4-{6- methanesulfonyloxynapth-2- ylmethylamino}phenyl]propanoate |
| 2. | 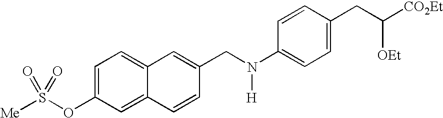 | Ethyl 2-ethoxy-3-[4-{6- methanesulfonyloxynapth-2- ylmethylamino}phenyl]propanoate |
| 3. | 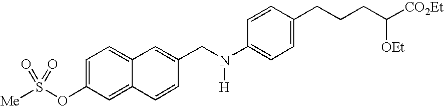 | Ethyl 2-ethoxy-5-[4-{6- methanesulfonyloxynapth-2- ylmethylamino}phenyl]pentanoate |
| 4. | 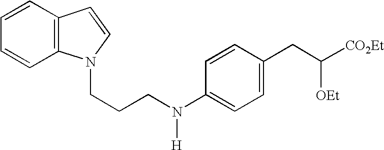 | Ethyl 2-ethoxy-3-[4-{3-(indol-1-yl) propyl amino}phenyl]propanoate |
| 5. | 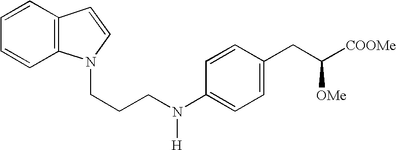 | (S)-Methyl 2-methoxy-3-[4-{3- (indol-1-yl)propylamino}phenyl]propanoate |
| 6. | 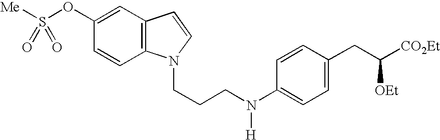 | (S)-Ethyl-2-ethoxy-3-[4-{3-(5- methanesulfonyloxyindol-1-yl) propylamino}phenyl]propanoate |
| 7. | 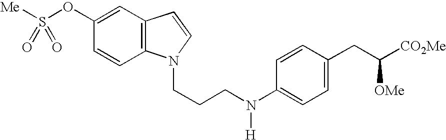 | S)-Methyl-2-methoxy-3-[4-{3-(5- methanesulfonyloxyindol-1-yl) propylamino}phenyl]propanoate |
| 8. | 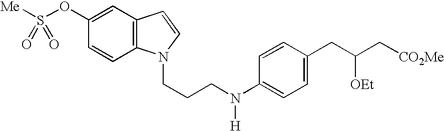 | (S)-Methyl 3-ethoxy-4-[4-{3-(5- methanesulfonyloxyindol-1-yl) propylamino}phenyl]butanoate |
| 9. | 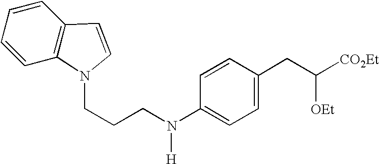 | Ethyl 2-ethoxy-3-[4-{3-(2,3- dihydroindol-1-yl)propylamino}phenyl]propanoate |
| 10. | 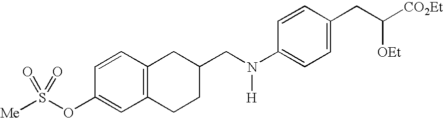 | Ethyl 2-ethoxy-3-[4-{(6- methanesulfonyloxy-1,2,3,4- tetrahydronapth-2-yl)methylamino}phenyl]propanoate |
| 11. | 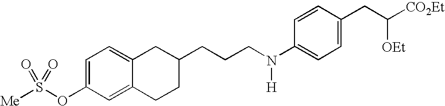 | Ethyl 2-ethoxy-3-[4-{3-(6-methane sulfonyloxy-1,2,3,4- tetrahydronapth-2-yl)propylamino}phenyl]propanoate |
| 12. | 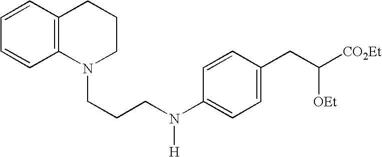 | Ethyl 2-ethoxy-3-[4-{3-(1,2,3,4- tetrahydroquinolyn-1-yl) propylamino}phenyl]propanoate |
| 13. | 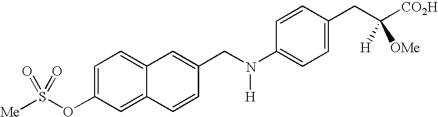 | (S)-2-methoxy-3-[4-(6- methanesulfonyloxynapth-2- ylmethylamino)phenyl]propanoic acid |
| 14. | 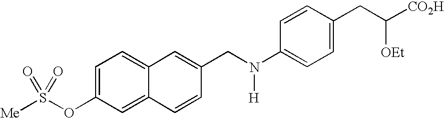 | 2-ethoxy-3-[4-{6- methanesulfonyloxynapth-2- ylmethylamino}phenyl]propanoic acid |
| 15. | 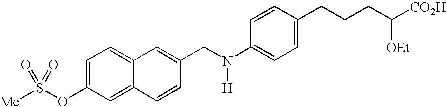 | 2-Ethoxy-5-[4-{6- methanesulfonyloxynapth-2- ylmethylamino}phenyl]pentatonic acid |
| 16. | 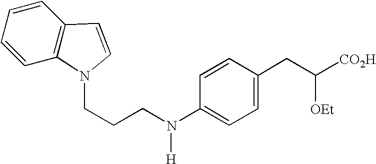 | 2-ethoxy-3-[4-{3-(indol-1-yl) propyl amino}phenyl]propanoic acid |
| 17. | 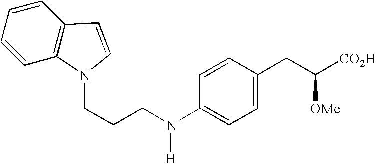 | (S)-2-methoxy-3-[4-{3-(indol-1-yl) propyl amino}phenyl]propanoic acid |
| 18. | 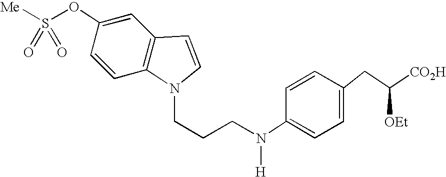 | (S)-2-ethoxy-3-[4-{3-(5- methanesulfonyloxyindol-1-yl) propylamino}phenyl]propanoic acid |
| 19. | 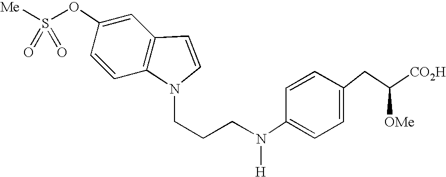 | S)-2-methoxy-3-[4-{3-(5- methanesulfonyloxyindol-1-yl) propylamino}phenyl]propanoic acid |
| 20. | 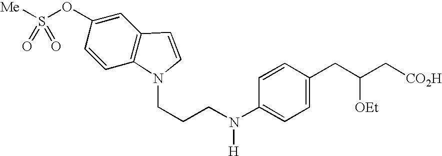 | S)-3-ethoxy-4-[4-{3-(5- methanesulfonyloxyindol-1-yl) propylamino}phenyl]butanoic acid |
| 21. | 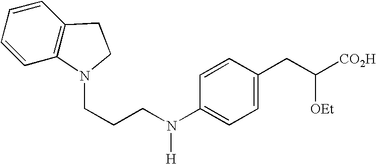 | 2-ethoxy-3-[4-{3-(2,3- dihydroindol-1-yl)propylamino}phenyl]propanoic acid |
| 22. | 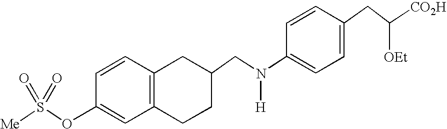 | 2-ethoxy-3-[4-{(6- methanesulfonyloxy-1,2,3,4- tetrahydronapth-2-yl)methylamino}phenyl]propanoic acid |
| 23. | 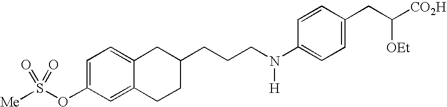 | 2-ethoxy-3-[4-{3-(6- methanesulfonyloxy-1,2,3,4- tetrahydronapth-2-yl)propylamino}phenyl]propanoic aci |
| 24. | 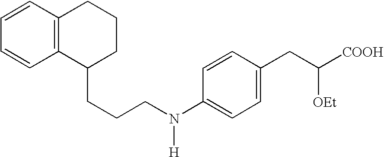 | 2-ethoxy-3-[4-{3-(1,2,3,4- tetrahydroquinolyn-1-yl) propylamino}phenyl]propanoic acid |
| 25. | 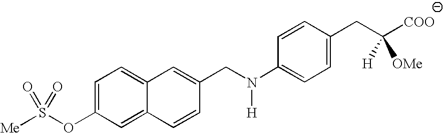 | (S)-2-methoxy-3-[4-{6- methanesulfonyloxynapth-2- ylmethylamino}phenyl]propanoic acid Arginine salt |
| | 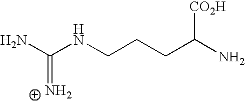 | |
| 26. | 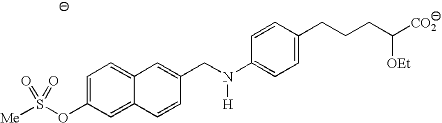 | 2-Ethoxy-5-[4- (6-methanesulfonyl oxynapth-2-ylmethylamino}phenyl]pentatonic acid Arginine salt |
| | 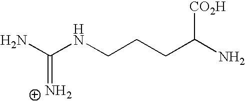 | |
| 27. | 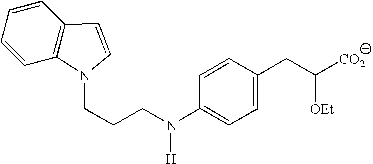 | 2-ethoxy-3-[4-{3-(indol-1-yl) propyl amino}phenyl]propanoic acid Arginine salt |
| | 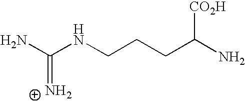 | |
| 28. | 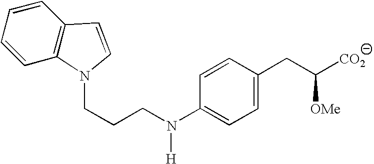 | (S)-2-methoxy-3-[4-{3-(indol-1-yl) propyl amino)phenyl]propanoic acid Arginine salt |
| | 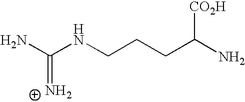 | |
| 29. | 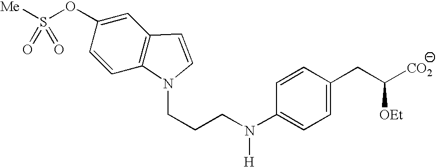 | (S)-2-ethoxy-3-[4-{3-(5- methanesulfonyl oxyindol-1-yl) propylamino}phenyl]propanoic acid Arginine salt |
| | 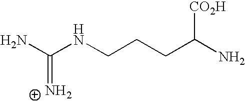 | |
| 30. | 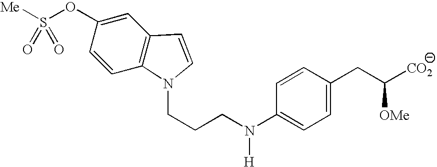 | (S)-2-methoxy-3-[4-{3-(5- methanesulfonyl oxyindol-1-yl) propylamino)phenyl]propanoic acid Arginine salt |
| | 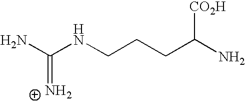 | |
| 31. | 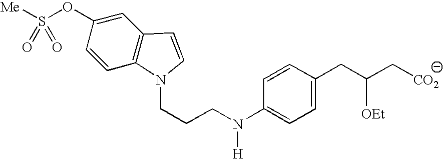 | (S)-3-ethoxy-4-[4-{3-(5- methanesulfonyloxyindol-1-yl) propylamino)phenyl]butanoic acid Arginine salt |
| | 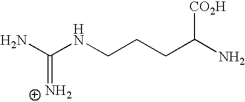 | |
| 32. | 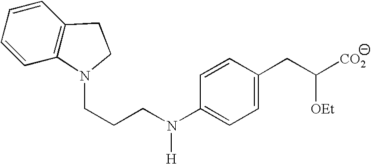 | 2-ethoxy-3-[4-{3-(2,3- dihydroindol-1-yl)propylamino) phenyl]propanoic acid Arginine salt |
| | 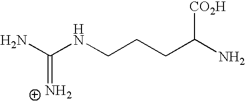 | |
| 33. | 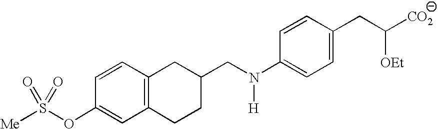 | 2-ethoxy-3-[4-{(6- methanesulfonyloxy-1,2,3,4- tetrahydronapth-2-yl)methylamino}phenyl]propanoic acid Arginine salt |
| | 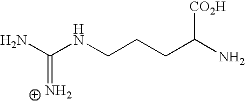 | |
| 34. | 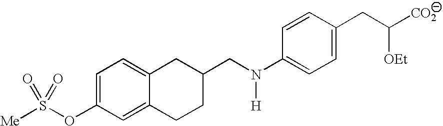 | 2-ethoxy-3-[4-{3-(6- tetrahydronapth-2-yl)propylamino}phenyl]propanoic acid Arginine salt |
| | 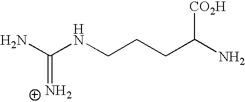 | |
| 35. | 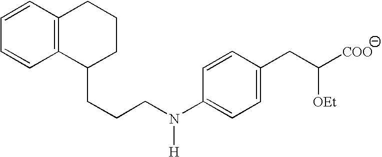 | 2-ethoxy-3-[4-{3-(1,2,3,4- tetrahydroquinolyn-1-yl) propylamino}phenyl]propanoic acid Arginine salt |
| | 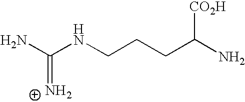 | |

Another embodiment of the present invention is a compound of formula (Ia) where “Ar₁” is substituted with —OSO₂R³, wherein R³ is as defined above preferably optionally substituted groups selected from alkyl or aryl.

And all other symbols are as defined above.

Another embodiment of the present invention is a compound of formula (I) as described by formula (Ib) 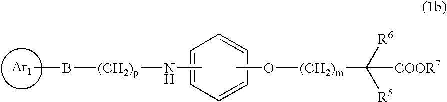
their derivatives, their stereoisomers, their pharmaceutically acceptable salts and their pharmaceutically acceptable compositions.
wherein “Ar₁” represents optionally substituted group selected from 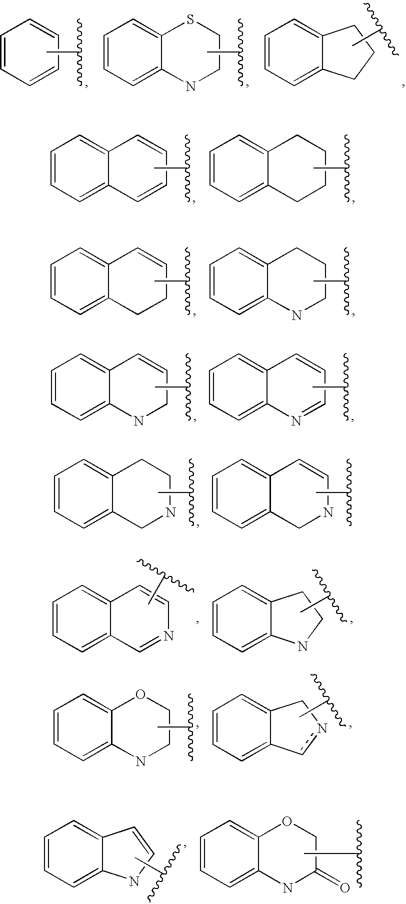
p and m independently represents an integer from 0 to 6;
B represents S, O or NR⁴ or a bond;

The substituent on ring “Ar₁” is selected from halo, nitro, alkyl, hydroxy, hydroxyalkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

And all other symbols are as defined above.

Representative compounds in accordance with the present invention are presented in Table 2. This table is not intended to be exclusive of the compounds of the present invention, but rather exemplary of the compounds of formula (Ib), that are encompassed by this invention.

TABLE 2

| S. No. | Structure | IUPAC Name |
|---|---|---|
| 1. | 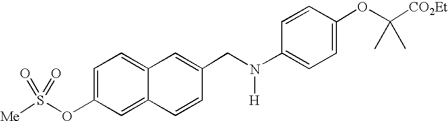 | Ethyl 2-methyl-2-[4-{6- methanesulfonyloxynapth-2- ylmethylamino}phenoxy]propanoate |
| 2. | 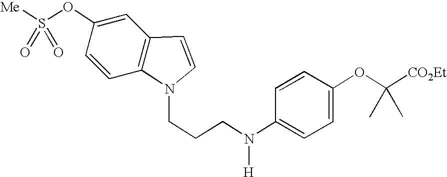 | Ethyl 2-methyl-2-[4-{3-(5- methanesulfonyloxyindol-1-yl) propylamino}phenoxy]propanoate |
| 3. | 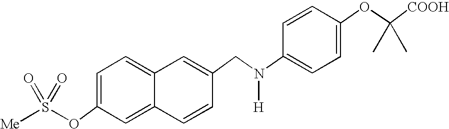 | 2-methyl-2-[4-{6- methanesulfonyloxynapth-2- ylmethylamino}phenoxy]propanoic acid |
| 4. | 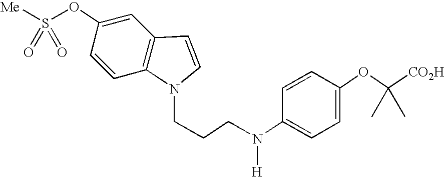 | 2-methyl-2-[4-{3-(5- methanesulfonyloxyindol-1-yl) propylamino}phenoxy]propanoic acid |

Another embodiment of the present invention is a compound of formula (Ib) where “Ar₁” is substituted with —OSO₂R³, wherein R³ is as defined above preferably optionally substituted groups selected from alkyl or aryl.

And all other symbols are as defined above.

Another embodiment of the present invention is a compound of formula (I) as described by formula (Ic) 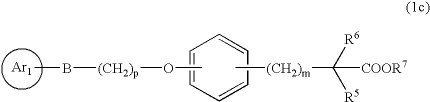
their derivatives, their stereoisomers, their pharmaceutically acceptable salts and their pharmaceutically acceptable compositions.
wherein “Ar₁” represents optionally substituted group selected from 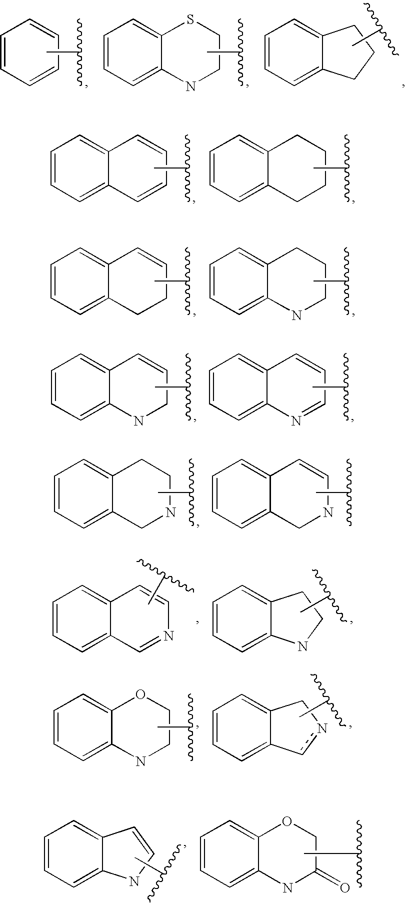
p and m independently represents an integer from 0 to 6;
B represents S, O or NR⁴ or a bond;

The substituent on ring “Ar₁” is selected from halo, nitro, alkyl, hydroxy, hydroxyalkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

And all other symbols are as defined above.

Another embodiment of the present invention is a compound of formula (Ic) where “Ar₁” is substituted with —OSO₂R³, wherein R³ is as defined above preferably optionally substituted groups selected from alkyl or aryl.

And all other symbols are as defined above.

Another embodiment of the present invention is a compound of formula (I) as described by formula (Id) 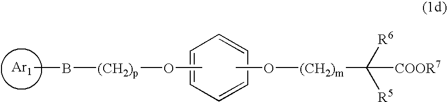
their derivatives, their stereoisomers, their pharmaceutically acceptable salts and their pharmaceutically acceptable compositions.
wherein “Ar₁” represents optionally substituted group selected from 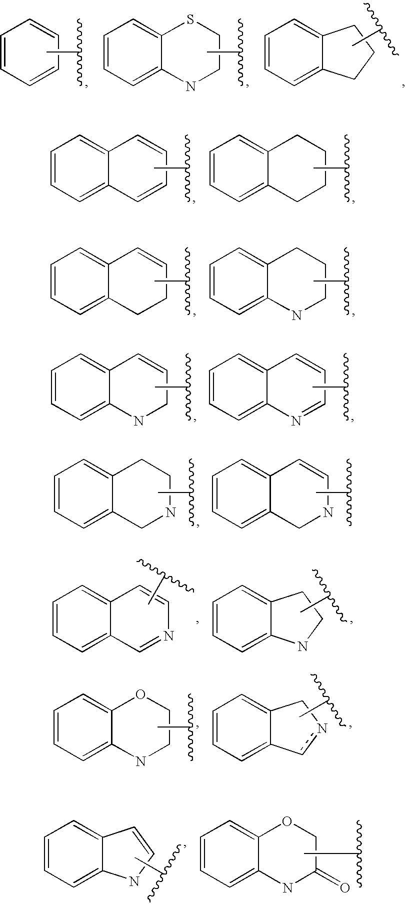
p and m independently represents an integer from 0 to 6;
B represents S, O or NR⁴ or a bond;

The substituent on ring “Ar₁” is selected from halo, nitro, alkyl, hydroxy, hydroxyalkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

And all other symbols are as defined above.

Representative compounds in accordance with the present invention are presented in Table 3. This table is not intended to be exclusive of the compounds of the present invention, but rather exemplary of the compounds of formula (Id), that are encompassed by this invention.

TABLE 3

| | | |
|---|---|---|
| S. No. | Structure | IUPAC Name |

| | | |
|---|---|---|
| 1. | 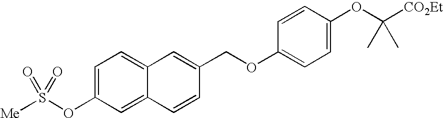 | Ethyl 2-methyl-2-[4-{6- methanesulfonyloxynapth-2- ylmethoxy}phenoxy]propanoate |
| 2. | 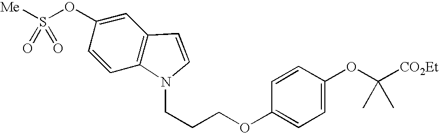 | Ethyl 2-methyl-2-[4-{3-(5- methanesulfonyloxyindol-1-yl) propyloxy}phenoxy]propanoate |
| 3. | 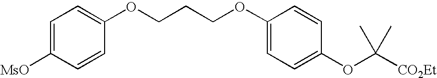 | Ethyl 2-methyl-2-[4-{3-(4- methanesulfonyloxyphenoxy) propyloxy}phenoxy]propanoate |
| 4. | 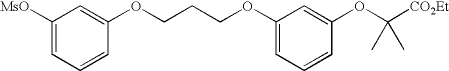 | Ethyl 2-methyl-2-[3-{3-(3- methanesulfonyloxyphenoxy) propyloxy}phenoxy]propanoate |
| 5. | 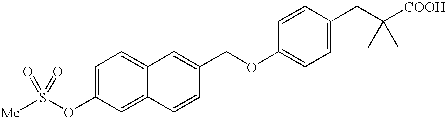 | 2-methyl-2-[4-{6- methanesulfonyloxynapth-2- ylmethoxy}phenoxy]propanoic acid |
| 6. | 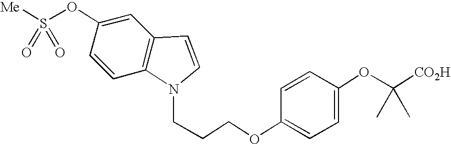 | 2-methyl-2-[4-{3-(5- methanesulfonyloxyindol-1-yl) propyloxy}phenoxy]propanoic acid |
| 7. | 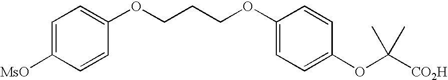 | 2-Methyl-2-[4-{3-(4 methanesulfonyloxyphenoxy) propyloxy}phenoxy]propanoic acid |
| 8. | 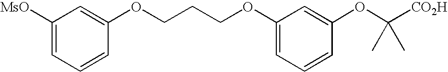 | 2-Methyl-2-[3-{3-(3- methanesulfonyloxyphenoxy) propyloxy}phenoxy]propanoic acid |
| 9. | 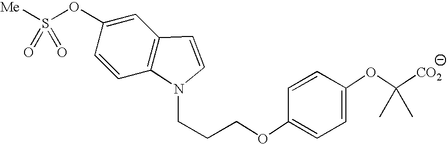 | 2-methyl-2-[4-{3-(5- methanesulfonyloxyindol-1-yl) propyloxy}phenoxy]propanoic acid Arginine salt |
| | 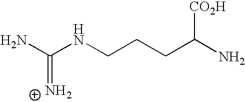 | |
| 10. | 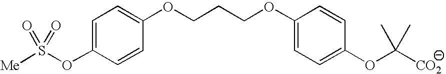 | 2-Methyl-2-[4-55 3-(4- methanesulfonyloxyphenoxy) propyloxy}phenoxy]propanoic acid Arginine salt |
| | 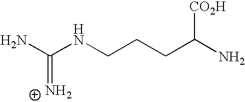 | |
| 11. | 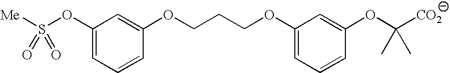 | 2-Methyl-2-[3-{3-(3- methanesulfonyloxyphenoxy) propyloxy}phenoxy]propanoic acid Arginine salt |
| | 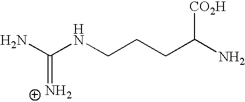 | |
| 12. | 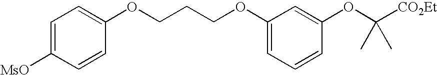 | Ethyl 2-methyl-2-[3-{3-(4- methanesulfonyloxyphenoxy) propyloxy}phenoxy]propanoate |
| 13. | 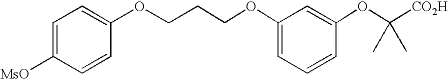 | 2-Methyl-2-[3-{3-(4- methanesulfonyloxyphenoxy) propyloxy}phenoxy]propanoic acid |
| 14. | 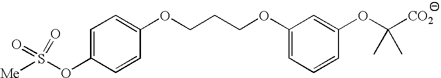 | 2-Methyl-2-[3-{3-(4- methanesulfonyloxyphenoxy) propyloxy}phenoxy]propanoic acid Arginine salt |
| | 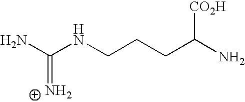 | |
| 15. | 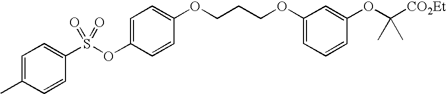 | Ethyl 2-methyl-2-[3-{3-(4-para- toluenesulfonyloxy)phenoxy) propyloxy}phenoxy]propanoate |
| 16. | 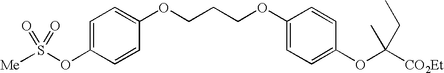 | Ethyl 2-methyl-2-[4-{3-(4- methanesulfonyloxyphenoxy) propyloxy}phenoxy]butanoate |
| 17. | 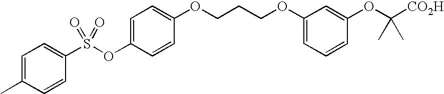 | 2-methyl-2-[3-{3-(4-(para- toluenesulfonyloxy)phenoxy) propyloxy}phenoxy]propanoic acid |
| 18. | 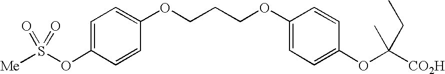 | 2-Methyl-2-[4-{3-(4- methanesulfonyloxyphenoxy) propyloxy}phenoxy]butanoic acid |
| 19. | 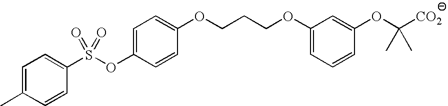 | 2-Methyl-2-[3-{3-(4-para- toluenesulfonyloxy)phenoxy) propyloxy}phenoxy]propanoic acid, arginine salt |
| | 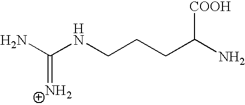 | |
| 20. | 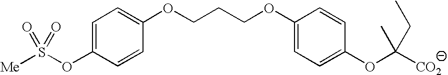 | 2-Methyl-2-[4-{3-(4- methanesulfonyloxyphenoxy) propyloxy}phenoxy]butanoic acid, arginine salt |
| | 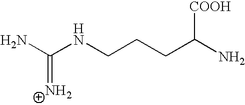 | |

Another embodiment of the present invention is a compound of formula (Id) where “Ar₁” is substituted with —OSO₂R³, wherein R³ is as defined above preferably optionally substituted groups selected from alkyl or aryl.

Another embodiment of the present invention is a compound of formula (I) as described by formula (Ie) 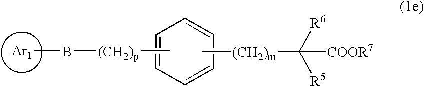
their derivatives, their stereoisomers, their pharmaceutically acceptable salts and their pharmaceutically acceptable compositions.
wherein “Ar₁” represents optionally substituted group selected from 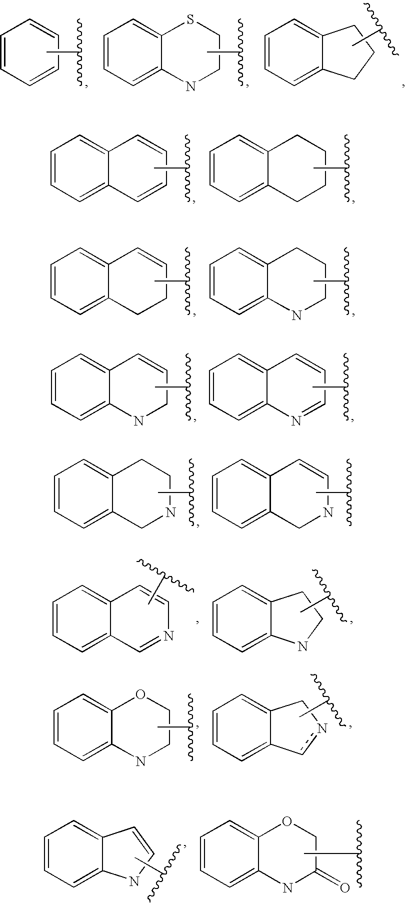
p and m independently represents an integer from 0 to 6;
B represents S, O or NR⁴ or a bond;

The substituent on ring “Ar₁” is selected from halo, nitro, alkyl, hydroxy, hydroxyalkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

And all other symbols are as defined above.

Another embodiment of the present invention is a compound of formula (Ie) where “Ar₁” is substituted with —OSO₂R³, wherein R³ is as defined above preferably optionally substituted groups selected from alkyl or aryl.

And all other symbols are as defined above.

Another embodiment of the present invention is a compound of formula (I) as described by formula (If) 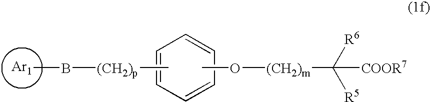
their derivatives, their stereoisomers, their pharmaceutically acceptable salts and their pharmaceutically acceptable compositions.
wherein “Ar₁” represents optionally substituted group selected from 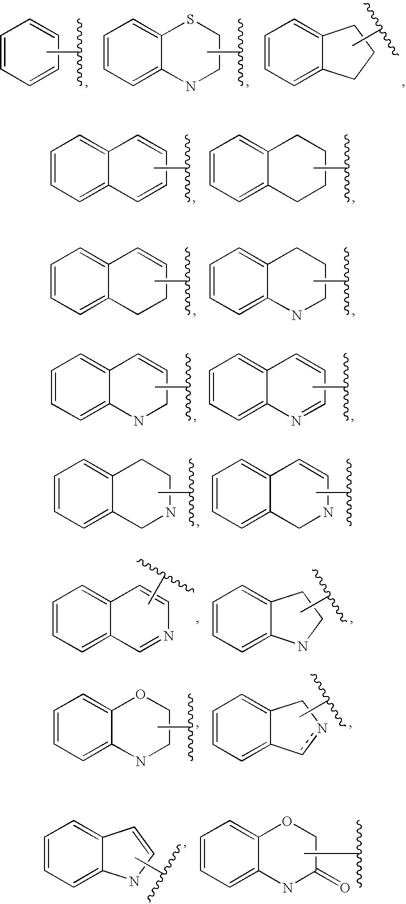
p and m independently represents an integer from 0 to 6;
B represents S, O or NR⁴ or a bond;

The substituent on ring “Ar₁” is selected from halo, nitro, alkyl, hydroxy, hydroxyalkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³.

And all other symbols are as defined above.

Representative compounds in accordance with the present invention are presented in Table 4. This table is not intended to be exclusive of the compounds of the present invention, but rather exemplary of the compounds of formula (If), that are encompassed by this invention.

TABLE 4

| | | |
|---|---|---|
| S. No. | Structure | IUPAC Name |

| | | |
|---|---|---|
| 1. | 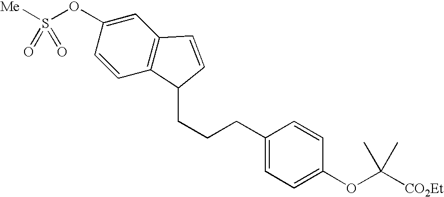 | Ethyl 2-methyl-2-[4-{3-(5- methanesulfonyloxyindol-1-yl) propyl}phenoxy]propanoate |
| 2. | 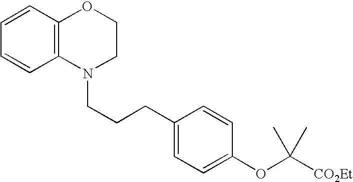 | Ethyl 2-methyl-2-[4-{3-(3,4- dihydro-2H-bezo[b][1,4]Oxazin- 4-yl)propyl}phenoxy]propanoate |
| 3. | 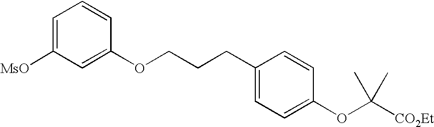 | Ethyl 2-methyl-2-[4-{3-(3- methanesulfonyloxyphenoxy) propyl}phenoxy]propanoate |
| 4. | 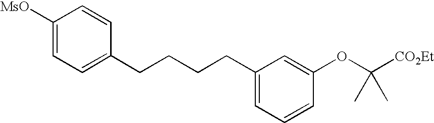 | Ethyl 2-methyl-2-[3-{3-(4- methanesulfonyloxyphenoxy) propyl}phenoxy]propanoate |
| 5. | 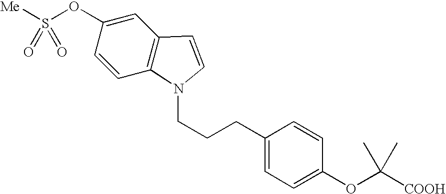 | 2-methyl-2-[4-{3-(5- methanesulfonyloxyindol-1-yl) propyl}phenoxy]propanoic acid |
| 6. | 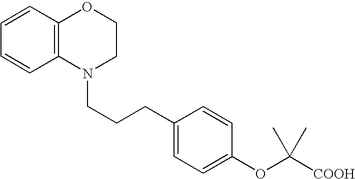 | 2-methyl-2-[4-{3-(3,4-dihydro- 2H-bezo[b][1,4]Oxazin-4-yl) propyl}phenoxy]propanoic acid |
| 7. | 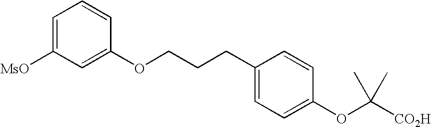 | 2-Methyl-2-[4-{3-(3- methanesulfonyloxyphenoxy) propyl}phenoxy]propanoic acid |
| 8. | 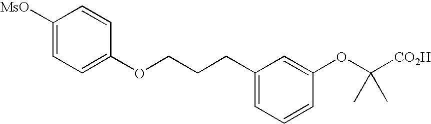 | 2-Methyl-2-[3-{3-(4- methanesulfonyloxyphenoxy) propyl}phenoxy]propanoic acid |
| 9. | 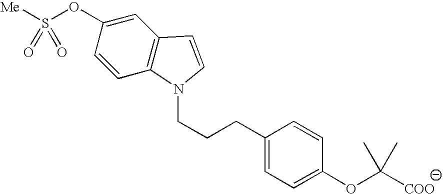 | 2-methyl-2-[4-{3-(5- methanesulfonyloxyindol-1-yl) propyl}phenoxy]propanoic acid Arginine salt |
| | 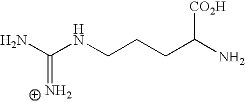 | |
| 10. | 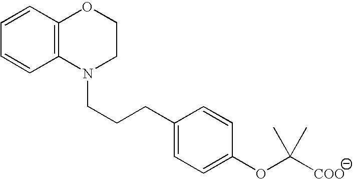 | 2-methyl-2-[4-{3-(3,4-dihydro-2H- bezo[b][1,4]Oxazin-4-yl)propyl}phenoxy]propanoic acid Arginine salt |
| | 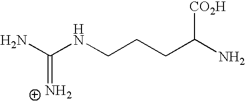 | |
| 11. | 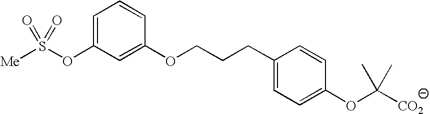 | 2-Methyl-2-[4-{3-(3- methanesulfonyloxyphenoxy) propyl}phenoxy]propanoic acid Arginine salt |
| | 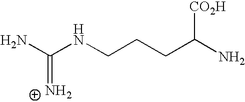 | |
| 12. | 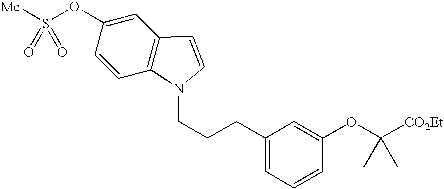 | Ethyl 2-methyl-2-[3-{3-(5- methanesulfonyloxyindol-1-yl) propyl}phenoxy]propanoate |
| 13. | 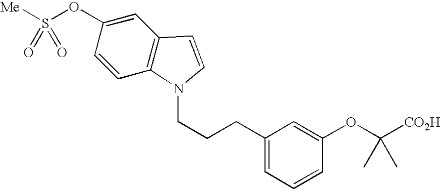 | 2-methyl-2-[3-{3-(5- methanesulfonyloxyindol-1-yl) propyl}phenoxy]propanoic acid |
| 14. | 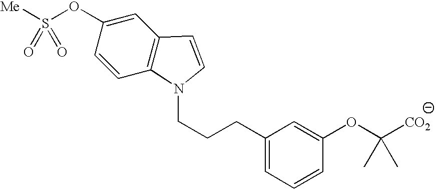 | 2-methyl-2-[3-{3-(5- methanesulfonyloxyindol-1-yl) propyl}phenoxy]propanoic acid Arginine salt |
| | 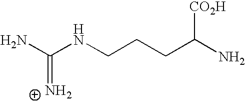 | |
| 15. | 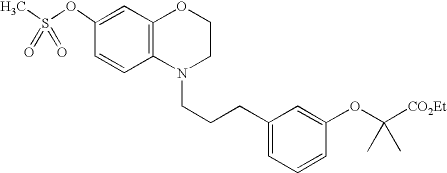 | Ethyl-2-methyl-2-[3-{3-(7- Methanesulfonyloxy-3,4-dihydro- 2H-bezo[b][1,4]oxazin-4-yl) propyl}phenoxy]propanoate. |
| 16. | 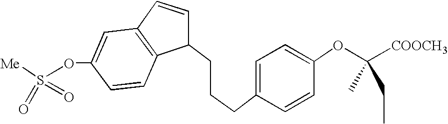 | (+) Methyl (R)-2-methyl-2-[4-{3- (5-methanesulfonyloxyindol-1- yl)propyl}phenoxy]butanoate |
| 17. | 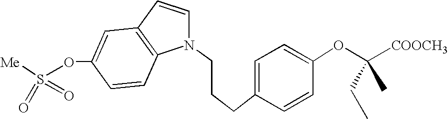 | (−) Methyl (S)-2-methyl-2-[4-{3-(5- methanesulfonyloxyindol-1- yl)propyl}phenoxy]butanoate |
| 18. | 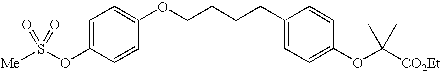 | Ethyl 2-methyl-2-[4-{4-(4- methanesulfonyloxyphenoxy)butyl}phenoxy]propanoate |
| 19. | 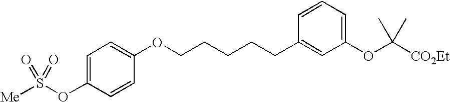 | Ethyl 2-methyl-2-[3-{5-(4- methanesulfonyloxyphenoxy)pentyl}phenoxy]propanoate |
| 20. | 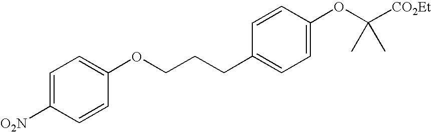 | Ethyl 2-methyl-2-[3-{5-(4- nitrophenoxy)propyl}phenoxy]propanoate |
| 21. | 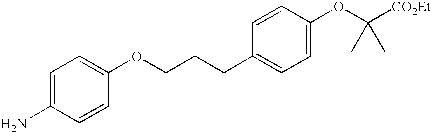 | Ethyl 2-methyl-2-[3-{5-(4- aminophenoxy)propyl}phenoxy]propanoate |
| 22. | 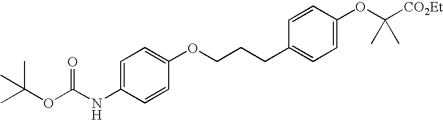 | Ethyl 2-methyl-2-[4-{3-(4-(tert- butyloxycaxbonylamino)phenoxy) propyl}phenoxy]propanoate |
| 23. | 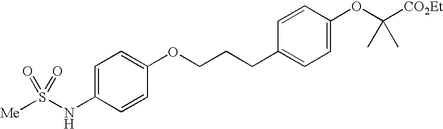 | Ethyl 2-methyl-2-[4-{3-(4- (methanesulfonylamino)phenoxy) propyl}phenoxy]propanoate |
| 24. | 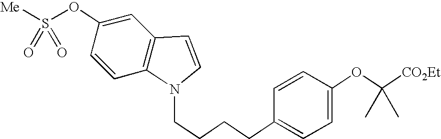 | Ethyl 2-methyl-2-[4-{4-(5- methanesulfonyloxyindol-1- yl)butyl}phenoxy]propanoate |
| 25. | 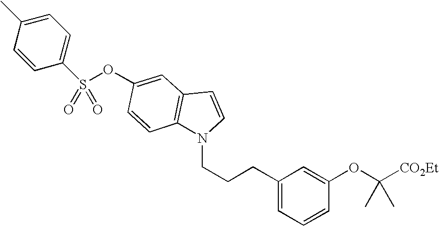 | Ethyl 2-methyl-2-[3-{3-(5-(para- toluenesulfonyloxy)indol-1- yl)propyl}phenoxy]propanoate |
| 26. | 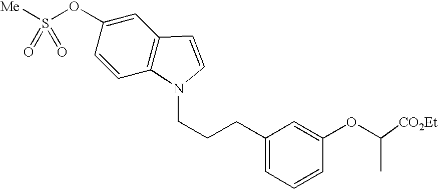 | Ethyl 2-[3-{3-(5- methanesulfonyloxyindol-1-yl) propyl}phenoxy]propanoate |
| 27. | 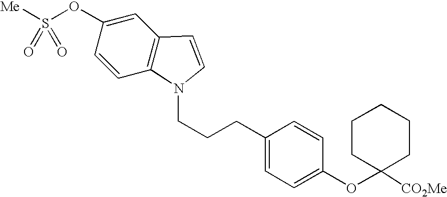 | 1-[4-{3-(5- Methanesulfonyloxyindol-1- yl)propyl}phenoxy]cyclohexane-1- carboxylic acid, methyl ester |
| 28. | 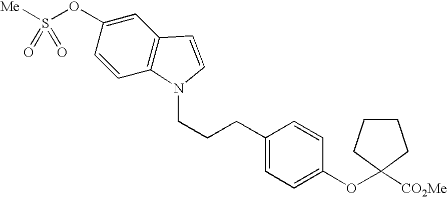 | 1-[4-{3-(5- methanesulfonyloxyindol-1- yl)propyl}phenoxy]cyclopentane-1- carboxylic acid, methyl ester |
| 29. | 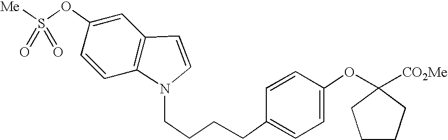 | 1-[4-{3-(5- methanesulfonyloxyindol-1- yl)butyl}phenoxy]cyclopentane-1- carboxylic acid, methyl ester |
| 30. | 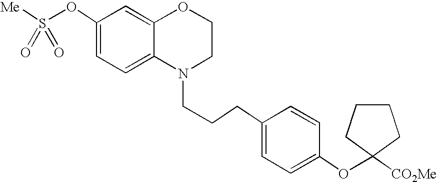 | 1-[4-{3-(7-Methanesulfonyloxy-3, 4-dihydro-2H-bezo[b][1,4]oxazin-4- yl)propyl}phenoxy]cyclopentane-1- carboxylic acid, methyl ester |
| 31. | 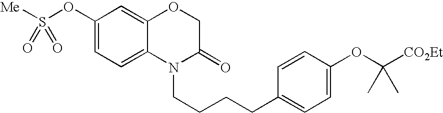 | Ethyl 2-methyl-2-[4-{4-(7- methanesulfonyloxy-3,4-dihydro- 2H-bezo[b][1,4]oxazin-3-on-4- yl)butyl}phenoxy]propanoate |
| 32. | 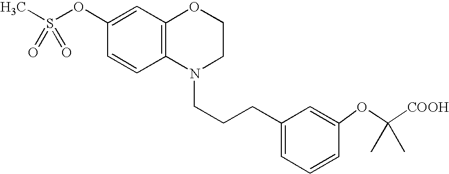 | 2-Methyl-2-[3-{3-(7- Methanesulfonyloxy-3,4-dihydro- 2H-bezo[b][1,4]oxazin-4-yl) propyl}phenoxy]propanoic acid |
| 33. | 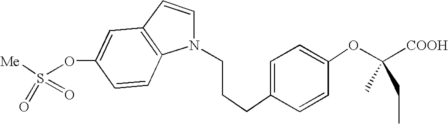 | (R)-(+)-2-methyl-2-[4-{3-(5- methanesulfonyloxyindol-1-yl) propyl}phenoxy]butanoic acid |
| 34. | 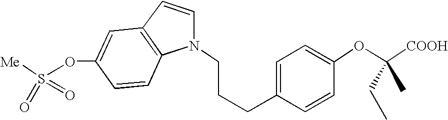 | (S)-(-31 )-2-methyl-2-[4-{3-(5-nl methanesulfonyloxyindol-1- yl)propyl}phenoxy]butanoic acid |
| 35. | 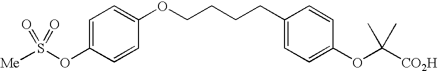 | 2-Methyl-2-[4-{4-(4- methanesulfonyloxyphenoxy) butyl}phenoxy]propanoic acid |
| 36. | 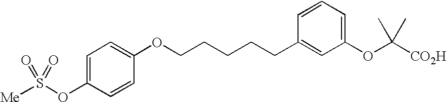 | 2-Methyl-2-[3-{5-(4- methanesulfonyloxyphenoxy)pentyl}phenoxy]propanoic acid |
| 37. | 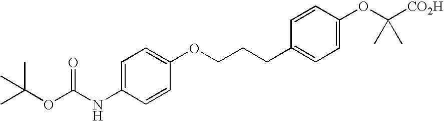 | 2-Methyl-2-[4-{3-(4-tert- butyloxycarbonylamino)phenoxy) propyl}phenoxy]propanoic acid |
| 38. | 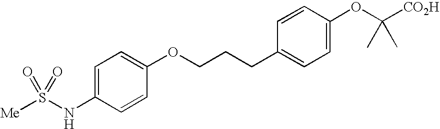 | 2-Methyl-2-[4-{3-(4- (methanesulfonylamino)phenoxy) propyl}phenoxy]propanoic acid |
| 39. | 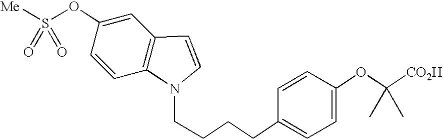 | 2-Methyl-2-[4-{4-(5- methanesulfonyloxyindol- 1yl)butyl}phenoxy]propanoic acid |
| 40. | 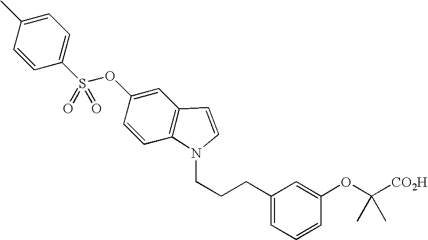 | 2-Methyl-2-[3-{3-(5-(para- toluenesulfonyloxy)indol-1- yl)propyl}phenoxy]propanoic acid |
| 41. | 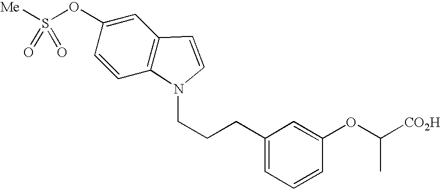 | 2-[3-{3-(5- Methanesulfonyloxyindol-1- yl)propyl}phenoxy]propanoic acid |
| 42. | 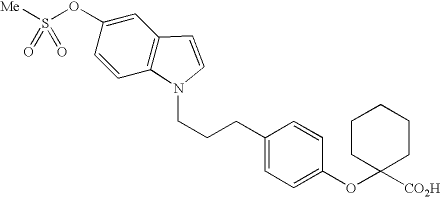 | 1-[4-{3-(5- methanesulfonyloxyindol-1- yl)propyl}phenoxy]cyclohexane-1- carboxylic acid |
| 43. | 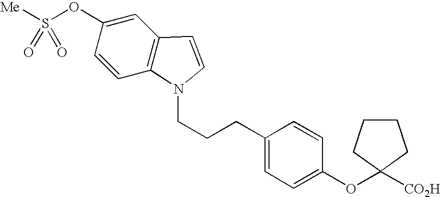 | 1-[4-{3-(5- Methanesulfonyloxyindol-1- yl)propyl}phenoxy]cyclopentane-1- carboxylic acid |
| 44. | 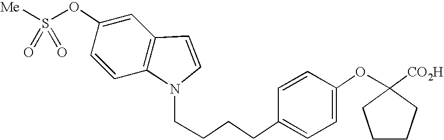 | 1-[4-{3-(5- methanesulfonyloxyindol-1- yl)butyl}phenoxy]cyclopentane-1- carboxylic acid |
| 45. | 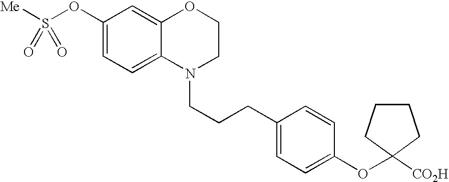 | 1-[4-{3-(7-Methanesulfonyloxy-3, 4-dihydro-2H-bezo[b][1,4]oxazin-4-yl)propyl}phenoxy]cyclopentane-1-carboxylic acid |
| 46. | 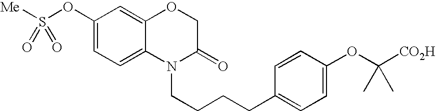 | 2-Methyl-2-[4-{4-(7- methanesulfonyloxy-3,4-dihydro- 2H-bezo[b][1,4]oxazin-3-on-4- yl)butyl}phenoxy]propanoic acid |
| 47. | 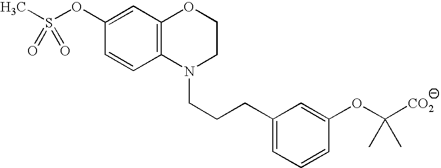 | 2-Methyl-2-[3-{3-(7- Methanesulfonyloxy-3,4-dihydro- 2H-bezo[b][1,4]oxazin-4-yl) propyl}phenoxy]propanoic acid, Arginine salt |
| | 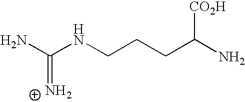 | |
| 48. | 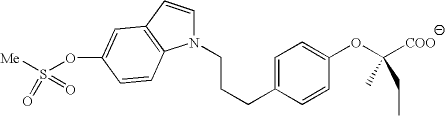 | (R)-(+)-2-methyl-2-[4-{3-(5- methanesulfonyloxyindol-1-yl) propyl}phenoxy]butanoic acid, Arginine salt |
| | 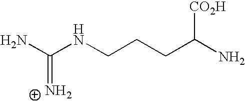 | |
| 49. | 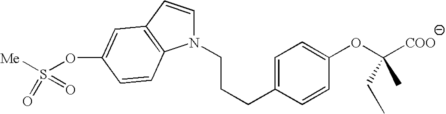 | (S)-(−)-2-methyl-2-[4-{3-(5- methanesulfonyloxyindol-1-yl) propyl}phenoxy]butanoic acid, Arginine salt |
| | 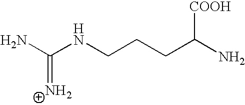 | |
| 50. | 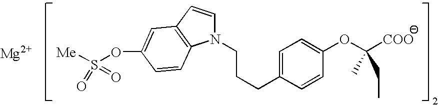 | (racemic) Methyl-2-methyl-2-[4- {3-(5-methanesulfonyloxyindol-1- yl)propyl}phenoxy]butanoic acid Magnesium salt |
| 51. | 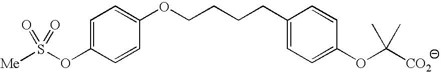 | 2-Methyl-2-[4-{4-(4- methanesulfonyloxyphenoxy)butyl}phenoxy]propanoic acid, arginine salt |
| | 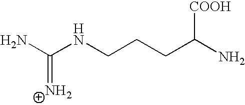 | |
| 52. | 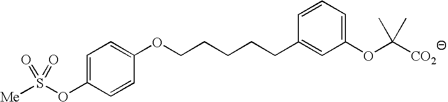 | 2-Methyl-2-[3-{5-(4- methanesulfonyloxyphenoxy)pentyl}phenoxy]propanoic acid, arginine salt |
| | 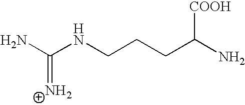 | |
| 53. | 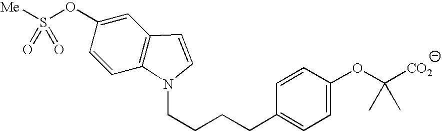 | 2-Methyl-2-[4-{4-(5-methane sulfonyloxyindol-1yl)butyl}phenoxy]propanoic acid, arginine salt |
| | 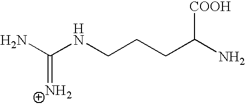 | |
| 54. | 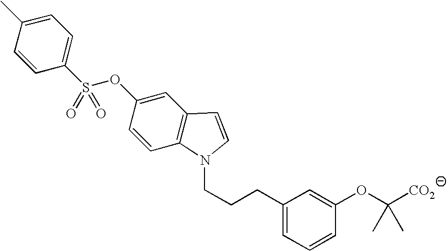 | 2-Methyl-2-[3-{3-(5-(para- toluenesulfonyloxy)indol-1- yl)propyl}phenoxy]propanoic acid, arginine salt |
| | 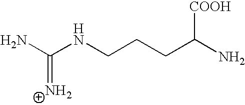 | |
| 55. | 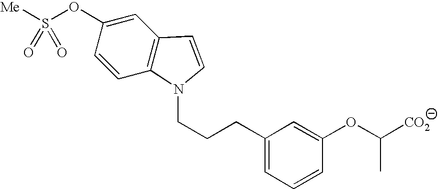 | 2-[3-{3-(5- Methanesulfonyloxyindol-1- yl)propyl}phenoxy]propanoic acid, arginine |
| | 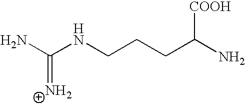 | |
| 56. | 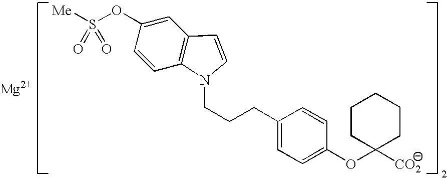 | 1-[4-{3-(5- methanesulfonyloxyindol-1- yl)propyl}phenoxy]cyclohexane-1- carboxylic acid, magnesium salt |
| 57. | 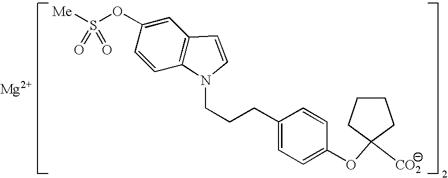 | 1-[4-{3-(5- Methanesulfonyloxyindol-1- yl)propyl}phenoxy]cyclopentane-1- carboxylic acid, magnesium salt |
| 58. | 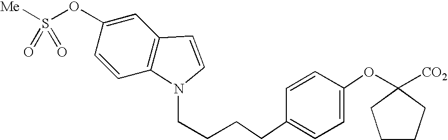 | 1-[4-{3-(5- methanesulfonyloxyindol-1- yl)butyl}phenoxy]cyclopentane-1- carboxylic acid, arginine salt |
| | 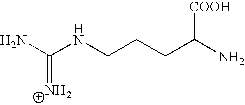 | |
| 59. | 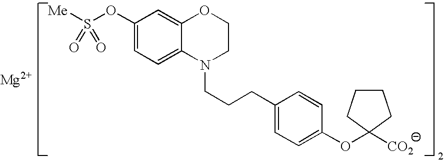 | 1-[4-{3-(7-Methanesulfonyloxy-3, 4-dihydro-2H-bezo[b][1,4]yl)butyl}phenoxy]cyclopentane-1- carboxylic acid, magnesium salt |
| 60. | 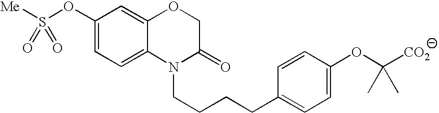 | 2-Methyl-2-[4-{4-(7- methanesulfonyloxy-3,4-dihydro- 2H-bezo[b][1,4]oxazin-3-on-4- yl)butyl}phenoxy]propanoic acid, Arginine salt |
| | 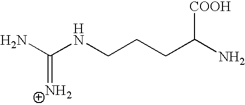 | |

Another embodiment of the present invention is a compound of formula (If) where “Ar₁” is substituted with —OSO₂R³, wherein R³ is as defined above preferably optionally substituted groups selected from alkyl or aryl.

And all other symbols are as defined above.

Compounds of the present invention are agonists or peroxisome proliferators activated receptor (PPAR) and hence are useful for the treatment or prophylaxis of patients suffering from a condition caused by the non activation of PPAR, who are in need of such therapy. Pharmacologically effective amounts of the compounds, including pharmaceutically acceptable salts thereof, are administered to the patient to inhibit insulin resistance and dyslipidemia such as diabetes, hypertension, coronary heart disease, atherosclerosis, stroke, peripheral vascular diseases, psoriasis, polycystic ovarian syndrome (PCOS), inflammatory bowel diseases, osteoporosis, myotonic dystrophy, pancreatitis, retinopathy, arteriosclerosis, xanthoma and related disorders.

The compounds of the present invention are administered in dosages effective to agonize peroxisome proliferators activated receptor where such treatment is needed, as, for example, in the prevention or treatment of diabetes, hypertension, coronary heart disease, atherosclerosis, stroke, peripheral vascular diseases, psoriasis, polycystic ovarian syndrome (PCOS), inflammatory bowel diseases, osteoporosis, myotonic dystrophy, pancreatitis, retinopathy, arteriosclerosis, xanthoma and related disorders. For use in medicine, the salts of the compounds of this invention refer to non-toxic “pharmaceutically acceptable salts.” Other salts may, however, be useful in the preparation of the compounds according to the invention or of their pharmaceutically acceptable salts. Salts encompassed within the term “pharmaceutically acceptable salts” refer to non-toxic salts of the compounds of this invention which are generally prepared by reacting the free acid with a suitable organic or inorganic base. Representative salts include the following:

Li, Na, K, Ca, Mg, Fe, Cu, Zn, Mn; N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, dialkylamine, trialkylamine, thiamine, aminopyrimidine, aminopyridine, purine, spermidine; alkylphenylamine, glycinol, phenyl glycinol; glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids; D-isomers or substituted amino acids; guanidine, substituted guanidine wherein the substituents are selected from nitro, amino, alkyl, alkenyl, alkynyl, ammonium or substituted ammonium salts and aluminum salts; sulphates, nitrates, phosphates, perchlorates, borates, hydrohalides, acetates, tartrates, maleates, citrates, succinates, palmoates, methanesulphonates, benzoates, salicylates, hydroxynaphthoates, benzenesulfonates, ascorbates, glycerophosphates, or ketoglutarates.

The compounds of the present invention, may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention; further included are all mixtures of the two enantiomers. Also included within the scope of the invention are polymorphs as well as hydrates of the compounds of the instant invention.

The present invention includes within its scope prodrugs of the compounds of this invention. In general, such pro drugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

Definitions:

The terms “individual,” “subject,” “host,” and “patient” refer to any subject for whom diagnosis, treatment, or therapy is desired. In one embodiment, the individual, subject, host, or patient is a human. Other subjects may include, but are not limited to, animals including but not limited to, cattle, sheep, horses, dogs, cats, guinea pigs, rabbits, rats, primates, opossums and mice. Other subjects include species of bacteria, phages, cell cultures, viruses, plants and other eucaryotes, prokaryotes and unclassified organisms.

The terms “treatment,” “treating,” “treat,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The term “therapeutically effective amount” shall mean that amount of a drug or pharmaceutical agent that will elicit the biological or medical response of a tissue, system or patient that is being sought by a researcher.

“halo” is iodine, bromine, chlorine or fluorine.

The terms “polycyclic” or “polycyclyl,” as used herein, refer to unsubstituted or substituted fused or bridged polycyclic systems containing from 7 to 20 carbon atoms and which can contain one or more degrees of unsaturation. Preferably, the term “polycyclyl” refers to unsubstituted or substituted fused or bridged bi- or tricyclic systems containing from 7-15 carbon atoms and which are saturated or can contain one or six degrees of unsaturation. More preferably, the term “polycyclyl” refers to unsubstituted or substituted fused or bridged bi- or tri-cyclic systems containing from 8-12 carbon atoms and which can contain upto six degrees of unsaturation. Examples of preferred polycyclyl systems include, but are not limited to, naphthalene, tetraline, dihydro naphthalene, decahydronaphthalene, quinoline, tetrahydro quinoline, iso quinoline, tetrahydro isoquinoline, quinazolinone, benzoxazine, dihydrobenzoxazine, benzothiazine, dihydrobenzothiazine, indole, dihydro indole, isoindole, dihydro isoindole, pyrrolo oxazole, pyrrolizidine, benzotriazole, benzoxazole, benzothiazole, imidazopyridazine, pyrazolopyrimidine, pyrazolopyridine, benzimidazole, indazole, furopyridine, benzofuran, benzothiophene, pyrindine, pyrazolodiazepine, benzotriazene, azirinoindole, pyrazoloquinoline, imidazoquinoline, benzothiazene, phthalazene, quinazoline, quinoxaline, benzoxathiin, carbazole, naphthofuran, naphthopyrans, benzothiophene, acridine, benzoisoquinoline, benzoquinoline.

‘Alkyl’ group is a linear or branched (C₁-C₁₀)alkyl group. Exemplary alkyl groups include methyl, ethyl, n-propyl, iso-propyl, n-butyl, iso-butyl, t-butyl, n-pentyl, iso-pentyl, hexyl, heptyl, octyl and the like.

‘Alkenyl’ is a (C₂-C₁₀)alkenyl group. Exemplary alkenyl groups include ethenyl, propenyl, prop-1-enyl, isopropenyl, butenyl, but-1-enyl, isobutenyl, pentenyl, pent-1-enyl, hexenyl, pent-2-enyl, 2-methyl-but-2-ene, 2-methyl-pent-2-nyl and the like

‘Alkynyl’ is (C₂-C₁₀)alkynyl. Exemplary alkynyl groups include ethenyl, propynyl, prop-1-ynyl, butynyl, but-ynyl and the like.

“cycloalkyl” is (C₃-C₈)cycloalkyl group. Exemplary cycloalkyl groups include, but are not limited to cyclopropyl, cyclobutyl, cyclopentyl, cyclohexyl and the like.

“alkoxy” is (C₁-C₁₀)alkyl-O—, wherein (C₁-C₁₀)alkyl group is as defined above. Exemplary alkoxy groups include but are not limited to methoxy, ethoxy, propyloxy, butyloxy, iso-propyloxy and the like.

“thioalkoxy” is (C₁-C₁₀)alkyl-S—, wherein (C₁-C₁₀)alkyl group is as defined above. Exemplary alkoxy groups include but are not limited to thiomethoxy, thioethoxy, thiopropyloxy, thiobutyloxy, thioiso-propyloxy and the like.

“hydroxyalkyl” is (C₁-C₁₀)alkyl-OH, wherein (C₁-C₁₀)alkyl group is as defined above. Exemplary hydroxyalkyl groups include but are not limited to hydroxy methyl, hydroxyethyl, hydroxypropyl, hydroxyisopropyl, hydroxybutyl, hydroxyisobutyl, hydroxyter. butyl and the like.

“heterocyclyl” is a non-aromatic saturated monocyclic or multicyclic ring system of about 5 to about 10 carbon atoms, having at least one hetero atom selected from O, S or N. Exemplary heterocyclyl groups include, but are not limited to aziridinyl, pyrrolidinyl, piperidinyl, piperazinyl, morpholinyl, thiomorpholinyl, thiazolidinyl, 1,3-dioxolanyl, 1,4-dioxanyl and the like.

‘Aryl’ is optionally substituted monocyclic or polycyclic ring system of about 6 to 14 carbon atoms. Exemplary groups include phenyl, naphthyl and the like.

‘Heteroaryl’ is an aromatic monocyclic or polycyclic ring system of about 5 to about 10 carbon atoms, having at least one heteroatom selected from O, S or N. Exemplary heteroaryl groups include as pyrazinyl, isothiazolyl, oxazolyl, pyrazolyl, pyrrolyl, pyridazinyl, thienopyrimidyl, furanyl, indolyl, isoindolyl, benzo[1,3]dioxolyl, 1,3-benzoxathiole, quinazolinyl, pyridyl, thiophenyl and the like.

“haloalkoxy” is halo substituted (C₁-C₁₀)alkyl-O—, wherein (C₁-C₁₀)alkyl group is as defined above. Exemplary haloalkoxy groups include but are not limited to trifluoromethoxy, 1,2-dichloroethoxy and the like.

‘Haloalkyl’ is halo-(C₁-C₁₀)alkyl, where halo and (C₁-C₁₀)alkyl are as define above. Exemplary haloalkyl groups include fluoromethyl, chloromethyl, fluoroethyl, chloroethyl, trifluoromethyl and the like.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

It is to be understood that this invention is not limited to the particular methodology, protocols, cell lines, constructs, and reagents described herein and as such may vary. It is also to be understood that the terminology used herein is for the purpose of describing particular embodiments only, and is not intended to limit the scope of the present invention which will be limited only by the appended claims.

The dosage regimen utilizing. the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or betalactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of formula (I) can generally be prepared, for example in the course of a convergent synthesis, by linkage of two or more fragments which can be derived retrosynthetically from the formula (I). in the preparation of compounds of formula (I), it may be generally necessary in the course of synthesis temporarily block functional groups which could lead to undesired reactions or side reactions in a synthetic step by protective group suited to the synthesis problem and known to the person skilled in the art. The method of fragment coupling is not restricted to the following examples, but is generally applicable for synthesis of compounds of formula (I).

The novel compounds of the present invention were prepared according to the procedure of the following schemes and examples, using appropriate materials and are further exemplified by the following specific examples. The most preferred compounds of the invention are any or all of those specifically set forth in these examples. These compounds are not, however, to be construed as forming the only genus that is considered as the invention, and any combination of the compounds or their moieties may itself form a genus. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

The following Schemes and Examples describe procedures for making representative compounds of the present invention. Moreover, by utilizing the procedures described in detail, one of ordinary skill in the art can readily prepare additional compounds of the present invention claimed herein.
Route 1:

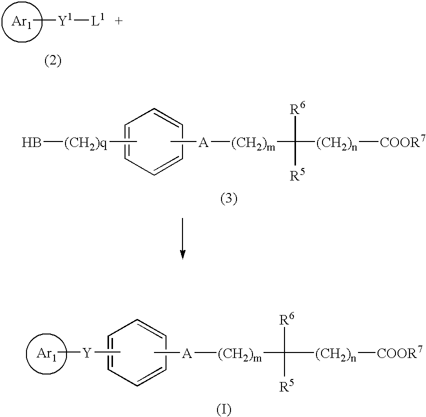

Reaction of compound of formula (2), where Y¹ represents (CH₂)ₚ, (CH₂)ᵣB(CH₂)_{q}, L¹ represents a leaving group selected from halo or mesyloxy and “Ar₁” is as defined, with a compound of formula (3), wherein the all the symbols are as defined, to produce a compound of the formula (I), wherein Y represents (CH₂)ₚB(CH₂)_{q}, (CH₂)ᵣB(CH₂)ₚD(CH₂)_{q} and all other symbols are as defined above, may be carried out in the presence of a solvent such as diethyl ether, THF, DMF, DMSO, DME, toluene, benzene, acetone, acetonitrile and the like or a mixture thereof. The reaction may be carried out in an inert atmosphere, which may be maintained by using inert gases such as N₂, Ar, He and the like. The reaction may be effected in the presence of a base such as K₂CO₃, Na₂CO₃ or NaH or mixtures thereof. The reaction temperature may range from −20° C.-120° C., preferably at a temperature in the range of 0° C.-120° C. The duration of the reaction may range from 1 to 48 hours. Phase transfer catalyst such as tetraalkylammonium halides or hydroxides or bisulphates may be employed.

Alternatively, when L¹=OH and B=Oxygen, Mitsunobu reaction conditions may be employed to obtain compound of formula (I)

The intermediate (2) may be obtained by reacting “Ar₁” which as defined, with (2a) 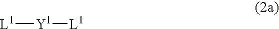
where Y¹ represents (CH₂)ₚ, (CH₂)ᵣB(CH₂)_{q}, L¹ represents a leaving group selected from halo or mesyloxy in the presence of a solvent such as diethyl ether, THF, DMF, DMSO, DME, toluene, benzene, acetone, acetonitrile and the like or a mixture thereof and a base such as KOH, K₂CO₃, Na₂CO₃ or NaH. The reaction may be carried out in an inert atmosphere, which may be maintained by using inert gases such as N₂, Ar, He and the like.
Alternatively, the intermediate (2) where Y¹ is (CH₂)ᵣB(CH₂)ₚ and L¹ represents a leaving group selected from halo or mesyloxy may be obtained by reacting the compound of formula (2b) 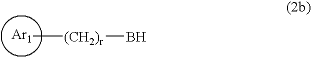
wherein “Ar₁” and B have the meaning as described, with (2c) 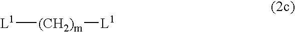
where L¹ represents a leaving group selected from halo or mesyloxy in the presence of a solvent such as diethyl ether, THF, DMF, DMSO, DME, toluene, benzene, acetone, acetonitrile and the like or a mixture thereof and a base such as K₂CO₃, Na₂CO₃ or NaH. The reaction may be carried out in an inert atmosphere, which may be maintained by using inert gases such as N₂, Ar, He and the like. The reaction temperature may range from −20° C.-120° C., preferably at a temperature in the range of 0° C.-120° C. The duration of the reaction may range from 1 to 48 hours. Phase transfer catalyst such as tetraalkylammonium halides or hydroxides or bisulphates may be employed.
Route 2:

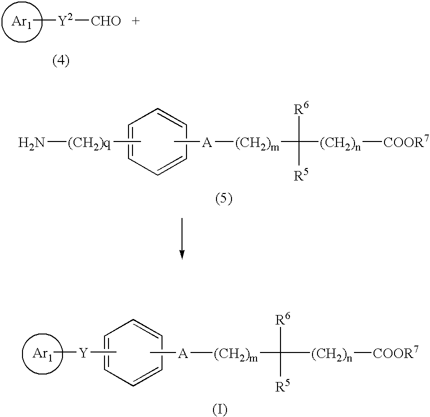

Reaction of compound of formula (4), where Y² represents (CH₂)_{P-1} and “Ar₁” is as defined with a compound of formula (5), where all other symbols are as described, to produce a compound of the formula (I), wherein Y represents (CH₂)ₚB(CH₂)_{q} where B represents NH and all other symbols are as defined above, may be carried out in two steps; the first step being the imine formation, followed by reduction. Formation of imine may be carried out in solvents such as benzene, toluene, chloroform, dichloromethane, MeOH, EtOH, i-PrOH and the like. The reaction may be effected in the presence of a catalyst such as pTsOH, NaOAc, BF₃.OEt, KOAc and the like or the mixtures thereof. The temperature of reaction may range from room temperature to the reflux temperature of the solvent used. The reaction time may be 2 h to 24 h, preferably in the range 2 h to 12 h.

The imine can also be obtained by the reaction of a compound of general formula (4) with a compound of formula (5) using solvent such as CH₂Cl₂, CHCl₃, chlorobenzene, benzene, THF, in the presence of catalyst such as p-toluenesulfonic acid, methanesulfonic acid, TFA, TfOH, BF₃—OEt₂ and the like. The reaction may also be carried out in presence of activated molecular sieves. The temperature of the reaction may range from 10° C. to 100° C., preferably at a temperature in the range from 10° C. to 60° C. The reaction time may range from 1 h to 48 h.

The imine product thus obtained above may be reduced by using Na(CN)₃H₃—HCl (ref: Hutchins, R. O. et al. J. Org. Chem. 1983, 48, 3433), NaBH₄, H₂—Pd]/C, H₂—Pt/C, H₂—Rh/C and the like in solvents such as methanol, ethanol and the like.
Route 3:

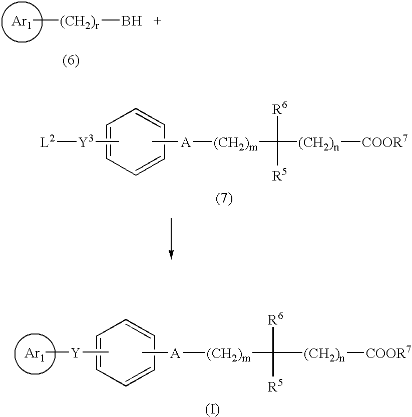

Reaction of compound of formula (6), wherein all symbols are as defined with a compound of formula (7) Y³ represents (CH₂)ₚ, (CH₂)ₚB(CH₂)_{q}, L² represents a leaving group selected from halo or mesyloxy, Ar₂ and Z have the meaning as described to produce a compound of the formula (I), wherein Y represents (CH₂)ₚB(CH₂)_{q}, (CH₂)ᵣB(CH₂)ₚB(CH₂)_{q} and all other symbols are as defined above, may be carried out in the presence of aprotic solvents such as diethyl ether, THF, DMF, DMSO, DME, toluene, benzene, acetone, acetonitrile and the like or mixtures thereof. The reaction may be carried out in an inert atmosphere, which may be maintained by using inert gases such as N₂, Ar, He and the like. The reaction may be effected in the presence of a base such as K₂CO₃, Na₂CO₃ or NaH or mixtures thereof. The reaction temperature may range from −20° C.-120° C., preferably at a temperature in the range of 0° C.-120° C. The duration of the reaction may range from 1 to 48 hours. Phase transfer catalyst such as tetraalkylammonium halides or hydroxides or bisulphates may be employed.

Alternatively, when L²=OH and B=Oxygen, Mitsunobu reaction conditions may be employed to obtain compound of formula (I)

The intermediate (6) wherein “Ar₁” is substituted by mesyloxy may be obtained by mesylating the corresponding hydroxy substituted intermediate (6a) 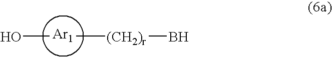
with mesyl chloride in the presence of a base such as trialkylamine, pyridine or K₂CO₃ and solvent such as chloroform, dichloromethane or THF at a temperature range of −25° C. to room temperature, preferably 0° C. to room temperature.
Route 4:

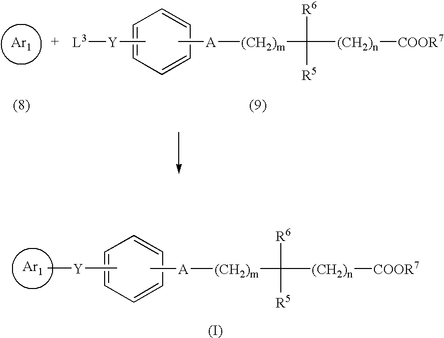

Reaction of compound of formula (8), wherein “Ar₁” has the meaning as described with a compound of formula (9), where Y represents (CH₂)ₚ, (CH₂)ₚB(CH₂)_{q}, (CH₂)ᵣB(CH₂)ₚB(CH₂)_{q}, L³ represents a leaving group selected from halo or mesyloxy, and all other symbols have the meaning as described to produce a compound of the formula (I) wherein Y represents (CH₂)ₚ, (CH₂)ₚB(CH₂)_{q}, (CH₂)ᵣB(CH₂)ₚB(CH₂)_{q}, and all other symbols are as defined above, may be carried out in the presence of aprotic solvents such as diethyl ether, THF, DMF, DMSO, DME, toluene, benzene, acetone, acetonitrile and the like or mixtures thereof. The reaction may be carried out in an inert atmosphere, which may be maintained by using inert gases such as N₂, Ar, He and the like. The reaction may be effected in the presence of a base such as KOH, K₂CO₃, Na₂CO₃ or NaH or mixtures thereof. The reaction temperature may range from −20° C.-120° C., preferably at a temperature in the range of 0° C.-120° C. The duration of the reaction may range from 1 to 48 hours. Phase transfer catalyst such as tetraalkylammonium halides or hydroxides or bisulphates may be employed.
Route 5:

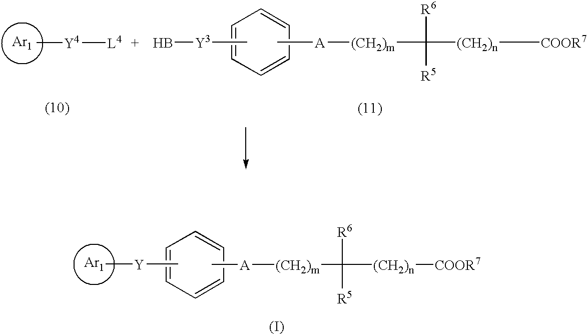

Reaction of compound of formula (10), where Y⁴ represents (CH₂)ₚ, (CH₂)ₚB(CH₂)_{q}, L⁴ represents a leaving group selected from halo or mesyloxy, “Ar₁” has the meaning described, with a compound of formula (11), where Y³ represents (CH₂)_{q} and all other symbols are as described to produce a compound of the formula (I) wherein Y represents (CH₂)ₚB(CH₂)_{q} and all other symbols are as defined above, may be carried out in an inert atmosphere, which may be maintained by using inert gases such as N₂, Ar, He and the like. The reaction may be effected in the presence of a base such as NaH and a solvent such as DMF, THF, dioxane, ether or a mixture thereof. The reaction temperature may range from −20° C.-120° C., preferably at a temperature in the range of 0° C.-120° C. The duration of the reaction may range from 1 to 48 hours. Phase transfer catalyst such as tetraalkylammonium halides or hydroxides or bisulphates may be employed.
Alternatively, when L⁴=OH and B=Oxygen, Mitsunobu reaction conditions may be employed to obtain compound of formula (I)

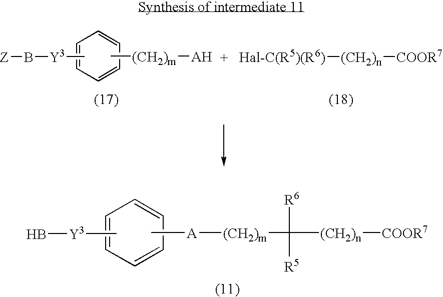

Reaction of compound of formula (17) wherein Z is protecting groups like benzyl, THP, TBDMS and likes, and all symbols are as defined above, with compound of formula (18) where all symbols are as defined above to produce a compound of formula (11) where all symbols are as defined above may be carried out in the presence of an aprotic solvent such as THF, DMF, DMSO, DME, toluene, benzene, xylene, acetonitrile and the like or mixtures thereof. The reaction may be carried out in the presence of an organic base such as triethylamine, collidine, lutidine and the like or mixtures thereof. The reaction may be carried out in an inert atmosphere that may be maintained by using an inert gas such as nitrogen, helium or argon. The reaction may be effected in the presence of a base such as K₂CO₃, Na₂CO₃, NaNH₂, n-BuLi, NaH, KH and the like. The reaction temperature may range from 0 to 120° C., preferably in the range of 25 to 100° C. The duration of the reaction may range from 1 to 72 h, preferably from 1 to 24 h.

Alternatively, Mitsunobu reaction conditions may be employed to obtain compound of formula (I)

Route 6:

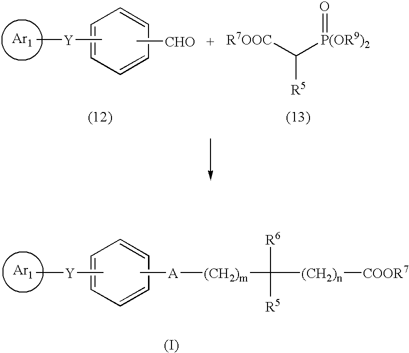

Reaction of compound of formula (12), where all symbols have the meaning described, with modified Wittig reagent (13), where R⁷ represents substituted or unsubstituted groups selected from alkyl, cycloalkyl, R⁵ represents (C₁₋₁₂)alkoxy, R⁹ represents (C₁₋₆)alkyl to produce a compound of formula (I) wherein A and R⁶ together represent a bond, R⁵ represents (C₁₋₁₂)alkoxy, m and n is 0 and R⁷ represents substituted or unsubstituted groups selected from (C₁₋₁₂)alkyl, cycloalkyl, and all other symbols are as defined above, may be carried out in the presence of a base such as alkali metal hydrides like NaH or KH; organolithiums such as CH₃Li, BuLi, LDA, TMEDA and the like; alkoxides such as NaOMe, NaOEt, K⁺BuO⁻ and the like or mixtures thereof. The reaction may be carried out in the presence of solvents such as diethyl ether, THF, dioxane, DMF, DMSO, DME, toluene, benzene and the like or mixtures thereof. HMPA may be used as cosolvent. The reaction temperature may range from −78° to 50° C., preferably at a temperature in the range of −10° C. to 30° C. The reaction is more effective under anhydrous conditions.

Alternatively, the compound of formula (I) may be prepared by reacting the compound of formula (12) where all symbols are as defined earlier with Wittig reagents such as Hal Ph₃P⁺CH—R⁷)CO₂R⁹ under similar reaction conditions as described above.

Route 7:

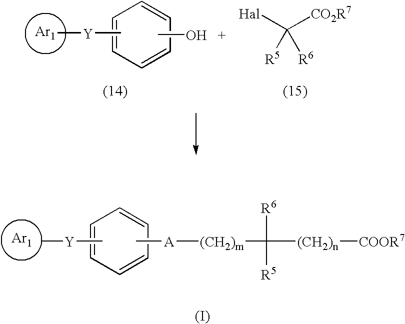

Reaction of compound of formula (14), where all symbols have the meaning described with compound of formula (15), where R⁵, R⁶ and R⁷ are as described above; to produce a compound of formula (I) wherein A represents oxygen, R⁵, R⁶ and R⁷ are as described above, may be carried out in the presence of an aprotic solvent such as THF, DMF, DMSO, DME, toluene, benzene, xylene, acetonitrile and the like or mixtures thereof. The reaction may be carried out in the presence of an organic base such as triethylamine, collidine, lutidine and the like or mixtures thereof. The reaction may be carried out in an inert atmosphere that may be maintained by using an inert gas such as nitrogen, helium or argon. The reaction may be effected in the presence of a base such as K₂CO₃, Na₂CO₃, NaNH₂, n-BuLi, NaH, KH and the like. The reaction temperature may range from 0 to 120° C., preferably in the range of 25 to 100° C. The duration of the reaction may range from 1 to 72 h, preferably from 1 to 24 h.

Alternatively, Mitsunobu reaction conditions may be employed to obtain compound of formula (I)

Route 8:

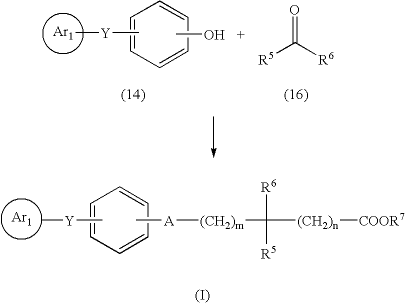

Reaction of a compound of formula (14), where all symbols have the meaning described with a compound of formula (16), where R⁵ and R⁶ are as defined above to produce a compound of formula (I), where A represents oxygen, R⁵ and R⁶ are as defined above; m and n is 0 and R⁷ represents hydrogen, may be carried out in the presence of chloroform-NaOH or chloroform-KOH and a solvent such as THF, dioxane, ethylether, benzene, toluene and the like or a mixture thereof at a temperature range −25° C. to room temperature preferably O° C. to room temperature. (ref. JMC, 2000, 43, 4726-4737. Chem Pharm Bull, 2000, 48, 1978-1985)

Route 9:

The compound of formula (I) where R⁴ represent alkyl, alkenyl, —S(O—R⁸ or —C(O)R⁸ where R⁸ is alkyl, alkoxy is obtained by reacting a compound of formula (I) where Y represents (CH₂)ₚNR⁴(CH₂)_{q} and R⁴ represents hydrogen, by reacting with R⁸SO₂Cl, R⁸C(O)Cl or an acid anhydride in the presence of a base selected from trialkylamine, pyridine or K₂CO₃ and solvent such as chloroform, dichloromethane or THF at a temperature range of −25° C. to room temperature, preferably 0° C. to room temperature. Catalytic amounts of DMAP may also be used to accelerate the reaction. 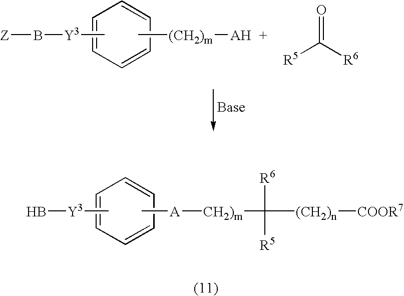
Z is protecting groups like benzyl, THP, TBDMS and likes.
Definition and reaction condition is like Route-8

The present invention is further illustrated by the following examples, which are not to be construed in any way as imposing limitations upon the scope thereof, but rather are illustrative only. On the contrary, it is to be clearly understood that resort may be had to various other embodiments, modifications, and equivalents thereof which, after reading the description herein, may suggest themselves to one of ordinary skill in the art without departing from the spirit of the present invention.

The following acronyms, abbreviations, terms and definitions have been used throughout the experimental section. Acronyms or abbreviations: TLC (thin layer chromatography), mL (milli liters), mp (melting point), RT (room temperature, 20-45° C.), aq (aqueous), min (minute), h (hr, hour), atm (atmosphere), conc. (concentrated), MS (mass spectroscopy/spectrometry), NMR (nuclear magnetic resonance). NMR abbreviations: br (broad), apt (apparent), s (singlet), d (doublet), t (triplet), q (quartet), dq (doublet of quartets), dd (doublet of doublets), dt (doublet of triplets), m (multiplet).

### Preparation 1

### 6-methanesulfonyloxynapthyl-2-carboxaldehyde

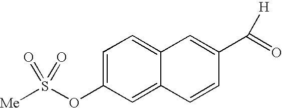

### Step 1: Methyl-6-methanesulfonyloxy β-napthoate

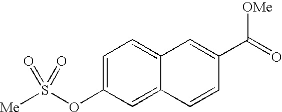

To a mixture of methyl 6-hydroxy β-napthoate (5.0 gm, 1.0 eq, 24.75 mmol) and Et₃N (8.6 mL, 2.5 eq, 61.88 mmol) in dry. DCM (125 mL) stirred at 0° C., methanesulfonylchloride (2.89 mL, 1.5 eq, 37.12 mmol) was added and stirring was continued for 5 hr. The reaction mixture was diluted with 200 mL of DCM and washed with aqueous citric acid followed by water and brine. Organic layer was dried (Na₂SO₄), condensed, and the residue was chromatographed using ethyl acetate and hexane to obtain the title compound as white solid (6 gm, 86% yield). Mp: 106-108° C.

¹H NMR (CDCl₃, 200 MHz) δ: 3.23 (s, 3H); 3.99 (s, 3H); 7.47 (dd, J=9.4, 2.4 Hz, 1H); 7.81 (d, J=2.4 Hz, 1H); 7.89 (d, J=8.8 Hz, 3H); 8.02 (d, J=8.8 Hz, 1H); 8.13 (dd, J=8.8 Hz, 1.4 Hz, 1H); 8.63 (s, 1H).

Mass m/z (ES): 281.1[M+1], 298.1 [M+NH₄^{+], 303.0} [M+Na], 578.3 [M₂+NH₄^{+], 583.3} [M₂+Na].

### Step 2: 6-(Methanesulfonyloxy)napth-2-ylmethyl alcohol

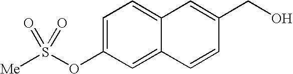

A solution of methyl-6-methanesulfonyloxy β-napthoate (6 gm, 1 eq, 21.4 mmol) obtained in step 1 of preparation 1, in dry THF (107 mL) was cooled up to −70° C., and then DIBAL (53 mL, 3 eq, 64.2 mmol) was added drop wise with constant stirring at −70° C. After the addition, the reaction mixture was slowly allowed to attain RT (4 hr). Reaction mixture was quenched with Methanol (150 mL), followed by the addition of saturated solution of Na₂SO₄. Finally reaction mixture was filtered through celite. Filterate was dried (Na₂SO₄), condensed, and the residue was chromatographed using ethyl acetate and hexane to obtain the title compound as white solid (2.9 gm, 53% yield). Mp: 96-98° C.

¹H NMR (CDCl₃, 200 MHz) δ: 3.19 (s, 3H); 4.87(s, 2H); 7.40(dd, J=9.2, 2.4 Hz, 1H); 7.54 (d, J=8.8 Hz, 1H); 7.75(d, J=2 Hz, 1H); 7.81-7.89 (aromatics, 3H)

IR (neat) cm⁻¹:

Mass m/z (ES): 270.3 [M+NH₄⁺], 275.3 [M+Na], 522.5 [M₂+NH₄⁺].

### Step 3: 6-(Methanesulfonyloxy)napthyl-2-carboxaldehyde

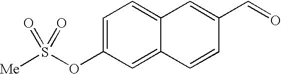

To a stirred solution of 6-methanesulfonyloxynapth-2-ylmethyl alcohol (2.9 gm, 1 eq, 1.51 mmol) obtained in step 2 of preparation 1 and activated molecular sieves (4 A) in dry DCM (60 mL), pyridiniumdichromate (4.75 gm, 1.1 eq, 12.65 mmol) was added at 0° C. After the addition, the reaction mixture was allowed to stir at RT for 15 hr. Reaction mixture was filtered through celite, filtrate was dried (Na₂SO₄), condensed, and the residue was chromatographed using ethyl acetate and hexane to obtain the title compound as white solid (1.2 gm, 41% yield). Mp: 90-92° C.

¹H NMR (CDCl₃, 200 MHz) δ: 3.25 (s, 3H); 7.52 (dd, J=8.8, 2.5 Hz, 1H); 7.83 (d, J=2 Hz, 1H); 7.92-8.10 (aromatics, 3H); 8.37 (s, 1H); 10.17 (s, 1H).

IR (neat) cm⁻¹: 2932, 1681, 1624, and 1469.

Mass m/z(CI): 251 [M+1].

### Preparation 2

### 6-(Methanesulfonyloxy)napth-2-ylmethyl bromide

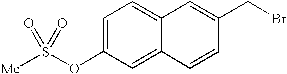

A mixture of 6-methanesulfonyloxynapth-2-ylmethanol (2 gm, 1 eq, 7.9 mmol) obtained in step 2 of preparation 1, CBr₄ (2.88 gm, 1.1 eq, 8.69 mmol) and PPh₃ (3.10 gm, 1.5 eq, 11.85 mmol) in dry THF (40 mL) was stirred at RT for 17 h. Reaction mixture was condensed and diluted with ethyl acetate (100 mL) and washed with water. Organic layer was dried (Na₂SO₄), condensed, and the residue was chromatographed using ethyl acetate and hexane to obtain the title compound as white solid (770 mg, 31% yield). Mpt: 100-102° C.

¹H NMR (CDCl₃, 200 MHz) δ: 3.19 (s, 3H); 4.65 (s, 2H); 7.42 (dd, J=9, 2.4 Hz, 1H); 7.57 (dd, J=8.4, 1.4 Hz, 1H); 7.75 (d, J=2.2 Hz, 1H); 7.82-7.90 (aromatics, 3H)

IR (neat) cm⁻¹: 2925, 1360, and 1173.

Mass m/z(CI): 315 [M (⁷⁹Br)+13, 317 [M (⁸¹Br)+1]

### Preparation 3

### 1,2,3,4-Tetrahydro-6-(methanesulfonyloxy napth-2-ylmethyl methanesulfonate

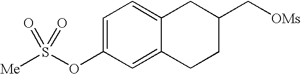

### Step 1: Ethyl-benzyloxy-1,2,3,4-tetrahydro-1-oxo-βnapthoate

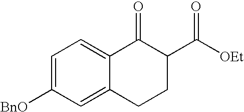

To a suspension of NaH (816 mg, 60% in oil, 2 eq, 20.42 mmol) in 40 mL dry THF, diethylcarbonate (3.7 mL, 3 eq, 30.64 mmol) was added, and the mixture was heated at 60° C. To that a solution of 6-(benzyloxy)tetralone (2.57 g, 1 eq, 10.21 mmol) in 10 mL THF was added and the heating was continued for another 4 hours. Reaction mixture was condensed and diluted with ethyl acetate (100 mL) and washed with water. Organic layer was dried (Na₂SO₄), condensed, and the residue was chromatographed using ethyl acetate and hexane to obtain the title compound as thick liquid (2.58 g, 78% yield). TLC as well as ¹H-NMR indicates that the compound is a mixture keto/enol tautomers of 70:30 ratio. For clarification, ¹H-NMR data is given here for the keto form.

¹H NMR (CDCl₃, 400 MHz) δ: 1.28 (t, J=7 Hz, 3H); 2.30-3.10 (m, 4H); 3.54 (dd, J=10, 4.5 Hz, 1H); 4.23 (q, J=7 Hz, 2H); 5.11 (s, 2H); 6.77-6.92 (aromatics, 2H); 7.32-7.44 (aromatics, 5H); 8.02 (d, J=8.6 Hz, 1H).

IR (neat) cm⁻¹: 2936, 1737, 1677, and 1600.

Mass m/z(CI): 325 [M+1].

### Step 2: Ethyl-hydroxy-1,2,3,4-tetrahydro-β-napthoate

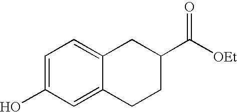

Ethyl-6-benzyloxy-1,2,3,4-tetrahydro-1-oxo-β-napthoate (460 mg, 1.42 mmol) was hydrogenated under H₂ (5 psi pressure) at RT for 6-7 h using 10%-Pd/C (285 mg) as catalyst in a combination of solvents EtOH (14 mL)/water (1.4 mL)/conc. HCl (365 μL) to obtain the desired compound as white solid (250 mg, 80% yield) after usual workup and purification through column chromatography (ethyl acetate/hexane). Mp: 80-82° C.

¹H NMR (CDCl₃, 400 MHz) δ: 1.28 (t, J=7.2 Hz, 3H); 1.78-1.85 (m, 1H); 2.15-2.22 (m, 1H); 2.65-2.72 (m, 1H); 2.78-2.82 (m, 2H); 2.85-2.95 (m, 2H); 4.17 (q, J=7.2 Hz, 2H); 4.64 (s, 2H); 6.55-6.62 (aromatics, 2H); 6.95 (d, J=8 Hz, 1H).

IR (neat) cm⁻¹: 3397, 2934, 1737, 1707, and 1611.

Mass m/z(CI): 3221 [M+1].

### Step 3: 6-Hydroxy-1,2,3,4-tetrahydronapth-2-ylmethyl alcohol

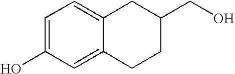

A solution of ethyl-6-hydroxy-1,2,3,4-tetrahydro-β-napthoate (480 mg, 1 eq, 2.184 mmol) obtained in step 2 of preparation 3, in dry THF (22 mL) was cooled up to −70° C., and then DIBAL (10.8 mL, 6 eq, 13.1 mmol) was added drop wise with constant stirring at −70° C. After the addition, the reaction mixture was slowly allowed to attain RT (4 hr). Reaction mixture was quenched with methanol (40 mL), followed by the addition of saturated solution of Na₂SO₄. Finally reaction mixture was filtered through celite. Filtrate was dried (Na₂SO₄), condensed, and the residue, as a crude, was directly used for next reaction.

### Step 4: 1,2,3,4-Tetrahydro-6-(methanesulfonyloxy)-napth-2-ylmethyl methanesulfonate

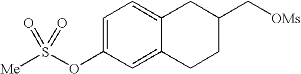

To a stirred solution of 6-Hydroxy-1,2,3,4-tetrahydronapth-2-ylmethyl alcohol (280 mg, 1 eq, 1.36 mmol) obtained in step 3 of preparation 3, and Et₃N (1.3 mL, 6 eq, 8.15 mmol) in dry DCM (7 mL) at 0° C., methanesulfonylchloride (0.316 mL, 3 eq, 4.07 mmol) was added and stirring was continued for 5 h. The reaction mixture was diluted with 50 mL of DCM and washed with citric acid solution followed by water and brine. Organic layer was dried (Na₂SO₄), condensed, and the residue was chromatographed using ethyl acetate and hexane to obtain the title compound as thick mass (430 mg, 95% yield).

¹H NMR (CDCl₃, 400 MHz) δ: 1.45-1.55 (m, 1H); 1.2.00-2.06 (m, 1H); 2.20-2.30 (m, 1H); 2.56 (dd, J=16, 10 Hz, 1H); 2.84-3.03 (m 3H); 3.04 (s, 3H); 3.13 (s, 3H); 4.18-4.25 (m, 2H); 7.00-7.05 (aromatics, 2H); 7.10-7.13 (aromatics, 1H).

IR (neat) cm⁻¹: 2937, 1352, 1173.

Mass m/z (CI): 335 [M+1]

### Preparation 4

### 6-benzyloxynapthyl-2-carboxaldehyde

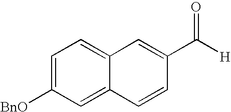

### Step 1: Methylbenzyloxy-β-napthoate

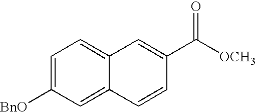

A mixture of Methyl-6-hydroxy-β-napthoate (6 g, 1 eq, 29.70 mmol), benzyl bromide (3.9 mL), and anhydrous K₂CO₃ (8.2 g, 2 eq, 59.41 mmol) in dry DMF was stirred at RT for 16 hr. Reaction mixture was diluted with ethyl acetate (200 mL) and washed with water (3×100 mL). Organic layer was dried (Na₂SO₄), condensed, and the residue was chromatographed using ethyl acetate and hexane to obtain the title compound as white solid (8.4 g, 98% yield). Mp: 149-151° C.

¹H NMR (CDCl₃, 200 MHz) δ: 3.97 (s, 3H); 5.21(s, 2H); 7.30-7.48 (aromatics, 7H); 7.75 (d, J=8.6 Hz, 1H); 7.87 (d, J=8.6 Hz, 1H); 8.03 (d, J=8.6 Hz, 1H); 8.54 (s, 1H).

IR (neat) cm⁻¹: 3437, 2924, 1716, and 1624.

Mass m/z (CI): 293 [M+1].

### Step 2: 6-Benzyloxynapth-2-ylmethyl alcohol

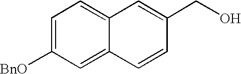

A solution of Methyl-6-benzyloxy-β-napthoate (8 g, 1 eq, 27.39 mmol) obtained) in step 1 of preparation 4, in dry THF (200 mL) was cooled up to −70° C., and then DIBAL (68 mL, 3 eq, 82.19 mmol) was added drop wise with constant stirring at −70° C. After the addition, the reaction mixture was slowly allowed to attain RT (5 h). Reaction mixture was quenched with Methanol (250 mL), followed by the addition of saturated solution of Na₂SO₄. Finally reaction mixture was filtered through celite. Filtrate was dried (Na₂SO₄), condensed, and the residue was chromatographed using ethyl acetate and hexane to obtain the title compound as white solid (7.1 g, 98% yield). Mp: 130-132° C.

¹H NMR (CDCl₃, 200 MHz) δ: 1.71 (t, J=5.8 Hz, OH); 4.82 (d, J=5.8 Hz, 2H); 5.18(s, 2H); 7.24 (d, J=7.4 Hz, 2H); 7.34-7.51(aromatics, 6H); 7.71-7.77 (aromatics, 3H)

IR (neat) cm⁻¹: 2924, 1694, and 1617.

Mass m/z (CI): 265 [M+1], 264 [M], 247 [M-OH].

### Step 3: 6-Benzyloxynapthyl-2-carboxaldehyde

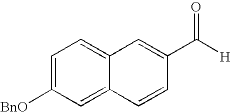

To a solution of 6-benzyloxynapth-2-ylmethyl alcohol (7.1 gm, 1 eq, 27.12 mmol) obtained in step 2 of preparation 4 and activated molecular sieves (4 A) in dry DCM (135 mL), PDC (11.2 gm, 1.1 eq, 29.83 mmol) was added at 0° C. After the addition, the reaction mixture was allowed to stir at RT for 15 hr. Reaction mixture was filtered through celite, filtrate was dried (Na₂SO₄), condensed, and the residue was chromatographed using ethyl acetate and hexane to obtain the title compound as white solid (4.15 gm, 59% yield). Mp: 102-104° C.

¹H NMR (CDCl₃, 200 MHz) δ: 5.18 (s, 2H); 7.22-7.47 (aromatics, 7H); 7.73-7.90 (aromatics, 3H); 8.22 (s, 1H); 10.06 (s, 1H).

IR (neat) cm⁻¹: 2924, 1694, 1617.

Mass m/z (CI): 263 [M+1].

### Preparation 5

### Methyl 3-(6-bezyloxynapth-2-yl)prop-2-enoate

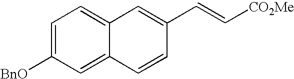

To a stirred solution of 60% NaH (915 mg, 1.5 eq, 22.90 mmol) in dry THF (60 mL) at 0° C., trimethylphosphonoacetate (3.7 mL, 1.5 eq, 22.90 mmol) in dry THF (5 mL) was added drop wise. After the addition reaction mixture was stirred at RT for 1 h. Then again at 0° C., 6-benzyloxynapthyl-2-carboxaldehyde (4.0 g, 1 eq, 15.27 mmol) obtained in step 3 of preparation 4, in dry THF (10 mL) was added drop wise and after the addition stirring was continued for 16 hr RT. Reaction mixture was concentrated to dryness, diluted with ethyl acetate (200 mL) and washed with water (2×150 mL). Organic layer was dried (Na₂SO₄), condensed, and the residue was chromatographed using ethyl acetate and hexane to obtain the title compound as white solid (4.6 g, 95% yield). Mp: 132-134° C.

¹H NMR (CDCl₃, 200 MHz) δ: 3.84 (s, 3H); 5.21 (s, 2H); 6.52 (d, J=16 Hz, 1H); 7.20-7.90 (aromatics, 11H).

IR (neat) cm⁻¹: 2925, 1718, and 1620.

Mass m/z(CI): 319 [M+1].

### Preparation 6

### 1,2,3,4-tetrahydro-2-(3-Methanesulfonyloxypropyl)-6-(methanesulfonyloxy)naphthalene

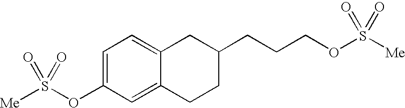

### Step 1: Methyl-3-(6-hydroxy-1,2,3,4-tetrahydronapth-2-yl)propionate

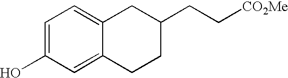

A solution of Methyl 3-(6-bezyloxynapth-2-yl)prop-2-enoate (4.6 g, 1 eq, 14.46 mmol) obtained in preparation 5 and 10% Pd—C (4.6 g) in ethyl acetate (250 mL) was kept in Parr hydrogenator at 60 psi H₂ pressure and at RT for 24 h. Reaction mixture was filtered through celite, dried (Na₂SO₄), condensed, and the residue was chromatographed using ethyl acetate and hexane to obtain the title compound as thick mass (3.26 g, 90% yield).

Mass m/z (ES): 252 [M+18], 257 [M+23].

### Step 2: 3-(6-Hydroxy-1,2,3,4-tetrahydronapth-2-yl)propan-1-ol

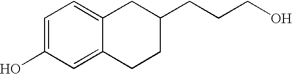

A solution of Methyl-3-(6-hydroxy-1,2,3,4-tetrahydronapth-2-yl)propionate (3.26 g, 1 eq, 14.17 mmol) obtained in step 1 of preparation 6, in dry THF (140 mL) was cooled up to −70° C., and then DIBAL (35.1 mL, 3 eq, 42.52 mmol) was added drop wise with constant stirring at −70° C. After the addition, the reaction mixture was slowly allowed to attain RT (5 h). Reaction mixture was quenched with Methanol (175 mL), followed by the addition of saturated solution of Na₂SO₄. Finally reaction mixture was filtered through celite. Filtrate was dried (Na₂SO₄), condensed, and the residue was chromatographed using ethyl acetate and hexane to obtain the title compound as thick mass (800 mg, 28% yield).

Mass m/z (CI): 207 [M+1].

### Step 3: 1,2,3,4-tetrahydro-2-(3-Methanesulfonyloxypropyl)-6-(methanesulfonyloxy)naphthalene

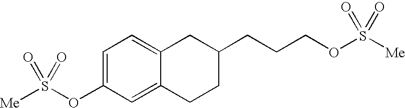

To a stirred solution of 3-(6-Hydroxy-1,2,3,4-tetrahydronapth-2-yl)propan-1-ol (720 mg, 1 eq, 1.36 mmol) obtained in step-2 of preparation 6, DMAP (catalytic amount) and Et₃N (3.9 mL, 6 eq, 28.41 mmol) in dry DCM (24 mL) at 0° C., methanesulfonylchloride (1.10 mL, 3 eq, 14.21 mmol) was added and stirring was continued for 5 h. The reaction mixture was diluted with 50 mL of DCM and washed with citric acid solution followed by water and brine. Organic layer was dried Na₂SO₄), condensed, and the residue was chromatographed using ethyl acetate and hexane to obtain the title compound as thick mass (800 mg, 47% yield).

¹H NMR (CDCl₃, 400 MHz) δ: 1.35-1.44 (m, 1H); 1.44-1.55 (m, 2H); 1.65-1.80 (m, 1H); 1.82-1.90 (m, 2H); 1.90-2.0 (m, 1H); 2.41 (dd, J=16.3, 10.6 Hz, 1H); 2.80-2.90 (m, 2H); 3.02 (s, 3H); 3.12 (s, 3H); 4.26 (t, J=6.8 Hz, 2H); 6.99-7.02 (aromatics, 2H); 7.02-7.10 (aromatics, 1H).

IR (neat) cm⁻¹: 2939, 1605, and 1496.

Mass m/z (CI): 363 [M+1].

### Preparation 7

### 3-(5-methanesulfonyloxyindol-1-yl)propyl bromide

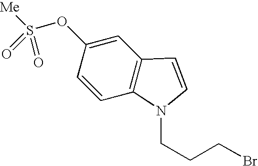

### Step 1: 5-(Methanesulfonyloxy)indole

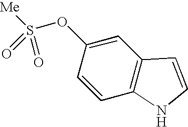

To a stirred solution of 5-hydroxyindole (5 g, 1 eq, 37.59 mmol), DMAP (catalytic amount) and Et₃N (10.5 mL, 2 eq, 75.19 mmol) in dry DCM (190 mL) at 0° C., methanesulfonylchloride (2.92 mL, 1 eq, 37.59 mmol) was added and stirring was continued for 5 hr. The reaction mixture was diluted with 50 mL of DCM and washed with Citric acid solution followed by water and brine. Organic layer was dried (Na₂SO₄), condensed, and the residue was chromatographed using ethyl acetate and hexane to obtain the title compound as brown color solid (5.5 g, 69% yield). Mp: 94-96° C.

¹H NMR (CDCl₃, 200 MHz) δ: 3.11 (s, 3H); 6.55 (s, 1H); 7.09 (dd, J=8.8 Hz, 2.4 Hz, 1H); 7.24-7.28 (aromatics, 1H); 7.36 (d, J=8.8 Hz, 1H); 7.54 (s, 1H); 8.31 (bs, NH).

IR (neat) cm⁻¹: 3397, 2924, 1479, and 1365.

Mass m/z (CI): 212 [M+1].

### Step 2: 3-(5-methanesulfonyloxyindol-1-yl)propyl bromide

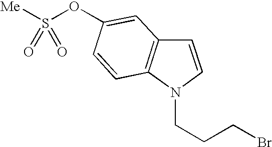

A mixture of (5-Methanesulfonyloxy)indole (5.5 g, 1 eq, 23.69 mmol) obtained in step 1 of preparation 7, and powdered KOH (1.99 g, 1.5 eq, 35.53 mmol) in dry DMSO (120 mL) was stirred at RT for 20 min. To that 1,3-Dibromopropane (7.2 mL, 3 eq, 71.07 mmol) was added drop wise and the stirring was continued for 1 h at RT. Reaction mixture was diluted with ethyl acetate (200 mL) and washed with water (2×100 mL). Organic layer was dried (Na₂SO₄), condensed, and the residue was chromatographed using ethyl acetate and hexane to obtain the title compound as thick mass (3.3 g, 42% yield).

¹H NMR (CDCl₃, 200 MHz) δ: 2.31 (quintet, J=6.2 Hz, 2H); 3.10 (s, 3H); 3.26 (t, J=6.2 Hz, 2H); 4.31 (t, J=6.2 Hz, 2H); 6.49 (d, J=2.4 Hz, 1H); 7.08-7.37 (aromatics, 3H); 7.50 (d, J=2.2 Hz, 1H).

IR (neat) cm⁻¹: 2932, 1481, and 1362.

Mass m/z (CI): 332 [M (⁷⁹Br)+1], 334 [M (⁸¹Br)+1].

### Preparation 8

### 3-(Indol-1-yl)propyl bromide

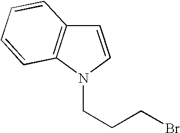

A mixture of indole (3 g, 1 eq, 25.63 mmol) and powdered KOH (2.18 g, 1.5 eq, 38.95 mmol) in dry DMSO (128 mL) was stirred at RT for 20 min. To that 1,3-dibromopropane (7.81 mL, 3 eq, 76.91 mmol) was added drop wise and stirring was continued for 1.5 h at RT. Reaction mixture was diluted with ethyl acetate (150 mL) and washed with water (2×100 mL). Organic layer was dried (Na₂SO₄), condensed, and the residue was chromatographed using ethyl acetate and hexane to obtain the title compound as thick mass (2.1 g, 35% yield).

¹H NMR (CDCl₃, 200 MHz) δ: 2.34 (quintet, J=6.2 Hz, 2H); 3.30 (t, J=6.2 Hz, 2H); 4.33 (t, J=6.2 Hz, 2H); 6.5 (d, J=2.8 Hz, 1H); 7.07-7.25 (aromatics, 3H); 7.37 (d, J=8 Hz, 1H); 7.63 (d, J=8 Hz, 1H).

IR (neat) cm⁻¹: 2932, 1463, and 1314.

Mass m/z(CI): 238 [M (⁷⁹Br)+1], 240 [M (⁸¹Br)+1].

### Preparation 9

### 3-(1,2,3,4-tetrahydroquinolin-1-yl)propyl bromide

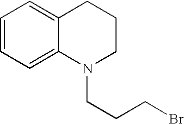

A mixture of 1, 2,3,4-tetrahydroquinoline (5 g, 1 eq, 37.59 mmol), 1,3-Dibromopropane (23 mL, 6 eq, 225.56 mmol) and anhydrous Na₂CO₃ (11.9 g, 3 eq, 112.77 mmol) in dry DMF (375 mL) was stirred at 70° C. for 4 hr. Reaction mixture was diluted with ethyl acetate (200 mL) and washed with water (2×100 mL). Organic layer was dried (Na₂SO₄), condensed, and the residue was chromatographed using ethyl acetate and hexane to obtain the title compound as thick mass (3.5 gm, 37% yield).

¹H NMR (CDCl₃, 200 MHz) δ: 1.95 (quintet, J=6.2 Hz, 2H); 2.15(quintet, J=6.6 Hz, 2H); 2.75 (t, J=6.2 Hz, 2H); 3.30 (t, J=5.5 Hz, 2H); 3.39-3.51 (m, 4H); 6.53-6.61(aromatics, 2H); 6.93-7.08 (m, 2H).

IR (neat) cm⁻¹: 3383(b), 2930, 2842, 1601, 1503

Mass m/z (CI): 254 [M (⁷⁹Br)+1], 256 [M (⁸¹Br)+1].

### Preparation 10

### 3-(2,3-dihydroindol-1-yl)propyl bromide

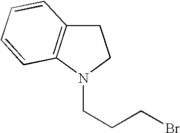

A mixture of indoline (3 g, 1 eq, 25.20 mmol), 1,3-di-bromopropane (15.4 mL, 6 eq, 151.26 mmol) and anhydrous Na₂CO₃ (8.0 g, 3 eq, 75.63 mmol) in dry DMF (250 mL) was stirred at 70° C. for 4 h. Reaction mixture was diluted with ethyl acetate (200 mL) and washed with water (2×100 mL). Organic layer was dried (Na₂SO₄), condensed, and the residue was chromatographed using ethyl acetate and hexane to obtain the title compound as thick mass (2.8 g, 47% yield).

¹H NMR (CDCl₃, 200 MHz) δ: 2.14 (quintet, J=6.2 Hz, 2H); 2.96 (t, J=8.1 Hz, 2H); 3.23 (t, J=6.4 Hz, 2H); 3.34 (t, J=8.1 Hz, 2H); 3.53 (t, J=6.2 Hz, 2H); 6.51 (d, J=8.1 Hz, 1H); 6.65 (t, J=7.2 Hz, 1H); 7.03-7.09 (aromatics, 2H).

IR (neat) cm⁻¹: 2925, 1606, and 1489.

Mass m/z(CI): 240 [M (⁷⁹Br)+1], 242 [M (⁸¹Br)+1].

### Preparation 11

### Ethyl 2-methyl-2-(3-phenoxy)propanoate

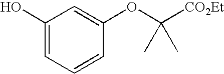

The title compound was prepared following a literature procedure described in (Ref: JMC, 2001, 44, 2061).

¹H NMR (CDCl₃, 200 MHz) δ: 1.25 (t, J=7.1 Hz, 3H); 1.60 (s, 6H); 4.24 (q, J=7.1 Hz, 2H); 5.35 (bs, 1H); 6.38-6.49 (aromatics 3H); 7.08 (t, J=7.8 Hz, 1H)

IR (neat) cm⁻¹: 3418, 2989, 2940, 1732, 1595, 1486.

Mass m/z (CI): 225 [M+1].

### Preparation 12

### 4-(Methanesulfonyloxy)phenol

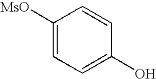

To a stirred solution of Quinol (5 g, 1 eq, 45.45 mmol), Et₃N (12.7 mL, 2 eq, 90.9 mmol) and DMAP (1.1 g, 0.2 eq, 9.09 mmol) in dry THF (955 mL) at 0° C., Mesyl chloride (2.6 mL, 0.75 eq, 34.09 mmol) was added drop wise. After the addition, stirring was continued at RT for 3 h. Reaction mixture was concentrated to dryness, diluted with ethyl acetate (400 mL) and washed with 10% citric acid solution (300 mL). Organic layer was dried Na₂SO₄), condensed, and the residue was chromatographed using ethyl acetate and hexane to obtain the title compound as white solid (3 g, 35% yield). Mp: 82-84° C.

¹H NMR (CDCl₃, 200 MHz) δ: 3.13 (s, 3H); 5.44 (bs, 1H); 6.83 (t, J=9.1 Hz, 1H); 7.15 (t, J=9.1 Hz, 1H).

IR (neat) cm⁻¹: 3455, 2989, 2940, 1599, 1505.

Mass m/z (CI): 189 [M+1].

### Preparation 13

### 3-(4-Methanesulfonyloxyphenoxy)propylbromide

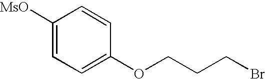

A mixture of 4-mesyloxy phenol (200 mg, 1 eq, 1.06 mmol) obtained in preparation 12, 1,3-Dibromo propane (0.54 mL, 5 eq, 5.3 mmol) and powdered anhydrous K₂CO₃ (439 mg, 3 eq, 3.18 mmol) in acetone (22 mL) was stirred at 60° C. for 18 h. Reaction mixture was concentrated to dryness, diluted with ethyl acetate (100 mL) and washed with water (2×75 mL). Organic layer was dried (Na₂SO₄), condensed, and the residue was chromatographed using ethyl acetate and hexane to obtain the title compound as thick mass (200 mg, 61% yield).

¹H NMR (CDCl₃, 200 MHz) δ: 2.32 (quintet, J=6.1 Hz, 2H); 3.11 (s, 3H); 3.60 (t, J=6.3 Hz, 2H); 4.10 (t J=5.8 Hz, 2H); 6.91 (t, J=9.1 Hz, 1H); 7.21 (t, J=9.1 Hz, 1H).

IR (neat) cm⁻¹: 3026, 2929, 1593, 1501.

Mass m/z (CI): 309 [M (⁷⁹Br)+1], 311 [M (⁸¹Br)+1].

### Preparation 14

### 3-(Methanesulfonyloxy)phenol

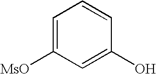

The title compound was prepared following the typical procedure described for preparation 12.

¹H NMR (CDCl₃, 200 MHz) δ: 3.15 (s, 3H); 6.79-6.86 (aromatics, 3H); 7.26 (t, J=9 Hz, 1H).

IR (neat) cm⁻¹: 3461, 3033, 2939, 1603, 1481.

Mass m/z (CI): 189 [M+1].

### Preparation 15

### 3-(3-Methanesulfonyloxyphenoxy)propylbromide

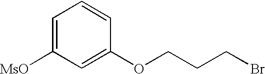

The title compound was prepared following the typical procedure described for preparation 13.

¹H NMR (CDCl₃, 200 MHz) δ: 2.32 (quintet, J=6.3 Hz, 2H); 3.14 (s, 3H); 3.60 (t, J=6.3 Hz, 2H); 4.11 (t, J=5.8 Hz, 2H); 6.80-6.90 (aromatics, 3H); 7.25-7.35 (aromatics, 1H).

IR (neat) cm⁻¹: 3028, 2938, 1607, 1586, 1485.

Mass m/z (CI): 309 [M(⁷⁹Br)+1], 311 [M(⁸¹Br)+1].

### Preparation 16

### Ethyl 2-methyl-2-[4-{3-(methanesulfonyloxy)propyl}phenoxy]propanoate

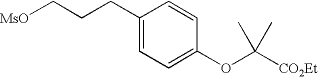

### Step 1: 3-(4-hydroxyphenyl)propan-1-ol

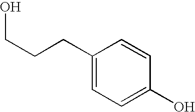

A suspension of LAH (10.5 g, w/w) in dry THF (500 mL) was refluxed for 3 hr. A solution of ethyl 3-(4-hydroxyphenyl)propionate (10 g, 1 eq, 55.55 mmol) in dry THF (50 mL) was added drop wise at reflux temperature. After the addition, reaction mixture was refluxed for 6 hr. Reaction mixture was quenched with ethyl acetate (40 mL, 4 eq with respect to LAH), followed by the addition of saturated Na₂SO₄ solution. To the workup mixture conc. HCl was added to adjust the pH at 7.0. Then reaction mixture was filtered through celite and washed with ethyl acetate. Combined filtrate was dried (Na₂SO₄), condensed, and the residue was chromatographed using ethyl acetate and hexane to obtain the title compound as white solid (5.7 g, 68% yield). Mp: 52-54° C.

¹H NMR (CDCl₃, 200 MHz) δ: 1.78-1.86 (m, 2H); 2.63 (t, J=7.9 Hz, 2H); 3.67 (t, J=6.3 Hz, 2H); 6.74(d, J=8.8 Hz, 2H); 7.05(d, J=8.8 Hz, 2H).

IR (neat) cm⁻¹: 3485, 3029, 2940, and 1505.

Mass m/z (CI): 152 [M+1].

### Step 2: Ethyl 2-methyl-2-[4-(3-hydroxypropyl)phenoxy]propionate

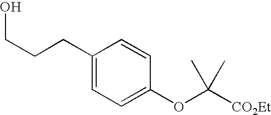

A mixture of 3-(4-hydroxyphenyl)propan-1-ol (3 g, 1 eq, 19.74 mmol), obtained in step 1 of preparation 16, ethyl 2-bromoisobutyrate (8.69 mL, 3 eq, 59.21 mmol), and powdered anhydrous K₂CO₃ (13.6 g, 5 eq, 98.7 mmol) in EtOH (98 mL) was heated at 70° C. for 17 h. Reaction mixture was condensed to dryness, diluted with ethyl acetate (200 mL) and washed with water (2×100 mL). Organic layer was dried (Na₂SO₄), condensed, and the residue was chromatographed using ethyl acetate and hexane to obtain the title compound as thick mass (4.7 g, 89% yield).

¹H NMR (CDCl₃, 200 MHz) δ: 1.25 (t, J=7.2 Hz, 3H); 1.57 (s, 6H); 1.82-1.89 (m, 2H); 2.64(t, J=7.2 Hz, 2H); 3.65(t, J=6.4 Hz, 2H); 4.23 (q, J=7.2 Hz, 2H); 6.77 (d, J=8.8 Hz, 2H); 7.05 (d, J=8.8 Hz, 2H)

IR (neat) cm⁻¹: 3406, 2939, 1733, and 1509.

Mass m/z (CI): 267 [M+1].

### Step 3: Ethyl 2-methyl-2-[4-(3-methanesulfonyloxypropyl)phenoxy]propionate

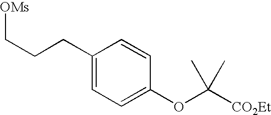

To a stirred solution of ethyl 2-methyl-2-[4-(3-hydroxypropyl)phenoxy]propionate (4.7 g, 1 eq, 17.66 mmol), obtained in step 2 of preparation 16, DMAP (catalytic amount) and Et₃N (4.9 mL, 2 eq, 35.34 mmol) in dry DCM (89 mL) at 0° C., methanesulfonylchloride (1.37 mL, 1 eq, 17.66 mmol) was added and stirring was continued for 5 h. The reaction mixture was diluted with 50 mL of DCM and washed with citric acid solution followed by water and brine. Organic layer was dried (Na₂SO₄), condensed, and the residue was chromatographed using ethyl acetate and hexane to obtain the title compound as thick mass (4 g, 66% yield).

¹H NMR (CDCl₃, 400 MHz) δ: 1.25 (t, J=7 Hz, 3H); 1.57 (s, 6H); 2.00-2.07 (m, 2H); 2.68 (t, J=7.2 Hz, 2H); 2.97 (s, 3H); 4.19-4.26 (m, 4H); 6.78 (d, J=8.8 Hz, 2H); 7.04 (d, J=8.8 Hz, 2H)

IR (neat) cm⁻¹: 2939, 1733, and 1509.

Mass m/z (ES): 345 [M+1], 362[M+18], 367[M+23].

### Preparation 17

### Ethyl 2-methyl-2-[4-(3-iodopropyl)phenoxy]propanoate

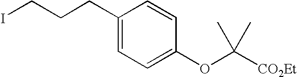

A mixture of Ethyl 2-methyl-2-[4-(3-methanesulfonyloxypropyl)phenoxy]propionate (500 mg, 1 eq, 1.45 mmol) obtained in preparation 16, and NaI (2.17 g, 10 eq, 14.5 mmol) in dry THF (8 mL) was stirred at 50° C. for 4 h. Reaction mixture was diluted with ethyl acetate (100 mL) and washed with water. Organic layer was dried (Na₂SO₄), condensed, and the residue was chromatographed using ethyl acetate and hexane to obtain the title compound as thick mass (495 mg, 90%).

Mass m/z (CI): 377 (M+1).

### Preparation 18

### Ethyl 2-methyl-2-[3-{3-(methanesulfonyloxy)propyl}phenoxy]propanoate

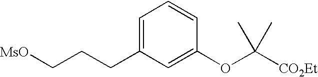

Prepared following the same procedure as described in the preparation 16.

¹H NMR (CDCl₃, 200 MHz) δ: 1.25 (t, J=7.1 Hz, 3H); 1.59 (s, 6H); 2.00-2.11 (m, 2H); 2.69 (t, J=7.5 Hz, 2H); 2.99 (s, 3H); 4.17-4.29 (m, 4H); 6.63-6.84 (aromatics 3H); 7.16 (t, J=7.8 Hz, 1H)

IR (neat) cm⁻¹: 2940, 1732.

Mass m/z (CI): 345 [M+1].

### Preparation 19

### ethyl 2-methyl-2-[3-(3-iodopropyl)phenoxy]propanoate

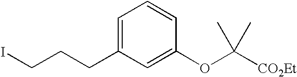

Prepared following the same procedure as described in the preparation 17 and using starting material obtained in Preparation 18.

¹H NMR (CDCl₃, 200 MHz) δ: 1.25 (t, J=7 Hz, 3H); 1.59 (s, 6H); 2.02-2.16 (m, 2H); 2.66 (t, J=7.4 Hz, 2H); 3.14 (t, J=7 Hz, 2H); 4.24 (q, J=7 Hz, 2H); 6.64-6.70 (aromatics, 2H); 6.82 (d, J=7.2 Hz, 1H); 7.14 (t, J=7.7 Hz, 1H)

IR (neat) cm⁻¹: 3381, 2985, 2935, 1733, 1584.

Mass m/z (CI): 377 [M+1].

### Preparation 20

### Ethyl-2-ethoxy-5-(4-aminophenyl)pentanoate

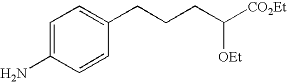

### Step 1: Ethyl 2-ethoxy-5-(4-nitrophenyl)penta-2,4-dienoate

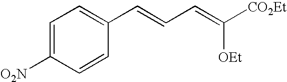

To a stirred solution of NaH (680 mg, 60% in oil, 1.5 eq, 16.95 mmol) in dry THF (50 mL) at 0° C., 2-ethoxy triethylphosphonoacetate (4.5 gm, 1.5 eq, 16.95 mmol) in dry THF (5 mL) was added drop wise. After the addition reaction mixture was stirred at RT for 2 h. Then again at 0° C., 4-Nitrocinnamaldehyde (2.0 g, 1 eq, 11.29 mmol), was added in portion wise and after the addition was over, stirring was continued for 6 h at RT. Reaction mixture was wuenched with methanol, concentrated to dryness, diluted with ethyl acetate (200 mL) and washed with water (2×150 mL). Organic layer was dried (Na₂SO₄), condensed, and the residue was chromatographed using ethyl acetate and hexane to obtain the title compound as a thick mass as a mixture of 2,3-E and Z isomers (TLC), 2.6 g, 80% yield). This was used for step 2 (next reaction).

### Step 2: Ethyl-2-ethoxy-5 (4-aminophenyl)pentanoate

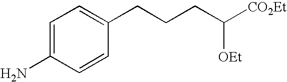

A solution of Ethyl 2-ethoxy-5-(4-nitrophenyl)penta-2,4-dienoate (2 g, 1 eq, 6.87 mmol) obtained in step 1 of preparation 20 and 10% Pd/C (2 g) in ethyl acetate (150 mL) was hydrogenated at 60 psi H₂ pressure and at RT for 7 h. Reaction mixture was filtered through celite, dried (Na₂SO₄), condensed, and the residue was chromatographed using ethyl acetate and hexane to obtain the title compound as thick mass (1.72 g, 94% yield).

¹H NMR (CDCl₃, 200 MHz) δ: 1.22 (t, J=7 Hz, 3H); 1.27 (t, J=7 Hz, 3H); 1.60-1.80 (m, 4H); 2.52 (t, J=6.8 Hz, 2H); 3.30-3.50 (m, 1H); 3.50-3.70 (m, 1H); 3.82 (d, J=5.3 Hz, 1H); 4.19 (q, J=7 Hz, 2H); 6.62 (d, J=8.3 Hz, 2H); 6.96 (d, J=8.3 Hz, 2H).

IR (neat) cm⁻¹: 3457, 2931, 1747, 1626, and 1517.

Mass m/z(CI): 265 [M], 266 [M+1].

### Preparation 21

### (S)-Ethyl 2-methoxy-3-(4-aminophenyl)propionate

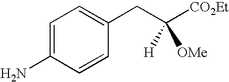

Step 1: To a solution of (S)-(4-nitrophenyl)glycine (10 g, 47.6 mmol) in a mixture of water (50 mL), H₂SO₄ (IM; 60 mL) and acetone (150 mL) at −5° C., was added under stirring, a solution of sodium nitrite (9.85 g, 142.8 mmol) in water (40 mL) drop wise over a period of 30 min. The reaction mixture was stirred at −5 to 0° C. for another 1.5 h, followed by stirring at room temperature for 16 h. Acetone was removed and then the reaction mixture was diluted with 500 mL ethyl acetate. Organic layer was washed with brine, dried over anhydrous Na₂SO₄, and concentrated. The crude mass was purified by crystallization from isopropyl acetate (9.0 g, 96%).

Mp: 134-136° C.

[α]_{D}: −25° (c 1.0, MeOH)

¹H NMR (CDCl₃) δ: 3.04 (dd, J=14, 7.8 Hz, 1H), 3.24 (dd, J=14, 4, Hz, 1H), 4.39 (dd, J=7.3, 4.1 Hz, 1H), 7.42 (d, J=8.7 Hz, 2H), 8.16 (d, J=8.7 Hz, 2H).

IR (neat) cm⁻¹: 3485, 3180, 2927, 1715, 1515, 1343.

Mass m/z (CI): 212 (M+1).

Step 2: (S)-2-Hydroxy-3-(4-nitrophenyl)propionic acid (9.0 g, 42.6 mmol), obtained from step (1) above, was dissolved in dry EtOH (300 mL). To this solution was added conc. H₂SO₄ (326 μL, 5.9 mmol), and refluxed for 5 to 6 h. The reaction mixture was neutralized with aqueous sodium bicarbonate. Ethanol was condensed on rotavapor, and the residue was dissolved in ethyl acetate. Organic layer was washed with aqueous sodium bicarbonate, water, brine, and then dried over anhydrous Na₂SO₄, and concentrated. Desired product was obtained from the crude mass by crystallizing from diisopropylether (8.0 g, 78.5%).

Mp: 74-76° C.

[α]_{D}: −13° (c 1.0, MeOH)

¹H NMR (CDCl₃) δ: 1.30 (t, J=7 Hz, 3H), 3.06 (dd, J=14, 7, Hz, 1H), 3.25 (dd, J=14, 4.3, Hz, 1H), 4.25 (q, J=7 Hz, 2H), 4.25 (dd, J=7, 4.3 Hz, 1H), 7.42 (d, J=8.7 Hz, 2H), 8.16 (d, J=8.7 Hz, 2H).

IR (neat) cm⁻¹: 3432, 2924, 1736, 1518, 1347.

Mass m/z (CI): 240 (M+1).

Step 3: To a mixture of (S)-Ethyl 2-Hydroxy-3-(4-nitrophenyl)propionate (12.5 g, 52.3 mmol), obtained in step (ii) of above, and powdered Ag₂O (36.3 g, 157 mmol) in dry acetonitrile (260 mL) was added methyl iodide (13 mL, 209.2 mmol) at room temperature. Activated molecular sieves (4 A) (12.5 g) were added and then the reaction mixture was stirred at room temperature for 16 h. The reaction mixture was filtered through celite, and concentrated. The crude mass was chromatographed using ethyl acetate and hexanes to obtain the desired product as viscous liquid (10.0 g, 75%).

[α]_{D}: −30.1° (c 1.0, MEOH)

¹H NMR (CDCl₃) δ: 1.24 (t, J=7.1 Hz, 3H); 3.09 (d, J=5.4 Hz, 1H); 3.12 (d, J=2.7 Hz, 1H); 3.35 (s, 3H); 3.96 (dd, J=7.5, 5.1 Hz, 1H); 4.19 (q, J=7.1 Hz, 2H); 7.39 (d, J=8.6 Hz, 2H); 8.13 (d, J=8.6 Hz, 2H).

IR (neat) cm⁻¹: 2995, 1747, 1604, 1521, 1343.

Mass m/z (CI): 254 (M+1).

Step 4: (S)-Ethyl 2-methoxy-3-(4-nitrophenyl)propionate (8.0, 31.6 mmol), obtained in step (3) above, was dissolved in dry methanol (200 mL). To this solution was added 10% Pd/C (2.5 g), and hydrogenated using hydrogen gas (20 psi) for 3-4 h. The reaction mixture was filtered through celite, and concentrated to a syrupy mass. After column chromatography using ethyl acetate/hexanes the desired product was isolated as thick liquid (7.0 g, quantitative).

[α]_{D}: −14.1° (c 1.0, MeOH).

Chiral HPLC: >98% ee.

¹H NMR (CDCl₃) δ: 1.23 (t, J=7.2 Hz, 3H), 2.91 (d, J=6.1 Hz, 2H), 3.30 (bs, 2H, NH₂), 3.34 (s, 3H), 3.88 (t, J=6.2 Hz, 1H), 4.17 (q, J=7.2 Hz, 2H), 6.62 (d, J=8.3 Hz, 2H), 7.01 (d, J=8.1 Hz, 2H).

IR (neat) cm⁻¹: 3372, 2985, 2932, 1739, 1627, 1519.

Mass m/z (CI): 223 (M), 234 (M+1), 192 (M-OMe).

### Preparation 22

### Ethyl 2-ethoxy-3-(4-aminophenyl)propionate

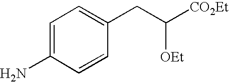

Step 1: Wittig salt from triethyl 2-ethoxyphosphonoacetate (26.5 g, 1.5 eq, 99.3 mmol) and NaH (50% in oil) (5.3 g, 2 eq, 132.4 mmol) was prepared in THF (350 mL) at 0° C. To this solid 4-nitrobenzaldehyde (10 g, 1 eq, 66.2 mmol) was added in portions at 0° C. and the resulting solution was stirred at RT for 16 h. The reaction mixture was diluted with ethyl acetate and washed with aqueous NH₄Cl. The crude contains ethyl p-nitro-2-ethoxycinnamate in both Z and E stereoisomers (11 g).

Step 2: Ethyl p-nitro-2-ethoxycinnamate obtained in step (1) was hydrogenated using 10% Pd—C—H₂ (60 psi) (11 g) in ethyl acetate (150 mL) at room temperature and chromatographed using ethyl acetate/hexane to yield the title compound as viscous oil (9.41 g, 60%).

¹H NMR (CDCl₃, 200 MHz): δ 1.16 (t, J=7.0 Hz, 3H), 1.22 (t, J=7.0 Hz, 3H), 2.90 (d, J=6.3 Hz, 2H), 3.30 (bs, 2H, NH₂), 3.35 (m, 1H), 3.55 (m, 1H), 3.94 (t, J=6.3 Hz, 1H), 4.15 (q, J=7.0 Hz, 2H), 6.62 (d, J=8.3 Hz, 2H), 7.03 (d, J=8.0 Hz, 2H).

IR (neat) cm⁻¹: 3372, 1738.

Mass m/z (CI): 238 (M+1), 192 (M-OC₂H₅).

### Preparation 23

### (S)-Methyl 3-ethoxy-4-(4-aminophenyl)butanoate

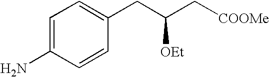

### Step 1: (S)-2-ethoxy-3-(4-nitrophenyl)propanoic acid

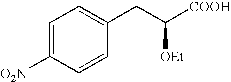

(S)-Ethyl 2-ethoxy-3-(4-nitrophenyl)propanoate (5 g, 1.0 eq, 18.72 mmol), prepared from L-4-nitro phenyl alanine was hydrolyzed by treating with LiOH.H₂O (1.18 g, 1.5 eq, 28.08 mmol) in MeOH-THF-water solvent mixture at RT for 3-4 h. The reaction mixture was condensed, diluted with water and acidified (pH at 3) with aq. HCl. Desired acid was extracted with ethyl acetate (200 mL). Organic layer was dried (Na₂SO₄), condensed, and the crude (3.66 g, 82% yield) was directly used for next reaction.

### Step 2: (S)-Methyl 3-ethoxy-4-(4-nitrophenyl)butanoate

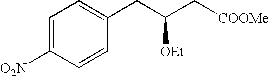

To a stirred solution of (S)-2-ethoxy-3-(4-nitrophenyl)propanoic acid (3.6 g, 1 eq, 15.10 mmol), obtained in step 1 of preparation 23, and Et₃N (2.1 mL, 1 eq, 15.10 mmol) in dry DCM (75 mL), isobutyl chloroformate (1.97 mL) was added at 0° C., and stirring was continued at RT for 1 h. Then at −5° C., CH₂N₂ (generated in 40 mL of diethyl ether) was added drop wise. After the addition, reaction was continued for 1 h at 0° C. Reaction mixture was diluted with DCM (50 mL), and washed with water. Organic layer was dried (Na₂SO₄), condensed, and dried under high vac. The crude mass thus obtained (3.9 g, 1 eq, 14.8 mmol) was dissolved in MeOH (80 mL) and Et₃N (6.2 mL, 3 eq, 44.4 mmol) was added. After the addition, Silver acetate (2.5 g, 1 eq, 14.8 mmol) was added at 0° C. in portions and stirring was continued for 1 h. Reaction mixture was condensed to dryness and the crude mass was chromatographed using ethyl acetate and hexane to obtain the title compound as thick mass. (2 g, 51% yield).

¹H NMR (CDCl₃, 400 MHz) δ: 1.07 (t, J=6.8 Hz, 3H); 2.42 (dd, J=15.6, 6.4 Hz, 1H); 2.56 (dd, J=15.6, 7 Hz, 1H); 2.87-2.98 (m, 2H); 3.33-3.41 (m, 1H); 3.47-3.55 (m, 1H); 3.69 (s, 3H); 3.96 (q, 1H); 7.4 (d, J=8.8 Hz, 2H); 8.15 (d, J=8.8 Hz, 2H).

IR (neat) cm⁻¹: 2976, 1738, 1603, and 1520.

Mass m/z(CI): 268 [M+1]

### Step 3: (S)-Methyl 3-ethoxy-4 (4-aminophenyl)butanoate

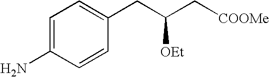

A solution of (S)-Methyl 3-ethoxy-4-(4-nitrophenyl)butanoate (2 g, 1 eq, 7.49 mmol) obtained in step 2 of preparation 23 and 10% Pd/C (500 mg) in ethyl acetate (250 mL) was hydrogenated at 40 psi H₂ pressure and at RT for 7 h. Reaction mixture was filtered through celite, dried (Na₂SO₄), condensed, and the residue was chromatographed using ethyl acetate and hexane to obtain the title compound as thick mass (1.3 g, 73% yield).

¹H NMR (CDCl₃, 400 MHz) δ:1.13 (t, J=7 Hz, 3H); 2.44 (d, J=6.2 Hz, 2H); 2.62 (dd, J=13.8, 7 Hz, 1H); 2.82 (dd) J=13.8, 5.8 Hz, 1H); 3.31-3.55 (m, 2H+NH); 3.65 (s, 3H); 3.85-3.94 (m, 1H); 6.62 (d, J=7.8 Hz, 2H); 7 (d, J=7.8 Hz, 2H).

IR (neat) cm⁻¹: 3370, 2975, 1736, 1626, and 1518.

Mass m/z(CI): 238 [M+1]

### Preparation 24

### 7-Methanesulfonyloxy-3,4-dihydro-2H-bezo[b][1,4]oxazine

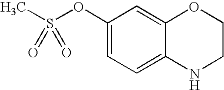

### Step 1: 3-Hydroxy-4-nitrophenol

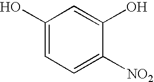

To a stirred solution of powdered KOH (10.6 g, 2 eq, 0.19 mol) in 60 mL of water, 5-flouro-2-nitrophenol (15 g, 1 eq, 0.095 mol) was added portion wise at 20-40° C. and the reaction mixture was heated at 90° C. for 28 h. Then every 4 h interval (3 times), 0.4 equiv. of powdered KOH was added to the reaction mixture and heating was continued for 15 h. Being guided by TLC (90% completion), reaction was stopped. Reaction mixture was diluted with 150 mL of water, acidified with 4N HCl and extracted with ethyl acetate (200 mL×2). Then organic layer was dried (Na₂SO₄), condensed, and the residue was chromatographed using ethyl acetate and hexane to obtain the title compound as yellow solid (9.9 g, 68% yield).

Mp: 106-108° C.

¹H NMR (CDCl₃, 400 MHz) δ: 5.97 (bs, —OH); 6.47 (dd, J=9.2, 2.4 Hz, 1H); 6.52 (d, J=2.4 Hz, 1H); 8.04 (d, J=9.2 Hz, 1H); 10.93 (s, —OH)

IR (KBr) cm⁻¹: 3362, 1622, 1533, 1291.

Mass m/z (CI): 156 [M+1]

### Step 2: 5-Methanesulfonyloxy-2-nitrophenol

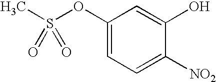

To a stirred solution of 3-hydroxy-4-nitrophenol (1 g, 1 eq, 6.45 mmol), obtained in step 1 of Preparation 24 and Et₃N (900 μL, 1 eq, 6.45 mmol) in dry DCM (130 mL) at 0° C., methanesulfonyl chloride (500 μL, 1 eq, 6.45 mmol) was added in a 15 min time and stirring was continued for another 15 min. The reaction mixture was diluted with 100 mL of DCM and washed with water (unreacted starting material went in aqueous layer). Organic layer was dried (Na₂SO₄), condensed, and the residue was chromatographed using ethyl acetate and hexane to obtain the title compound as yellow solid (570 mg, 38% yield).

Mp: 123-124° C.

¹H NMR (CDCl₃, 400 MHz) δ: 3.25 (s, 3H); 6.95 (dd, J=9.2, 2.8 Hz, 1H); 7.11 (d, J=2.8 Hz, 1H); 8.20 (d, J=9.2 Hz, 1H); 10.71 (s, —OH)

IR (KBr) cm⁻¹: 2943, 2600, 1696, 1669, 1629.

Mass m/z (CI): 234 (M+1]

### Step 3: 2-(5-methanesulfonyloxy-2-nitrophenoxy)ethyl bromide

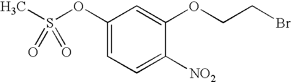

A mixture of 5-methanesulfonyloxy-2-nitrophenol (500 mg, 1 eq, 2.14 mmol) obtained in step 2 of Preparation 24, K₂CO₃ (890 mg, 3 eq, 6.43 mmol) and 1,2-dibromoethane (925 μL, 5 eq, 10.72 mmol) in 21 mL of dry acetone was stirred at 60° C. for 20 h. Being guided by TLC reaction was stopped. Acetone was removed, diluted with ethyl acetate (100 mL×2) and washed with water (100 mL). Organic layer was dried (Na₂SO₄), condensed, and the residue was chromatographed using ethyl acetate and hexane to obtain the title compound as yellow solid (420 mg, 58% yield).

Mp: 96-98° C.

¹H NMR (CDCl₃, 400 MHz) δ: 3.24 (s, 3H); 3.68 (t, J=6.4 Hz, 2H); 4.43 (t, J=6.4 Hz, 2H); 7.00 (dd, J=8.8, 2 Hz, 1H); 7.03 (d, J=2 Hz, 1H); 7.94 (d, J=8.8 Hz, 1H).

IR (neat) cm⁻¹: 3412, 2936, 1613, 1585, 1525.

Mass m/z (CI): 340 [M (⁷⁹Br)+1], 342 [M (⁸¹Br)+1]

### Step 4: 2-(5-Methanesulfonyloxy-2-aminophenoxy)ethyl bromide

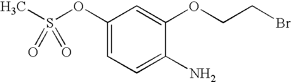

A solution of 2-(5-methanesulfonyloxy-2-nitrophenoxy)ethyl bromide (400 mg, 1 eq, 1.176 mmol) obtained in step 3 of Preparation 24 and 10% Pd/C (150 mg) in ethyl acetate (23 mL) was hydrogenated at H₂ balloon pressure and at 20-40° C. for 4 h. Reaction mixture was filtered through celite, dried (Na₂SO₄), condensed, and the residue was chromatographed using ethyl acetate and hexane to obtain the title compound as yellow solid (300 mg, 82% yield).

Mp: 69-70° C.

¹H NMR (CDCl₃, 400 MHz) δ: 3.09 (s, 3H); 3.67 (t, J=6 Hz, 2H); 4.32 (t, J=6 Hz, 2H); 6.67-6.75 (aromatics, 3H)

IR (KBr) cm⁻¹: 3437, 3327, 1616, 1511, 1345.

Mass m/z (CI): 310 [M (⁷⁹Br)+1], 312 [M (⁸¹Br)+1]

### Step 5: 7-Methanesulfonyloxy-3,4-dihydro-2H-bezo[b][1,4]oxazine

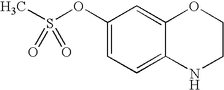

A mixture of 2-(5-methanesulfonyloxy-2-aminophenoxy)ethyl bromide (300 mg, 1 eq, 0.97 mmol) obtained in step 4 of Preparation 24 and K₂CO₃ (400 mg, 3 eq, 2.90 mmol) in 6 mL of dry DMF was stirred at 60° C. for 16 h. Being guided by TLC, reaction was stopped. Reaction mixture was diluted with ethyl acetate (50 mL) and washed with water (50 mL×2). Organic layer was dried (Na₂SO₄), condensed, and the residue was chromatographed using ethyl acetate and hexane to obtain the title compound as pale brown solid (190 mg, 85% yield).

Mp: 95-97° C.

¹H NMR (CDCl₃, 400 MHz) δ: 3.08 (s, 3H); 3.41 (t, J=4.4 Hz, 2H); 3.81 (bs, NH); 4.24 (t, J=4.4 Hz, 2H); 6.55 (d, J=8 Hz, 1H); 6.68-6.73 (aromatics, 2H)

IR (KBr) cm⁻: 3390, 2984, 1727, 1602, 1511.

Mass m/z (CI): 230 [M+1].

### Preparation 25

### 7-Methanesulfonyloxy-3,4-dihydro-2H-bezo[b][1,4]oxazin-3-one

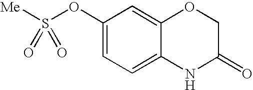

### Step 1: Ethyl 2-[2-nitro-5-methanesulfonyloxyphenoxy]acetate

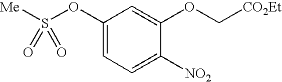

A mixture of 5-methanesulfonyloxy-2-nitrophenol (2.5 g, 10.73 mmol), obtained in step-2 of preparation 24, ethyl 2-bromoacetate (1.3 mL, 11.8 mmol), and anhydrous powdered K₂CO₃ in dry acetone (54 mL) was stirred at 20-40° C. for 16 h. Acetone was removed on rotavapor from the reaction mixture was diluted with ethyl acetate. Organic layer was washed with water, dried (Na₂SO₄), and condensed. The crude was used for next step.

Mass m/z (CI): 320 [M+1].

### Step 2: 7-Methanesulfonyloxy-3,4-dihydro-2H-bezo[b][1,4]oxazin-3-one

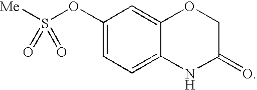

Ethyl 2-[2-nitro-5-methanesulfonyloxyphenoxy]acetate (3.7 g, crude), obtained in step 1 of preparation 25, was hydrogenolyzed using 10% Pd/C in ethyl acetate solvent (200 mL) at 20-40° C. over 10 psi H₂ pressure. Product was purified by column chromatography-(ethyl acetate/hexanes). Yield: 2.0 g (76%).

Mp: 201-202° C.

¹H NMR (DMSO-d₆, 400 MHz) δ: 3.34 (s, 3H); 4.61 (s, 2H); 6.90-7.00 (aromatics, 3H); 10.82 (s, 1H).

IR (KBr) cm⁻¹: 3440, 3087, 1687, 1509.

Mass m/z (CI): 244 [M+1].

### Preparation 26

### Ethyl 2-methyl-2-[4-(hydroxyl)phenoxy]butanoate

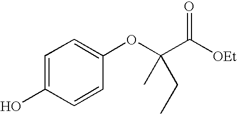

The said was prepared by hydrogenation of 2-(4-Benzyloxy-phenoxy)-2-methyl-butyric acid ethyl ester (1.2 gms, 3.7. mmol) in ethyl acetate with 10% Pd/C at RT for 5 hours.

¹H NMR (CDCl₃, 200 MHz) δ: 0.98 (t, J=7.3 Hz, 3H); 1.28 (t, J=6.94 Hz, 3H); 1.41 (s, 3H); 1.94 (q, J=7.1 Hz, 2H); 4.24 (q, J=7.1 Hz, 2H); 5.17(bs, 1H); 6.80-6.66 (m, 4H).

IR (neat) cm⁻¹: 3425, 2980, 2854, 1731, 1508.

Mass m/z (CI): 239 [M+1]

### Preparation 27

### Methyl-2-methyl-2-[4-(3-methanesulfonyloxypropyl)phenoxy]butanoate

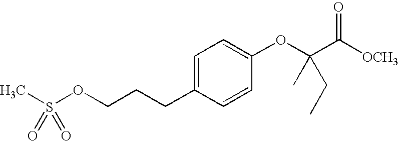

### Step 1: 2-methyl-2-[4-(3-hydroxypropyl)phenoxy]butanoic acid

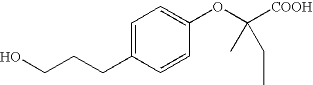

To a stirred solution of 3-(4-hydroxyphenyl)propan-1-ol (9 g, 1 eq, 59.2 mmol) obtained in step 1 of Preparation 16 in 296 mL of dry THF, powdered NaOH (21.6 g, 9 eq, 532.8 mmol) was added and was stirred at 2040° C. for 10 min. Then methyl ethyl ketone (52 mL, 10 eq, 592 mmol) was added at 20-40° C. and followed by stirring at 0° C. for 30 min. Then CHCl₃ (19 mL, 4 eq, 236.8 mmol) was added drop wise at 0° C. with vigorous stirring. After the addition of CHCl₃ reaction temperature was maintained at 0° C. for 2 h after which it was allowed to attain 20-40° C. while vigorous stirring for 24 h. Being guided by TLC, reaction was stopped. Reaction mixture was acidified with 4N HCl and extracted with ethyl acetate (200 mL×2). Organic layer was dried (Na₂SO₄), condensed, and the residue was chromatographed using ethyl acetate and hexane to obtain the title compound as thick mass (6.4 g, 43% yield). In this step compound was bit impure, and was characterized in the next step.

### Step 2: Methyl 2-methyl-2-[4-(3-hydroxypropyl)phenoxy]butanoate

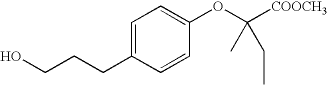

A solution of 2-methyl-2-[4-(3-hydroxypropyl)phenoxy]butanoic acid (2.0 g 1 eq, 7.93 mmol) obtained in step 1 of preparation 27 and conc. H₂SO₄ (86 μL, 0.2 eq, 1.59 mmol) in 40 mL of MeOH was heated at 70° C. (gentle reflux) for 17 h. Being guided by TLC, reaction was stopped. Reaction mixture was neutralized using solid NaHCO₃ and then MeOH was completely removed. Then it was diluted with ethyl acetate (200 mL) and washed with water (100 mL). Organic layer was dried (Na₂SO₄), condensed and the residue was chromatographed using ethyl acetate and hexane to obtain the title compound as thick mass (1.5 g, 71% yield).

¹H NMR (CDCl₃, 200 MHz) δ: 0.97 (t, J=7.4 Hz, 3H); 1.48 (s, 3H); 1.78-2.06 (m, 4H); 2.64 (t, J=7.7 Hz, 2H); 3.65 (t, J=6.4 Hz, 2H); 3.77 (s, 3H); 6.76 (d, J=8.4 Hz, 2H); 7.05 (d, J=8.4 Hz, 2H).

IR (neat) cm⁻¹: 3385, 2930, 1736, 1509.

Mass m/z (CI): 267 [M+1]

### Step 3: Methyl 2-methyl-2-[4-(3-methanesulfonyloxypropyl)phenoxy]butanoate

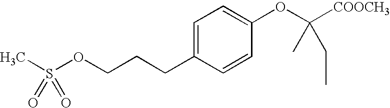

To a stirred solution of methyl 2-methyl-2-[4-(3-hydroxypropyl)phenoxy]butanoate (1.5 g, 1 eq, 5.63 mmol), obtained in step 2 of preparation 27, DMAP (138 mg, 0.2 eq, 1.12 mmol) and Et₃N (1.95 mL, 2.5 eq, 14.07 mmol) in dry DCM (28 mL) at 0° C., methanesulfonyl chloride (655 μL, 1.5 eq, 8.44 mmol) was added and stirring was continued for 3 h. The reaction mixture was diluted with 50 mL of DCM and washed with water. Organic layer was dried (Na₂SO₄), condensed, and the residue was chromatographed using ethyl acetate and hexane to obtain the title compound as thick mass (1.7 g, 88% yield).

¹H NMR (CDCl₃, 400 MHz) δ: 0.97 (t, J=7.4 Hz, 3H); 1.48 (s, 3H); 1.90-2.00 (m, 2H); 2.00-2.08 (m, 2H); 2.68 (t, J=7.6 Hz, 2H); 2.98 (s, 3H); 3.77 (s, 3H); 4.21 (t, J=6.4 Hz, 2H); 6.77 (d, J=8.4 Hz, 2H); 7.04 (d, J=8.4 Hz, 2H)

IR (neat) cm⁻¹: 2945, 1736, 1509.

Mass m/z (CI): 345 [M+1]

### Preparation 28

### Diastereomers of N1-[(αR)-2-hydroxy-1-phenylethyl]-2R/S)-2-[4-(3-hydroxypropyl)phenoxy]-2-methyl butamide

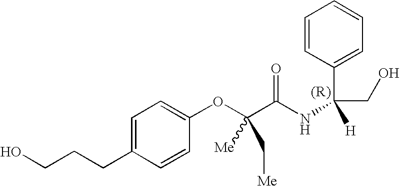

To a stirred solution of 2-methyl-2-[4-(3-hydroxypropyl)phenoxy]butanoic acid (5.8 g, 1 eq, 23.01 mmol) obtained in step 1 of Preparation 27, R-(−)-2-phenyl glycinol (9.5 g, 3 eq, 69.03 mmol) and DMAP (561 mg, 0.2 eq, 4.6 mmol) in 115 mL of dry DCM at 0° C., EDCI (6.2 g, 1.4 eq, 32.21 mmol) was added portion wise and stirring was continued at 0° C. for 30 min and it was allowed to stir at RT for 17 h. Being guided by TLC, reaction was stopped. Reaction mixture was diluted with 200 mL of CHCl₃ and washed with 10% Citric acid solution followed by NaHCO₃ solution. Organic layer was dried (Na₂SO₄), condensed, and the residue was chromatographed using silica gel and ethyl acetate/hexane to obtain the faster moving diastereomer (αR,2S which was eluted at 55% ethyl acetate/hexane, 2.4 g, thick mass) and the slower moving diastereomer (αR,2R which was eluted at 60% ethyl acetate/hexane, 2.2 g, thick mass). Stereochemistry (2S for faster moving diastereomer and 2R for slower moving diastereomer when used (R)-phenylglycinol) of these diastereomers was tentatively assigned. Total yield: 4.6 g (55%).

### Preparation 29

### N1-[(αR)-2-hydroxy-1-phenylethyl]-(2S)-2-[4-(3-hydroxypropyl)phenoxy]-2-methyl butamide

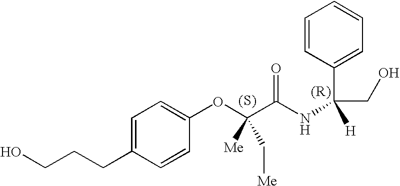

¹H NMR (CDCl₃, 400 MHz) δ: 1.05 (t, J=7.2 Hz, 3H); 1.38 (s, 3H); 1.80-1.92 (m, 3H); 1.98-2.05 (m, 1H); 2.66 (t, J=7.8 Hz, 2H); 3.66 (t, J=6.4 Hz, 2H); 3.90 (d, J=5.2 Hz, 2H); 5.13 (dt, J=7.2, 5.2 Hz, 1H); 6.84 (d, J=8.4 Hz, 2H); 7.07 (d, J=8.4 Hz, 2H); 7.25-7.38 (aromatics, 5H); 7.45 (d, J=7.2 Hz, NH)

IR (neat) cm⁻¹: 3413, 2933, 1658, 1506.

Mass m/z (CI): 372 [M+1]

[α]_{D}=−32° (c=1%, MeOH, 25° C.)

### Preparation 30

### N1-[(αR)-2-Hydroxy-1-phenylethyl]-(2R)-2-[4-(3-hydroxypropyl)phenoxy]-2-methyl butamide

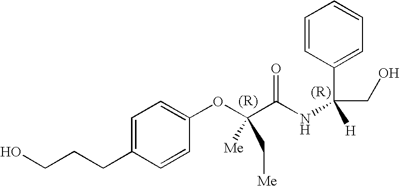

¹H NMR (CDCl₃, 400 MHz) δ: 0.93 (t, J=7.2 Hz, 3H); 1.43 (s, 3H); 1.70-1.90 (m, 3H); 1.90-2.00 (m, 1H); 2.67 (t, J=7.8 Hz, 2H); 3.67 (t, J=6.4 Hz, 2H); 3.90 (d, J=5.2 Hz, 2H); 5.12 (dt, J=7.2, 5.2 Hz, 1H); 6.90 (d, J=8.4 Hz, 2H); 7.11 (d, J=8.4 Hz, 2H); 7.27-7.37 (aromatics, 5H); 7.46 (d, J=7.2 Hz, NH).

IR (neat) cm⁻¹: 3410, 2932, 1656, 1507.

Mass m/z (CI): 372 [M+1]

[α]_{D}=+11.3 0 (c=1%, MeOH, 25° C.)

### Preparation 31

### (R)-(+)-Methyl-2-methyl-2-[4-(3-methanesulfonyloxypropyl)phenoxy]butanoate

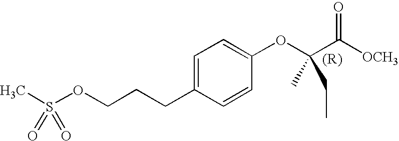

### Step 1: (R)-2-Methyl-2-[4-(3-hydroxypropyl)phenoxy]butanoic acid

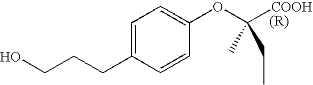

A solution of N1-[(αR)-2-hydroxy-1-phenylethyl](2R)-2-[4-(3-hydroxypropyl)phenoxy]-2-methyl butamide (1.64 g, 4.31 mmol) obtained in Preparation 30 in 35 mL of 6N HCl and 35 mL of Dioxane (1:1 mixture) was heated at 100° C. for 6 h. Being guided by TLC, reaction was stopped. Reaction mixture was diluted ethyl acetate (300 mL) and washed with water (200 mL). Organic layer was dried (Na₂SO₄), condensed and the residue, as a crude, was directly used for next reaction as this compound was pure enough to proceed for the next step. Crude yield (1.0 g, ˜95%)

### Step 2: Methyl (R)-2-Methyl-2-[4-(3-hydroxypropyl)phenoxy]butanoate

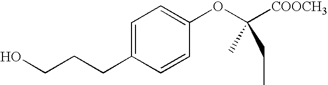

A solution of (R)-2-Methyl-2-[4-(3-hydroxypropyl)phenoxy]butanoic acid (1.0 g crude, 3.97 mmol) obtained in step 1 of Preparation 31 and conc. H₂SO₄ (52 μL, 0.2 eq, 0.79 mmol) in 24 mL of dry MeOH was heated at 70° C. (gentle reflux) for 17 h. Being guided by TLC, reaction was stopped. Reaction mixture was neutralized using solid NaHCO₃ and then MeOH was completely removed. Then it was diluted with ethyl acetate (200 mL) and washed with water (100 mL). Organic layer was dried (Na₂SO₄), condensed and the residue, as a crude, was directly used for next reaction as this compound was pure enough to proceed for the next step. Crude yield (0.95 g, 90%)

### Step 3: (+) Methyl (R)-2-methyl-2-[4-(3-methanesulfonyloxypropyl)phenoxy]butanoate

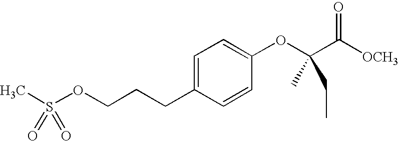

To a stirred solution of methyl (R)-2-methyl-2-[4-(3-hydroxypropyl)phenoxy]butanoate (950 mg, 1 eq, 3.57 mmol), obtained in step 2 of preparation 31, DMAP (87 mg, 0.2 eq, 0.714 mmol) and Et₃N (1.2 mL, 2.5 eq, 8.925 mmol) in dry DCM (18 mL) at 0° C., methanesulfonyl chloride (415 μL, 1.5 eq, 5.355 mmol) was added and stirring was continued for 3 h. The reaction mixture was diluted with 50 mL of DCM and washed with water. Organic layer was dried (Na₂SO₄), condensed, and the residue was chromatographed using ethyl acetate and hexane to obtain the title compound as thick mass (800 mg, 66% yield).

¹H NMR (CDCl₃, 400 MHz) δ: 0.97 (t, J=7.4 Hz, 3H); 1.48 (s, 3H); 1.90-2.00 (m, 2H); 2.00-2.08 (m, 2H); 2.68 (t, J=7.6 Hz, 2H); 2.98 (s, 3H); 3.77 (s, 3H); 4.21 (t, J=6.4 Hz, 2H); 6.77 (d, J=8.4 Hz, 2H); 7.04 (d, J=8.4 Hz, 2H).

IR (neat) cm⁻¹: 2945, 1736, 1509.

Mass m/z (CI): 345 [M+1].

[α]=+18° (c=1.1%, MeOH, 25° C.)

### Preparation 32

### (−)Methyl (S)-2-methyl-2-[4-(3-methanesulfonyloxypropyl)phenoxy]butanoate

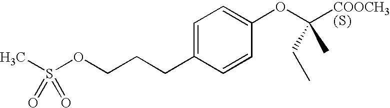

This compound was prepared using the faster moving diastereomer N1-[(αR)-2-hydroxy-1-phenylethyl]-(2S)-2-[4-(3-hydroxypropyl)phenoxy]-2-methylbutamide obtained in Preparation 29 and following the same procedure as described in Preparation 31.

[α]=−18° (c=1.25%, MeOH, 25° C.)

### Preparation 33

### 3-[4-(para-toluenesulfonyloxy)phenoxy]propylbromide

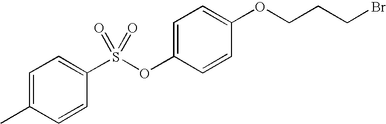

Step-1: 4-(para-Toluenesulfonyloxy)phenol

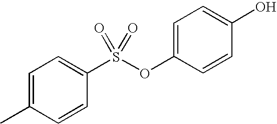

Obtained following the procedure for preparation 12 using p-toluenesulfonyl chloride instead of methanesulfonyl chloride.

Mp: 94-96° C.

Mass m/z (CI): 265 [M+1]

### Step 2: 3-[4-(para-Toluenesulfonyloxy)phenoxy]propylbromide

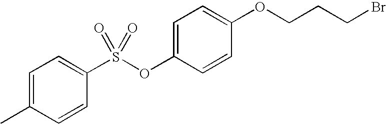

Obtained following the procedure for preparation 13 and using 4-(para-toluenesulfonyloxy)phenol as substrate.

Mp: 60-62° C.

¹H NMR (CDCl₃, 200 MHz) δ: 2.23-2.35 (m, 2H); 2.44 (s, 3H); 3.57 (t, J=6.3 Hz, 2H); 4.00 (t, J=5.8 Hz, 2H); 6.76 (d, J=9.3 Hz, 2H); 6.87 (d, J=9.3 Hz, 2H); 7.30 (d, J=8.2 Hz, 2H); 7.68 (d, J=8.2 Hz, 2H).

IR (neat) cm⁻¹: 2926, 1597, 1501, 1170.

Mass m/z (CI): 385 [M(⁷⁹Br)+1], 387 [M(⁸¹Br)+1].

### Preparation 34

### 5-(para-toluenesulfonyloxy)indole

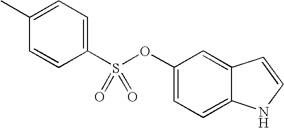

Title compound was prepared following the procedure for Step-1 of Preparation-7 and using para-toluenesulfonylchloride instead of methanesulfonyl chloride.

¹H NMR (CDCl₃, 200 MHz) δ: 2.44 (s, 3H); 6.77 (s, 1H); 6.80 (dd, J=9, 3 Hz, 1H); 7.10-7.30 (aromatics, 5H); 7.71 (d, J=8.1 Hz, 2H); 8.26 (bs, 1H).

IR (neat) cm⁻¹: 3421, 2925, 1176.

Mass m/z (CD): 287 [M+1].

### Preparation 35

### Ethyl 2-methyl-2-[4-(4-methanesulfonyloxybutyl)phenoxy]propanoate

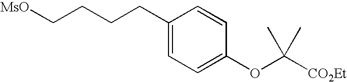

Obtained following the procedure for preparation 16 and starting from methyl 4-(4-hydroxyphenyl)butanoate. Spectral characterization for the intermediates and the title compound are given here.

### Step 1: 4-(4-Hydroxybutyl)phenol

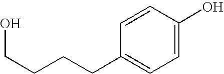

Mp: 56-58° C.

¹H NMR (CDCl₃, 200 MHz) δ: 1.22-1.70 (m, 4H); 2.52 (t, J=7 Hz, 2H); 3.40-3.80 (m, 2H and —OH); 6.72 (d, J=8.3 Hz, 2H); 6.97 (d, J=8.3 Hz, 2H).

IR (neat) cm⁻¹: 3361, 2937, 2859, 1613, 1515, 1239.

Mass m/z (ES): 184 [M+NH₄⁺], 189.3 [M+Na⁺], 350.1 [M₂+NH₄⁺], 355 [M₂+Na⁺].

### Step 2: Ethyl 2-methyl-2-[4-(4-hydroxybutyl)phenoxy]propanoate

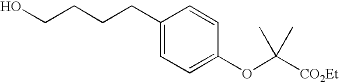

¹H NMR (CDCl₃, 200 MHz) δ: 1.25 (t, J=7.4 Hz, 3H); 1.57 (s, 6H); 1.65-1.82 (m, 4H); 2.56 (t, J=7.0 Hz, 2H); 3.64 (t, J=6 Hz, 2H); 4.23 (q, J=7.4 Hz, 2H); 6.76 (d, J=8.4 Hz, 2H); 7.00 (d, J=8.4 Hz, 2H).

IR (neat) cm⁻¹: 3375, 2938, 1734, 1509, 1142.

Mass m/z (CI): 281 [M+1].

### Step 3: Ethyl 2-methyl-2-[4-(4-methanesulfonyloxybutyl)phenoxy]propanoate

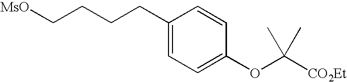

¹H NMR (CDCl₃, 200 MHz) δ: 1.25 (t, J=7.2 Hz, 3H); 1.57 (s, 6H); 1.65-1.80 (m, 4H); 2.59 (t, J=6.8 Hz, 2H); 2.97 (s, 3H); 4.15-4.30 (m, 4H); 6.76 (d, J=8.4 Hz, 2H); 7.00 (d, J=8.4 Hz, 2H).

IR (neat) cm⁻¹: 2940, 1732, 1509, 1175.

Mass m/z (CI): 359 [M+1].

### Preparation 36

### Ethyl 2-methyl-2-[3-(5-methanesulfonyloxypentyl)phenoxy]propanoate

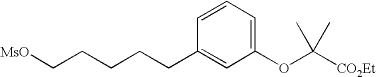

Obtained following the procedure for preparation 16 and starting from ethyl 3-(5-hydroxyphenyl)pentanoate.

Spectral characterization for the intermediates and the title compound are given here.

### Step 1: 3-(5-Hydroxypentyl)phenol

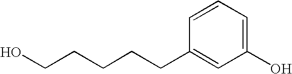

¹H NMR (CDCl₃, 400 MHz) δ: 1.35-1.42 (m, 2H); 1.55-1.65 (m, 4H); 1.78 (bs, OH); 2.58 (t, J=7.6 Hz, 2H); 3.64 (t, J=6.5 Hz, 2H); 5.63 (bs, OH); 6.63-6.66 (aromatics, 2H); 6.72 (d, J=7.5 Hz, 1H); 7.12 (dd, J=8.8, 7.5 Hz, 1H).

IR (neat) cm⁻¹: 3332, 2935, 1589, 1457.

Mass m/z(ES): 181 [M+1]

### Step 2: Ethyl 2-methyl-2-[3-(5-hydroxypentyl)phenoxy]propanoate

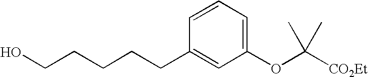

¹H NMR (CDCl₃, 400 MHz) δ: 1.24 (t, J=7.3 Hz, 3H); 1.30-1.42 (m, 2H); 1.58 (s, 6H); 1.54-1.65 (m, 4H+OH); 2.56 (t, J=7.6 Hz, 2H); 3.62 (t, J=6.4 Hz, 2H); 4.23 (q, J=7.3 Hz, 2H); 6.64 (dd, J=8.2, 2.0 Hz, 1H); 6.68 (t, J=1.9 Hz, 1H); 6.80 (d, J=7.5 Hz, 1H); 7.12 (t, J=7.8 Hz, 1H).

IR (neat) cm⁻¹: 3048, 2934, 1733, 1548, 1139.

Mass m/z(ES): 295 [M+1]

### Step 3: Ethyl 2-methyl-2-[3-(5-methanesulfonyloxypentyl)phenoxy]propanoate

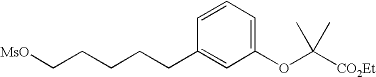

¹H NMR (CDCl₃, 400 MHz) δ: 1.24 (t, J=7.1 Hz, 3H); 1.38-1.48 (m, 2H); 1.58 (s, 6H); 1.60-1.70 (m, 2H); 1.70-1.80 (m, 2H); 2.56 (t, J=7.6 Hz, 2H); 2.98 (s, 3H); 4.18-4.25 (m, 4H); 6.65 (dd, J=8.0, 0.5 Hz, 1H); 6.68 (t, J=1.8 Hz, 1H); 6.79 (d, J=7.5 Hz, 1H); 7.12 (t, J=7.8 Hz, 1H).

IR (neat) cm⁻¹: 2939, 1733, 1502, 1176.

Mass m/z(ES): 373 [M+1]

### Preparation 37

### Ethyl 2-[3-(3-methanesulfonyloxypropyl)phenoxy]propanoate

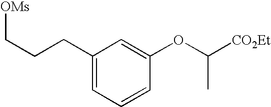

The title compound has been synthesized starting from 3-(3-hydroxypropyl)phenol, using ethyl 2-bromopropionate and following the procedure for preparation 16. Spectral data for the intermediates and the title compound are given here.

### Step 1: Ethyl 2-[3-(3-hydroxypropyl)phenoxy]propanoate

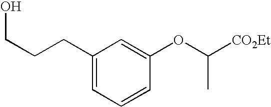

Yield: 84%

¹H NMR (CDCl₃, 400 MHz) δ: 1.25 (t, J=7.2 Hz, 3H); 1.61 (d, J=6.7 Hz, 3H); 1.78-1.94 (m, 2H); 2.67 (t, J=7.5 Hz, 2H); 3.65 (t, J=6.3 Hz, 2H); 4.21 (q, J=7.2 Hz, 2H); 4.73 (q, J=6.7 Hz, 1H); 6.65-9-6.85 (aromatics, 3H); 7.17 (d, J=7.8 Hz, 1H).

IR (neat) cm⁻¹: 3406, 2939, 1736.

Mass m/z (CI): 253 [M+1].

### Step 2: Ethyl 2-[3-(3-methanesulfonyloxypropyl)phenoxy]propanoate

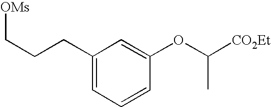

Yield: 85%

¹H NMR (CDCl₃, 400 MHz) δ: 1.25 (t, J=7.2 Hz, 3H); 1.61 (d, J=6.7 Hz, 3H); 2.00-2.20 (m, 2H); 2.59 (t, J=7.4 Hz, 2H); 3.00 (s, 3H); 4.15-4.30 (m, 4H); 4.73 (q, J=6.7 Hz, 1H); 6.68-9-6.85 (aromatics, 3H); 7.19 (d, J=7.8 Hz, 1H).

IR (neat) cm⁻¹: 2939, 1747, 1172.

Mass m/z (CI): 331 [M+1].

### Preparation 38

### 1-[4-(3-Methanesulfonyloxypropyl)phenoxy]cyclohexane-1-carboxylic acid, methyl ester

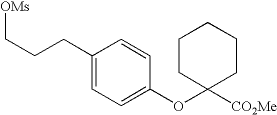

The title compound has been synthesized starting from 4-(3-hydroxypropyl)phenol, using cyclohexanone and following the procedure for Methyl 2-methyl-2-[4-(3-methanesulfonyloxypropyl)phenoxy]butanoate, preparation 27. Spectral data for the intermediates and the title compound are given here.

### Step 1: 1-[4-(3-Hydroxypropyl)phenoxy]cyclohexane-1-carboxylic acid, methyl ester

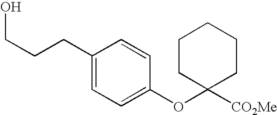

Yield: 38% (two step)

¹H NMR (CDCl₃, 400 MHz) δ: 1.48-1.72 (m, 6H); 1.82-1.92 (m, 4H); 2.04-2.18 (m, 2H); 2.63 (t, J=7.6 Hz, 2H); 3.65 (bs, —OH); 3.70 (t, J=8.3 Hz, 2H); 3.75 (s, 3H); 6.73 (d, J=8.6 Hz, 2H); 7.05 (d, J=8.6 Hz, 2H).

IR (neat) cm⁻¹: 3383, 2938, 2860, 1733, 1508, 1226, 1063.

Mass m/z (CI): 292 [M], 293 [M+1].

### Step 2: 1-[4-(3-Methanesulfonyloxypropyl)phenoxy]cyclohexane-1-carboxylic acid, methyl ester

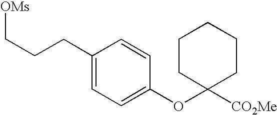

Yield: 79%

¹H NMR (CDCl₃, 400 MHz) δ: 1.50-1.77 (m, 6H); 1.82-1.92 (m, 2H); 2.00-2.08 (m, 4H); 2.08-2.14 (m, 2H); 2.67 (t, J=7.4 Hz, 2H); 2.98 (s, 3H); 3.76 (s, 3H); 4.21 (t, J=6.4 Hz, 2H); 6.74 (d, J=8.6 Hz, 2H); 7.04 (d, J=8.6 Hz, 2H).

IR (neat) cm⁻¹: 2937, 2859, 1733, 1508, 1353, 1226, 1174.

Mass m/z (CI): 388.3 [M+NH₄⁺], 758.5 [M₂+NH₄⁺].

### Preparation 39

### 1-[4-(3-Methanesulfonyloxypropyl)phenoxy]cyclopentane-1-carboxylic acid, methyl ester

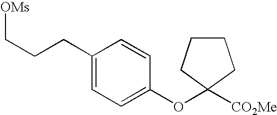

The title compound has been synthesized starting from 4-(3-hydroxypropyl)phenol, using cyclopentanone and following the procedure for methyl 2-methyl-2-[4-(3-methanesulfonyloxypropyl)phenoxy]butanoate, preparation 27. Spectral data for the intermediates and the title compound are given here.

### Step 1: 1-[4-(3-Hydroxypropyl)phenoxy]cyclopentane-1-carboxylic acid, methyl ester

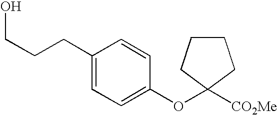

Yield: 60% (two step)

¹H NMR (CDCl₃, 400 MHz) δ: 1.70-1.90 (m, 6H); 2.10-2.30 m, 4H); 2.67 (t, J=7.6 Hz, 2H); 3.73 (s, 3H); 3.65(t, J=6.4); 6.66 (d, J=8.6 Hz, 2H); 7.04(d, J=8.6 Hz, 2H).

IR (neat) cm⁻¹: 3387, 2950, 2873, 1734, 1510, 1235, 1178.

Mass m/z (CI): 279 [M+11].

### Step 2: 1-[4-(3-Methanesulfonyloxypropyl)phenoxy]cyclopentane-1-carboxylic acid, methyl ester

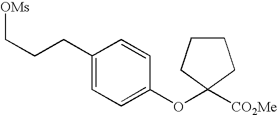

Yield: 54%

¹H NMR (CDCl₃, 400 MHz) δ: 1.70-1.86 (m, 4H); 2.00-2.08 (m, 2H); 2.12-2.21 m, 2H); 2.21-2.30 (m, 2H); 2.67 (t, J=7.6 Hz, 2H); 2.98 (s, 3H); 3.73 (s, 3H); 4.21 (t, J=6.2 Hz, 2H); 6.67 (d, J=8.6 Hz, 2H); 7.03 (d, J=8.6 Hz, 2H).

IR (neat) cm⁻¹: 2954, 2874, 1735, 1510, 1359, 1236, 1174.

Mass m/z (CI): 357 [M+1].

### Preparation 40

### 1-[4-(4-Methanesulfonyloxybutyl)phenoxy]cyclopentane-1-carboxylic acid, methyl ester

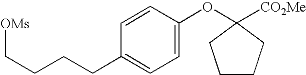

The title compound has been synthesized starting from 4-(4-hydroxybutyl)phenol, using cyclopentanone and following the procedure for methyl 2-methyl-2-[4-(3-methanesulfonyloxypropyl)phenoxy]butanoate, preparation 27. Spectral data for the intermediates and the title compound are given here.

### Step 1: 1-[4-(4-Hydroxybutyl)phenoxy]cyclopentane-1-carboxylic acid

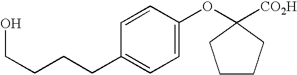

Yield: 59%

¹H NMR (CDCl₃, 200 MHz) δ: 1.55-1.70 (m, 4H); 1.70-1.90 (m, 4H); 2.15-2.22 (m, 2H); 2.22-35 (m, 2H); 2.56 (t, J=7.3 Hz, 2H); 3.64 (t, J=6.2 Hz, 2H); 4.50 (bs, —OH); 6.73 (d, J=9.0 Hz, 2H); 7.04 (d, J=9.0 Hz, 2H).

IR (neat) cm⁻¹: 3446, 2930, 2856, 1723, 1508, 1195.

Mass m/z (CI): 278 [M⁺], 279 [M+1].

### Step 2: 1-[4-(4-Hydroxybutyl)phenoxy]cyclopentane-1-carboxylic acid, methyl ester

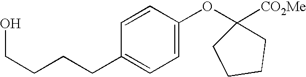

Yield: 84%

¹H NMR (CDCl₃, 400 MHz) δ: 1.55-1.70 (m, 4H+OH); 1.70-1.90 (m, 4H); 2.12-2.30 (m, 4H); 2.56 (t, J=7.4 Hz, 2H); 3.68 (t, J=6.2 Hz, 2H); 3.73 (s, 3H); 6.66 (d, J=8.8 Hz, 2H); 7.03 (d, J=8.8 Hz, 2H).

IR (neat) cm⁻¹: 3382, 2939, 1734, 1610, 1508, 1173.

Mass m/z (ES): 293 [M+1], 310.1 [M+NH₄⁺], 315 [M+Na⁺], 602.3 [M₂+NH₄⁺], 607.3 [M₂+Na⁺].

### Step 3: 1-[4-(4-Methanesulfonyloxybutyl)phenoxy]cyclopentane-1-carboxylic acid, methyl ester

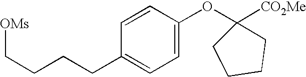

Yield: 72%

¹H NMR (CDCl₃, 400 MHz) δ: 1.67-1.88 (m, 8H); 2.10-2.20 (m, 2H); 2.20-2.30 (m, 2H); 2.58 (t, J=7.3 Hz, 2H); 2.98 (s, 3H); 3.73 (s, 3H); 4.22 (t, J=6.3 Hz, 2H); 6.66 (d, J=8.6 Hz, 2H); 7.02 (d, J=8.6 Hz, 2H).

IR (neat) cm⁻¹: 2945, 1735, 1608, 1509, 1173.

Mass m/z (CI): 370 [M⁺].

### Preparation 41

### 1-[4-(3-Iodopropyl)phenoxy]cyclopentane-1-carboxylic acid, methyl ester

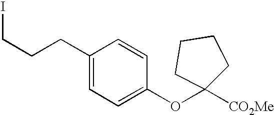

The title compound was prepared using the procedure used for preparation 17 and using 1-[4-(3-methanesulfonyloxypropyl)phenoxy]cyclopentane-1-carboxylic acid, methyl ester, obtained in preparation 39.

Mass m/z (CI): 389 [M+1].

### EXAMPLE 1

### (S)-Ethyl 2-methoxy-3-[4-(6-methanesulfonyloxynapth-2-ylmethylamino)phenyl]propanoate

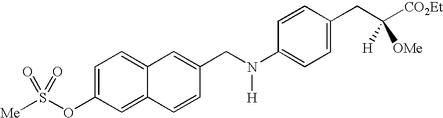

A mixture of 6-methanesulfonyloxynapthyl-2-carboxaldehyde (500 mg, 1 eq, 2 mmol) obtained in preparation 1, S ethyl 2-methoxy-3-(4-aminophenyl)propionate (446 mg, 1 eq, 2 mmol), (obtained in preparation 21), activated molecular sieves (4 A), and p-TsOH (38 mg, 0.1 eq, 0.2 mmol) in dry DCM (5 mL) were stirred at RT for 16 h. The reaction mixture was diluted with ethyl acetate (100 ml), washed with aq. sodium bicarbonate, dried (Na₂SO₄), condensed (rotavapor), and dried under high vac. The crude mass (825 mg) was dissolved in dry methanol (10 ml) and conc HCl (181 μL) was added at 0° C., followed by NaB(CN)H₃ (172 mg, 1.5 eq, 2.727 mmol) in portions. The reaction mixture was stirred at 0° C. for 3 h, after that it was diluted with ethyl acetate (100 mL). The organic layer was washed with aq. sodium bicarbonate, dried (Na₂SO₄), and condensed. The residue was chromatographed using ethyl acetate and hexanes to obtain the title compound as white solid (560 mg, 68% yield). Mp: 94-96° C.

¹H NMR (CDCl₃, 400 MHz) δ: 1.21 (t, J=7.0 Hz, 3H); 2.8-2.9 (m, 2H); 3.18 (s, 3H); 3.34 (s, 3H); 3.88 (dd, J=7.3, 6 Hz, 1H); 4.16 (q, J=7.0 Hz, 2H); 4.49 (s, 2H); 6.58 (d, J=8.3 Hz, 2H); 7.03 (d, J=8.3 Hz, 2H); 7.39 (dd, J=8.8, 2.4 Hz, 1H); 7.54 (dd, J=8.3, 1.4 Hz, 1H); 7.74 (d, J=2 Hz, 1H); 7.81-7.85 (aromatics, 3H).

IR (neat) cm⁻¹: 3380, 2927, 1727, 1614, and 1522.

Mass m/z(CI): 458 [M+1].

The following examples (examples 2-4) were made using the typical procedure described for example 1.

### EXAMPLE 2

### Ethyl 2-ethoxy-3-[4-{6-methanesulfonyloxynapth-2-ylmethylamino}phenyl]propanoate

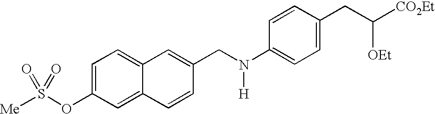

White solid, Mp: 118-120° C., Yield: 520 mg, 52%.

¹H NMR (CDCl₃, 200 MHz) δ: 1.11-1.24 (m, 6H); 2.87 (d, J=6.7 Hz, 2H); 3.16 (s, 3H); 3.22-3.42 (m, 1H); 3.48-3.68 (m, 1H); 3.92 (t, J=6.7 Hz, 1H); 4.13 (q, J=7.0 Hz, 2H); 4.47 (s, 2H); 6.56 (d, J=8.3 Hz, 2H); 7.02 (d, J=8.3 Hz, 2H); 7.37 (dd, J=8.8, 2.4 Hz, 1H); 7.52 (d, J=8.8 Hz, 1H); 7.72 (d, J=2 Hz, 1H); 7.78-7.84 (aromatics, 3H).

IR (neat) cm⁻¹: 3381, 2928, 1731, 1614, and 1522.

Mass m/z(CI): 471 [M], 472 [M+1].

### EXAMPLE 3

### Ethyl 2-ethoxy-5-[4-{6-methanesulfonyloxynapth-2-ylmethylamino}phenyl]pentanoate

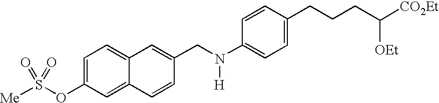

Yield: 580 mg, 72%.

¹H NMR (CDCl₃, 400 MHz) δ: 1.20 (t, J=7.4 Hz, 3H); 1.26 (t, J=7.3 Hz, 3H); 1.60-1.80 (m, 4H); 2.51 (t, J=7.3 Hz, 2H); 3.18 (s, 3H); 3.23-3.40 (m, 1H); 3.58-3.62 (m, 1H); 3.80 (t, J=6.8 Hz, 1H); 4.15-4.21 (m, 2H); 4.49 (s, 2H); 6.59 (d, J=8.8 Hz, 2H); 6.97 (d, J=8.8 Hz, 2H); 7.39 (dd, J=8.8, 2.4 Hz, 1H); 7.55 (dd, J=8.3, 1.5 Hz, 1H); 7.74 (d, J=2.4 Hz, 1H); 7.81-7.86 (aromatics, 3H).

IR (neat) cm⁻¹: 3404, 2931, 1740, 1614, and 1521.

Mass m/z (CI): 499 [M], 1,500 [M+1].

### EXAMPLE 4

### Ethyl 2-methyl-2-[4-{6-methanesulfonyloxynapth-2-ylmethylamino}phenoxy]propanoate

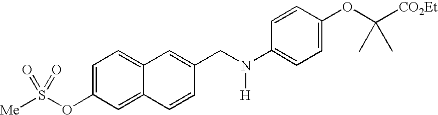

White solid, Mp: 116-118° C., Yield: 800 mg, 73%.

¹H NMR (CDCl₃, 200 MHz) δ: 1.27 (t, J=7 Hz, 3H); 1.50 (s, 6H); 3.18 (s, 3H); 4.00 (bs, NH); 4.22 (q, J=7 Hz, 2H); 4.45 (s, 2H); 6.54 (d, J=8.8 Hz, 2H); 6.77 (d, J=8.8 Hz, 2H); 7.34 (dd, J=8.8, 2.4 Hz, 1H); 7.54 (d, J=9.6 Hz, 1H); 7.74 (d, J=2.4 Hz, 1H); 7.80-7.87 (aromatics, 3H).

IR (neat) cm⁻¹: 3409, 2987, 2936, 1731, and 1512.

Mass m/z (CI): 458 [M+1].

### EXAMPLE 5

### Ethyl 2-ethoxy-3-[4-{3-(indol-1-yl)propyl amino}phenyl]propanoate

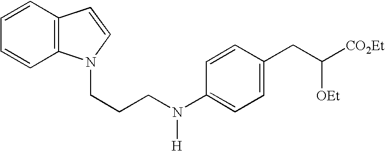

A mixture of Ethyl 2-ethoxy-3-(4-aminophenyl)propanoate (450 mg, 1 eq, 1.90 mmol) (obtained in preparation 22), 3-(indol-1-yl)propyl bromide (500 mg, 1.1 eq, 2.10 mmol) obtained in preparation 8, anhydrous K₂CO₃ (786 mg, 3 eq, 5.70 mmol), and TBAB (122 mg, 0.2 eq, 0.38 mmol) in dry toluene (13 mL) was stirred at 90° C. for 5 h. Reaction mixture was diluted with ethyl acetate (100 mL) and washed with water (2×100 mL). Organic layer was dried (Na₂SO₄), condensed, and the residue was chromatographed using ethyl acetate and hexane to obtain the title compound as thick mass (335 mg, 40% yield).

¹H NM (CDCl₃, 400 MHz) δ: 1.16 (t, J=7.3 Hz, 3H); 1.22 (t, J=7 Hz, 3H); 2.13 (quintet, J=6.8 Hz, 2H); 2.89 (d, J=6.3 Hz, 2H); 3.09 (t, J=7 Hz, 2H); 3.30-3.40 (m, 1H); 3.55-3.62 (m, 1H); 3.94 (t, J=6.3 Hz, 1H); 4.16 (q, J=7 Hz, 2H); 4.26 (t, J=6.3, 2H); 6.47 (d, J=8.8 Hz, 2H); 6.49 (dd, J=10, 4 Hz, 1H); 7.03 (d, J=8.3 Hz, 2H); 7.08-7.12 (aromatics, 2H); 7.20 (dt, J=8.3, 1.5 Hz, 1H); 7.34 (d, J=8.3 Hz, 1H); 7.64 (d, J=7.8 Hz, 1H).

IR (neat) cm⁻¹: 3393, 2928, 1739, 1616, and 1521.

Mass m/z(CI): 395 [M+1]. The following examples (examples 6-14) were made using the typical procedure described for example 5.

### EXAMPLE 6

### (S)-Methyl 2-methoxy-3-[4-{3-indol-1-yl)propylamino}phenyl]propanoate

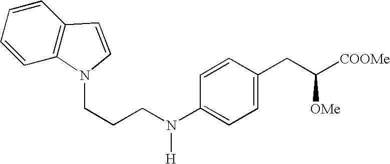

Yield: 400 mg, 52%

¹H NMR (CDCl₃, 200 MHz) δ: 2.14 (quintet, J=6.8 Hz, 2H); 2.91 (d, J=5.9 Hz, 2H); 3.09 (t, J=6.7 Hz, 2H); 3.35 (s, 3H); 3.72 (s, 3H); 3.91 (t, J=5.9 Hz, 1H); 4.27 (t, J=6.7, 2H); 6.45-6.55 (aromatics, 3H); 6.95-7.40 (aromatics, 6H); 7.65 (d, J=7.8 Hz, 1H).

IR (neat) cm⁻¹: 3394, 2926, 1743, 1614, and 1521.

Mass m/z (CI): 367 [M+1].

### EXAMPLE 7

### (S)-Ethyl-2-ethoxy-3-[4-{3-(5-methanesulfonyloxyindol-1-yl)propylamino}phenyl]propanoate

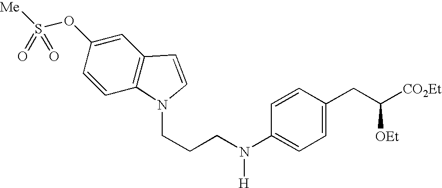

Yield: 600 mg, 65%.

¹H NMR (CDCl₃, 400 MHz) δ: 1.17 (t, J=7 Hz, 3H); 1.23 (t, J=7.3 Hz, 3H); 2.13 (quintet, J=6.9 Hz, 2H); 2.90 (d, J=6.8 Hz, 2H); 3.08 (t, J=6.8 Hz, 2H); 3.12 (s, 3H); 3.32-3.40 (m, 1H); 3.54-3.62 (m, 1H); 3.94 (d, J=6.8 Hz, 1H); 4.16 (q, 1=7 Hz, 2H); 4.26 (t, J=7 Hz, 2H); 6.47 (d, J=8.8 Hz, 2H); 6.52 (d, J=2.5 Hz, 1H); 7.03 (d, J=8.3 Hz, 2H); 7.12 (dd, J=8.8, 2.5 Hz, 1H); 7.17 (d, J=3.4 Hz, 1H); 7.32 (d, J=8.8 Hz, 1H); 7.53 (d, J=2.4 Hz, 1H).

IR (neat) cm⁻¹: 3392, 2927, 1740, 1616, and 1522.

Mass m/z (CI): 489 [M+1].

### EXAMPLE 8

### S)-Methyl-2-methoxy-3-[4-{3-(5-methanesulfonyloxyindol-1-yl)propylamino}phenyl]propanoate

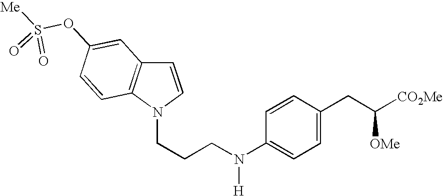

Yield: 675 mg, 76%.

¹H NMR (CDCl₃, 400 MHz) δ: 2.13 (quintet, J=6.9 Hz, 2H); 2.85-2.94 (m, 2H); 3.08 (t, J=6.8 Hz, 2H); 3.13 (s, 3H); 3.35 (s, 3H); 3.72 (s, 3H); 3.91 (dd, J=7.4, 5.3 Hz, 1H); 4.27 (t, J=6.9 Hz, 2H); 6.49 (d, J=8.8 Hz, 2H); 6.52 (d, J=2.5 Hz, 1H); 7.02 (d, J=8.8 Hz, 2H); 7.12 (dd, J=8.8, 2.5 Hz, 1H); 7.18 (d, J=2.4 Hz, 1H); 7.33 (d, J=8.8 Hz, 1H); 7.54 (d, J=2 Hz, 1H).

IR (neat) cm⁻¹: 3404, 2929, 1742, 1616, and 1521.

Mass m/z(CI): 461 [M+1].

### EXAMPLE 9

### Ethyl 2-methyl-2-[4-{3-(5-methanesulfonyloxyindol-1-yl)propylamino}phenoxy]propanoate

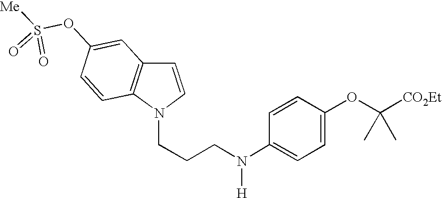

Yield: 600 mg, 54%

¹H NMR (CDCl₃, 400 MHz): 1.28 (t, J=7.1 Hz, 3H); 1.50 (s, 6H); 2.11 (quintet, J=6.3 Hz, 2H); 3.05 (t, J=6.9 Hz, 2H); 3.11 (s, 3H); 4.20-4.27 (m, 4H); 6.42 (d, J=8.8 Hz, 2H); 6.51 (d, J=3 Hz, 1H); 6.76 (d, J=8.8 Hz, 2H); 7.10 (dd, J=8.8, 2.5 Hz, 1H); 7.17 (d, J=3.4 Hz, 1H); 7.31 (d, J=8.8 Hz, 1H); 7.53 (d, J=2 Hz, 1H).

IR (neat) cm⁻¹: 3399, 2935, 1730, 1611, and 1512.

Mass m/z (CI): 475 [M+1].

### EXAMPLE 10

### (S)-Methyl 3-ethoxy-4-[4-{3-(5-methanesulfonyloxyindol-1-yl)propylamino}phenyl]butanoate

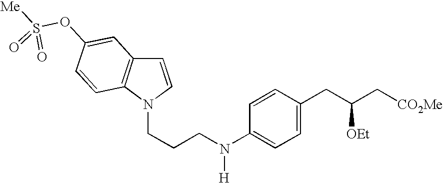

Yield: 500 mg, 49%

¹H NMR (CDCl₃, 400 MHz) δ: 1.12 (t, J=7 Hz, 3H); 2.13 (quintet, J=6.4 Hz, 2H); 2.42 (d, J=2.4 Hz, 1H); 2.43 (d, J=4.5 Hz, 1H); 2.62 (dd, J=14, 7 Hz, 1H); 2.80 (dd, J=14, 5.8 Hz, 1H); 3.08 (t, J=6.8 Hz, 2H); 3.12 (s, 3H); 3.47-3.53 (m, 2H); 3.65 (s, 3H); 3.88 (quintet, J=5.8 Hz, 1H); 4.27 (t, J=6.7 Hz, 2H); 6.48 (d, J=8.8 Hz, 2H); 6.52 (dd, J=3, 0.7 Hz, 1H); 6.99 (d, J=8.8 Hz, 2H); 7.11 (dd, J=8.8, 2.4 Hz, 1H); 7.17 (d, J=3.4 Hz, 1H); 7.32 (d, J=8.8 Hz, 1H); 7.53 (d, J=2.1 Hz, 1H).

IR (neat) cm⁻¹: 3406, 2929, 1734, 1616, 1521.

Mass m/z(CI): 489 [M+1].

### EXAMPLE 11

### Ethyl 2-ethoxy-3-[4-{3-(2,3-dihydroindol-1-yl)propylamino}phenyl]propanoate

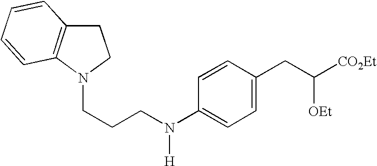

Yield: 465 mg, 35%

¹H NMR (CDCl₃, 400 MHz) δ: 1.17 (t, J=7.3 Hz, 3H); 1.22 (t, J=6.8 Hz, 3H); 1.92 (quintet, J=6.8 Hz, 2H); 2.90 (d, J=6.8 Hz, 2H); 2.96 (t, J=6.9 Hz, 2H); 3.17 (t, J=6.9 Hz, 2H); 3.25 (t, J=6.9 Hz, 2H); 3.30-3.40 (m, 3H); 3.56-3.61 (m, 1H); 3.80 (bs, 1H); 3.94 (t, J=6.9 Hz, 1H); 4.16 (q, J=6.8 Hz, 2H); 6.48 (d, J=7.8 Hz, 1H); 6.54 (d, J=8.3 Hz, 2H); 6.56 (t, J=7.3 Hz, 1H); 7.03-7.09 (aromatics, 4H).

IR (neat) cm⁻¹: 3398, 2926, 1742, 1610, 1522.

Mass m/z(CI): 397 [M+1].

### EXAMPLE 12

### Ethyl 2-ethoxy-3-[4-{(6-methanesulfonyloxy-1,2,3,4-tetrahydronapth-2-yd)methylamino}phenyl]propanoate

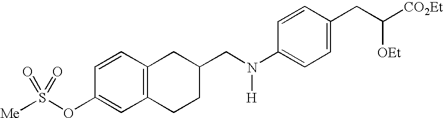

Yield: 100 mg, 20%

¹H NMR (CDCl₃, 200 MHz) δ: 1.17 (t, J=7 Hz, 3H); 1.25 (t, J=7.2 Hz, 3H); 1.42-1.55 (m, 1H); 1.95-2.00 (m, 2H); 2.51 (dd, J=16, 10 Hz, 1H); 2.80-3.00 (m, 5H); 3.12-3.18 (m, 5H); 3.25-3.42 (m, 1H); 3.48-3.65 (m, 1H); 3.94 (t, J=6.6 Hz, 1H); 4.16 (q, J=7.2 Hz, 2H); 6.57 (d, J=8.3 Hz, 2H); 7.90-7.15 (aromatics, 5H).

IR (neat) cm⁻¹: 2925, 1739.

Mass m/z (ES): 476 [M+1].

### EXAMPLE 13

### Ethyl 2-ethoxy-3-[4-{3-(6-methanesulfonyloxy-1,2,3,4-tetrahydronapth-2-yl)propylamino}phenyl]propanoate

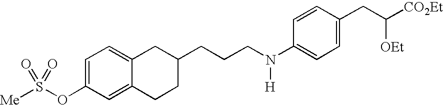

Yield: 125 mg, 16%.

¹H NMR (CDCl₃, 400 MHz) δ: 1.17 (t, J=7.1 Hz, 3H); 1.23 (t, J=7 Hz, 3H); 1.20-1.60 (m, 5H); 1.72 (quintet, J=7.3 Hz, 2H); 1.90-2.00 (m, 1H); 2.40 (dd, J=16, 10 Hz, 1H); 2.80-2.85 (m, 2H, 2.90 (d, J=6.7 Hz, 2H); 3.10-3.14 (m, 5H); 3.33-3.40 (m, 1H); 3.55-3.62 (m, 1H); 3.95 (t, J=6.7 Hz, 1H); 4.17 (q, J=7.0 Hz, 2H); 6.55 (d, J=8.3 Hz, 2H); 6.98-7.09 (aromatics, 5H).

IR (neat) cm⁻¹: 3403, 2926, 1741, 1616, and 1522.

Mass m/z(CI): 504 [M+1].

### EXAMPLE 14

### Ethyl 2-ethoxy-3-[4-{3-(1,2,3,4-tetrahydroquinolyl-1-yl)propylamino}phenyl]propanoate

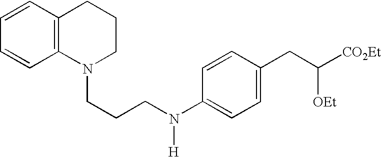

Yield: 455 mg, 43%.

¹H NMR (CDCl₃, 400 MHz) δ: 1.17 (t, J=7 Hz, 3H); 1.22 (t, J=7.2 Hz, 3H); 1.88-1.97 (m, 4H); 2.75 (t, J=6.6 Hz, 2H); 2.89 (d, J=6.8 Hz, 2H); 2.96 (t, J=6.9 Hz, 2H); 3.18 (t, J=6.9 Hz, 2H); 3.27 (t, J=6.9 Hz, 2H); 3.32-3.39 (m, 3H); 3.55-3.62 (m, 1H+NH); 3.94 (t, J=6.9 Hz, 1H); 4.16 (q, J=7.2 Hz, 2H); 6.52 (d, J=8 Hz, 2H); 6.54-6.59 (aromatics, 2H); 6.94 (dd, J=7.3, 1.5. Hz, 1H); 7.00-7.05 (aromatics, 3H).

IR (neat) cm⁻¹: 3392, 2929, 1738, 1520.

Mass m/z(CI): 411 [M+1].

### EXAMPLE 15

### Ethyl 2-methyl-2-[4-{6-methanesulfonyloxynapth-2-ylmethoxy}phenoxy]propanoate

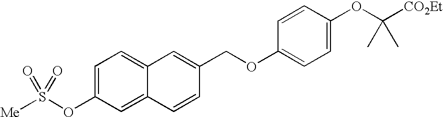

A mixture of Ethyl 2-methyl-2-(4-hydroxyphenoxy)propanoate (200 mg, 1 eq, 0.89 mmol), (Ref: J. Med. Chem. 2001, 44, 2061) (0.350 g) 6-(methanesulfonyloxy)napth-2-ylmethyl bromide (280 mg, 1 eq, 0.89 mmol), obtained in preparation 2, and anhydrous K₂CO₃ (368 mg, 3 eq, 2.67 mmol) in 5 mL dry DMF was stirred at RT for 17 h. Reaction mixture was diluted with ethyl acetate (100 mL), and washed with water (2×100 mL). Organic layer was dried Na₂SO₄), condensed, and the residue was chromatographed using ethyl acetate and hexane to obtain the title compound.

Yield: 335 mg, 82%.

¹H NMR (CDCl₃, 400 MHz) δ: 1.26 (t, J=7.2 Hz, 3H); 1.54 (s, 6H); 3.18 (s, 3H); 4.23 (q, J=7.2 Hz, 2H); 5.17 (s, 2H); 6.83-6.89 (aromatics, 4H); 7.41(dd, J=8.8, 2.4 Hz, 1H); 7.58 (dd, J=8.8, 1.6 Hz, 1H); 7.66 (d, J=2.4 Hz, 1H); 7.85-7.90 (aromatics, 3H)

IR (neat) cm⁻¹: 2986, 2936, 1730, and 1503.

Mass m/z (CI): 459 [M+1].

### EXAMPLE 16

### Ethyl 2-methyl-2-[4-{3-(5-methanesulfonyloxyindol-1-yl)propyloxy}phenoxy]propanoate

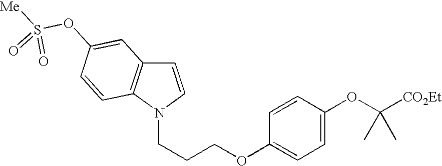

The compound was made using the typical procedure described for example 15 except that the reaction mixture was heated at 70° C. for 4 h.

Yield: 410 mg, 57%.

¹H NMR (CDCl₃, 400 MHz) δ: 1.28 (t, J=7.1 Hz, 3H); 1.54 (s, 6H); 2.24 (quintet, J=6.1 Hz, 2H); 3.10 (s, 3H); 3.80 (t, J=5.7 Hz, 2H); 4.24 (q, J=7.1 Hz, 2H); 4.35 (t, J=6.6 Hz, 2H); 6.48 (d, J=3 Hz, 1H); 6.74 (d, J=9.1 Hz, 2H); 6.90 (d, J=9.1 Hz, 2H); 7.08 (dd, J=8.8, 2.5 Hz, 1H); 7.15 (d, J=3 Hz, 1H); 7.33 (d, J=8.8 Hz, 1H); 7.52 (d, J=2.4 Hz, 1H).

IR (neat) cm⁻¹: 2938, 1732, 1609, and 1505.

### EXAMPLE 17

### Ethyl 2-methyl-2-[4-{3-(5-methanesulfonyloxyindol-1-yl)propyl}phenoxy]propanoate

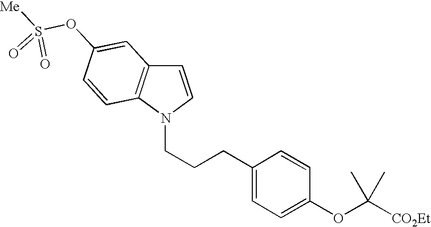

To a stirred solution of 5-methanesulfonyloxyindole (300 mg, 1 eq, 0.87 mmol), obtained in step 1 of preparation 7, and powdered KOH (50 mg, 1 eq, 0.87 mmol) in dry DMSO (4 mL) at RT for 20 min, ethyl 2-methyl-2-[4-(3-methanesulfonyloxypropyl)phenoxy]propionate (219 mg, 1.2 eq, 1.04 mmol), obtained in preparation 16, in 1 mL of dry DMSO was added at RT. And the reaction was stirred at RT for 3 h. Reaction mixture was diluted with ethyl acetate (100 mL), and washed with water (2×100 mL). Organic layer was dried (Na₂SO₄), condensed, and the residue was chromatographed using ethyl acetate and hexane to obtain the title compound.

Yield: 350 mg, 87%.

¹H NMR (CDCl₃, 400 MHz) δ: 1.25 (t, J=7.3 Hz, 3H); 1.57 (s, 6H); 2.14 (quintet, J=7.3 Hz, 2H); 2.56 (t, J=7.3 Hz, 2H); 3.12 (s, 3H); 4.10 (t, J=7 Hz, 2H); 4.24 (q, J=7.3 Hz, 2H); 6.50 (d, J=2.5 Hz, 1H); 6.79 (d, J=8.8 Hz, 2H); 7.02 (d, J=8.8 Hz, 2H); 7.11-7.15 (aromatics, 2H); 7.23 (d, J=8.8 Hz, 1H); 7.52 (d, J=3 Hz, 1H).

IR (neat) cm⁻¹: 2937, 1731, 1611, and 1509.

Mass m/z(CI): 460 [M+1].

### EXAMPLE 18

### Ethyl 2-methyl-2-[4-{3-(3,4-dihydro-2H-bezo[b][1,4]Oxazin-4-yl)propyl}phenoxy]propanoate

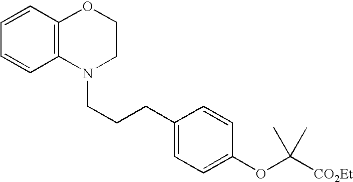

A mixture of 3,4-dihydro-2H-benzo[b][1,4]oxazine (204 mg, 1 eq, 1.51 mmol), ethyl 2-methyl-2-[4-(3-iodopropyl)phenoxy]propanoate (570 mg, 1 eq, 1.51 mmol), obtained in preparation 18, and anhydrous K₂CO₃ (625 mg, 3 eq, 4.53 mmol) in dry DMF (8 mL) was stirred at 70° C. for 17 h. Reaction mixture was diluted with ethyl acetate (100 mL), and washed with water (2×100 mL). Organic layer was dried (Na₂SO₄), condensed, and the residue was chromatographed using ethyl acetate and hexane to obtain the title compound.

Yield: 170 mg, 30%.

Mass m/z(CI): 484 [M+1. The following examples (examples 19-22) were made following the typical procedure of example 18.

### EXAMPLE 19

### Ethyl 2-methyl-2-[4-{3-(3-methanesulfonyloxyphenoxy)propyl}phenoxy]propanoate

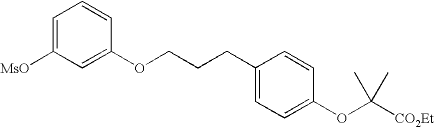

Yield: 500 mg, 66%.

¹H NMR (CDCl₃, 400 MHz) δ: 1.25 (t, J=7 Hz, 3H); 1.57 (s, 6H); 2.04-2.08 (m, 2H); 2.73 (t, J=7.3 Hz, 2H); 3.13 (s, 3H); 3.94 (t, J=6.1 Hz, 2H); 4.23 (q, J=7 Hz, 2H); 6.78 (d, J=8.8 Hz, 2H); 6.80-6.87 (aromatics, 3H); 7.06 (d, J=8.8 Hz, 2H); 7.28 (dd, J=8.6, 8.0 Hz, 1H).

IR (neat) cm⁻¹: 2939, 1732, 1608, 1508.

Mass m/z (ES): 437 [M+1], 454 [M+18], 459 [M+23].

### EXAMPLE 20

### Ethyl 2-methyl-2-[3-{3-(4-methanesulfonyloxyphenoxy)propyl}phenoxy]propanoate

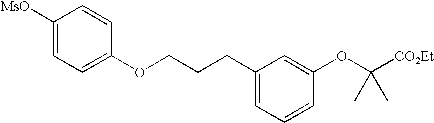

Yield: 400 mg, 73%.

¹H NMR (CDCl₃, 400 MHz) δ: 1.24 (t, J=7.2 Hz, 3H); 1.58 (s, 6H); 2.04-2.10 (m, 2H); 2.74 (t, J=7.1 Hz, 2H); 3.10 (s, 3H); 3.93 (t, J=6.2 Hz, 2H); 4.22 (q, J=7.2 Hz, 2H); 6.66 (dd, J=8.1, 2.4 Hz, 1H); 6.73 (d, J=2.0 Hz, 1H); 6.83 (d, J=7.5 Hz, 1H); 6.88 (d, J=9.1 Hz, 2H); 7.14 (t, J=7.8 Hz, 1H); 7.18 (d, J=9.1 Hz, 2H).

IR (neat) cm⁻¹: 2928, 1732, 1608, 1502.

Mass m/z (CI): 437 [M+1].

### EXAMPLE 21

### Ethyl 2-methyl-2-[4-{3-(4-Methanesulfonyloxyphenoxy)propyloxy)phenoxy]propanoate

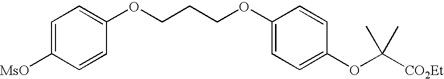

Yield: 218 mg, 41%.

¹H NMR (CDCl₃, 200 MHz) δ: 1.27 (t, J=7 Hz, 3H); 1.53 (s, 6H); 2.23 (quintet, J=6 Hz, 2H); 3.10 (s, 3H); 4.06-4.17 (m, 4H); 4.24 (q, J=7 Hz, 2H); 6.74-6.94 (aromatics, 6H); 7.19 (d, J=9 Hz, 2H).

IR (neat) cm⁻¹: 2934, 1729, 1593, 1501.

Mass m/z (CI): 453 [M+1].

### EXAMPLE 22

### Ethyl 2-methyl-2-[3-{3-(3-methanesulfonyloxyphenoxy)propyloxy}phenoxy]propanoate

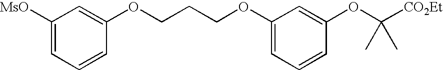

Yield: 500 mg, 68%.

¹H NMR (CDCl₃, 400 MHz) δ: 1.24 (t, J=7.2 Hz, 3H); 1.59 (s, 6H); 2.24 (quintet, J=6.2 Hz, 2H); 3.12 (s, 3H); 4.10 (t, J=6 Hz, 2H); 4.14 (t, J=6.1 Hz, 2H); 4.22 (q, J=7.2 Hz, 2H); 6.39-6.56 (aromatics, 3H); 6.83-6.88 (aromatics, 3H); 7.11 (t, J=8 Hz, 1H); 7.29 (t, J=8.2 Hz, 1H).

IR (neat) cm⁻¹: 2936, 1732, 1603, 1486.

Mass m/z (CI): 453 [M+1].

### EXAMPLE 23

### (S)-2-Methoxy-3-[4-{6-methanesulfonyloxynapth-1-ylmethylamino}phenyl]propanoic acid

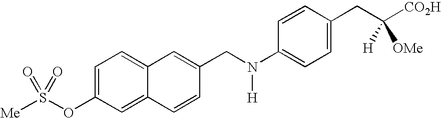

Ethyl 2-methoxy-3-[4-{3-(4-methanesulfonyloxyphenyl)propylamino}phenyl]propanoate (400 mg, 1.0 eq, 0.875 mmol), obtained in example 1, was hydrolyzed by treating with LiOH.H₂O (55.1 mg, 1.5 eq, 1.31 mmol) in MeOH-THF-water solvent mixture at RT for 34 h. The reaction mixture was condensed, diluted with water and acidified (pH at 3-4) with aq. HCl. Desired acid was extracted from aqueous layer, dried (Na₂SO₄), condensed, which was then chromatographed using MeOH and CHCl₃ as eluents to obtain the pure acid as thick mass (150 mg, 40% yield).

¹H NMR (CDCl₃, 400 MHz) δ: 2.92 (dd, J=14.2, 7.4 Hz, 1H); 3.05 (dd, J=14.2, 4.4 Hz, 1H); 3.18 (s, 3H); 3.40 (s, 3H); 3.97 (dd, J=7.4, 4.4 Hz, 1H); 4.49 (s, 2H); 6.62 (d, J=8.3 Hz, 2H); 7.04 (d, J=8.3 Hz, 2H); 7.39 (dd, J=8.8, 2.4 Hz, 1H); 7.55 (dd, J=8.3, 1.4 Hz, 1H); 7.74 (d, J=2 Hz, 1H); 7.80-7.85 (aromatics, 3H).

IR (neat) cm⁻¹: 3436, 2927, 1730, 1616, and 1519.

Mass m/z(ES): 430 [M+1], 452 [M+23]. The following examples (examples 2444) were made using the typical procedure described for example 23.

### EXAMPLE 24

### 2-Ethoxy-3-[4-{6-methanesulfonyloxynapth-2-ylmethylamino}phenyl]propanoic acid

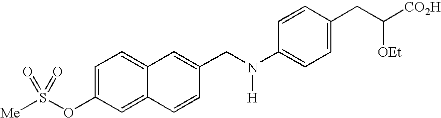

Mp: 168-170° C. Yield: 120 mg, 42%.

¹H NMR (CDCl₃, 200 MHz) δ: 1.15 (t, J=7 Hz, 3H); 2.87 (dd, J=14.1, 7.8 Hz, H); 2.96 (dd, J=14.1, 4.3 Hz, 1H); 3.16 (s, 3H); 3.22-3.42 (m, 1H); 3.48-3.68 (m, 1H); 3.93 (dd, J=7.8, 4.3 Hz, 1H); 4.50 (s, 2H); 6.59 (d, J=8.3 Hz, 2H); 7.07 (d, J=8.3 Hz, 2H); 7.37 (dd, J=8.8, 2.7 Hz, 1H); 7.57 (dd, J=8.8, 1.6 Hz, 1H); 7.75 (d, J=2.7 Hz, 1H); 7.82-7.87 (aromatics, 3H).

IR (neat) cm⁻: 34241, 2924, and 1516.

Mass m/z (CI): 444 [M+1], 466 [M+23].

### EXAMPLE 25

### 2-Ethoxy-5-[4-{6-methanesulfonyloxynapth-2-ylmethylamino}phenyl]pentatonic acid

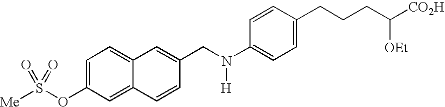

Yield: 180 mg, 64%.

¹H NMR (CDCl₃, 400 MHz): 1.23 (t, J=7.3 Hz, 3H); 1.62-1.82 (m, 4H); 2.52(t, J=7.3 Hz, 2H); 3.18 (s, 3H); 3.47-3.55 (m, 1H); 3.58-3.64 (m, 1H); 3.88 (t, J=5.4 Hz, 1H); 4.48 (s, 2H); 6.59 (d, J=8.8 Hz, 2H); 6.97 (d, J=8.8 Hz, 2H); 7.38 (dd, J=8.8, 2.4 Hz, 1H); 7.55 (d, J=9.7 Hz, 1H); 7.74 (d, J=1.9 Hz, 1H); 7.81-7.86 (aromatics, 3H).

IR (neat) cm⁻: 3409, 2926, 1724, 1613, and 1520.

Mass m/z (CI): 472 [M+1], 494 [M+23], 943 [M₂+1]

### EXAMPLE 26

### 2-Methyl-2-[4-(6-methanesulfonyloxynapth-2-ylmethylamino}phenoxy]propanoic acid

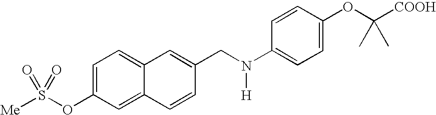

Mp: 182-184° C. Yield: 220 mg, 47%.

¹H NMR (CDCl₃, 400 MHz) δ: 1.44 (s, 6H); 3.26 (s, 3H); 4.44 (s, 2H); 6.52 (d, J=8.8 Hz, 2H); 6.74 (d, J=8.8 Hz, 2H); 7.40 (dd, J=8.8, 2.4 Hz, 1H); 7.58 (dd, J=8.8, 1.2 Hz, 1H); 7.76 (s, 1H); 7.83-7.88 (aromatics, 3H).

IR (KBr) cm⁻¹: 3428, 2924, 2854, 1714, and 1515.

Mass m/z (ES): 430.1[M+1], 452.1 [M+Na], 859.5 [M₂+1].

### EXAMPLE 27

### 2-Ethoxy-3-[4-{3-(indol-1-yl)propylamino}phenyl]propanoic acid

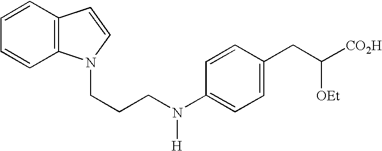

Yield: 180 mg, 71%.

¹H NMR (CDCl₃, 400 MHz) δ: 1.18 (t, J=7.0 Hz, 3H); 2.14 (quintet, J=6.8 Hz, 2H); 2.89 (dd, J=14.1, 7.7 Hz, 1H); 3.03 (dd, J=14.1, 4.4 Hz, 1H); 3.08 (t, J=7 Hz, 2H); 3.44-3.50 (m, 1H); 3.55-3.60 (m, 1H); 4.03 (dd, J=7.4, 4.4 Hz, 1H); 4.27 (t, J=6.9, 2H); 6.48 (d, J=8.8 Hz, 2H); 6.50 (dd, J=10, 4 Hz, 1H); 7.03 (d, J=8.3 Hz, 2H); 7.08-7.12 (aromatics, 2H); 7.20 (dt, J=8.3, 1.5 Hz, 1H); 7.34 (d, J=8.3 Hz, 1H); 7.64 (d, J=7.8 Hz, 1H).

IR (neat) cm⁻¹: 3391, 2925, 1726, 1613, and 1519.

Mass m/z(CI): 367 (M+1].

### EXAMPLE 28

### (S)-2-Methoxy-3-[4-{3-(indol-1-yl)propylamino}phenyl]propanoic acid

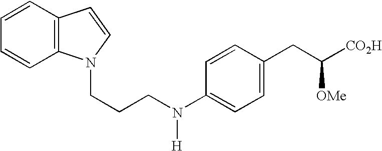

Yield: 190 mg, 58%.

¹H NMR (CDCl₃, 400 MHz) δ: 2.14 (quintet, J=6.8 Hz, 2H); 2.92 (dd, J=14.1, 7.3 Hz, 1H); 3.04 (dd, J=14.1, 4.4 Hz, 1H); 3.09 (t, J=6.9 Hz, 2H); 3.39 (s, 3H); 3.96 (dd, J=7.3, 4.4 Hz, 1H); 4.26 (t, J=6.8, 2H); 6.46-6.52 (aromatics, 3H); 7.05 (d, J=8.3 Hz, 2H); 7.08-7.13 (aromatics, 2H); 7.21 (dt, J=8.3, 1.5 Hz, 1H); 7.35 (d, J=8.3 Hz, 1H); 7.64 (d, J=7.8 Hz, 1H).

IR (neat) cm⁻¹: 3400, 2929, 1727, 1614, 1517.

Mass m/z (ES): 353 [M+1], 375 [M+23].

### EXAMPLE 29

### (S)-2-Ethoxy-3-[4-{3-(5-methanesulfonyloxyindol-1-yl)propylamino}phenyl]propanoic acid

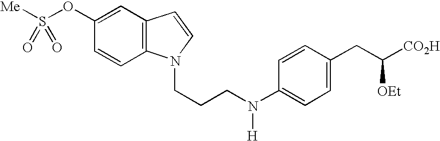

Yield: 400 mg, 71%.

¹H NMR (CDCl₃, 400 MHz) δ: 1.19 (t, J=7 Hz, 3H); 2.13 (quintet, J=6.9 Hz, 2H); 2.90 (dd, J=14.1, 7.3 Hz, 1H); 2.97 (dd, J=14.1, 4.4 Hz, 1H); 3.08 (t, J=6.8 Hz, 2H); 3.12 (s, 3H); 3.44-3.62 (m, 2H); 4.04 (dd, J=7.3, 4.4 Hz, 1H); 4.26 (t, J=7 Hz, 2H); 6.47 (d, J=8.8 Hz, 2H); 6.52 (d, J=2.5 Hz, 1H); 7.03 (d, J=8.3 Hz, 2H); 7.12 (dd, J=8.8, 2.5 Hz, 1H); 7.17 (d, J=3.4 Hz, 1H); 7.31 (d, J=8.8 Hz, 1H); 7.53 (d, J=2.4 Hz, 1H).

IR (neat) cm⁻¹: 3400, 2930, 1729, 1615, and 1520.

Mass m/z (ES): 461 [M+1], 483 [M+23].

### EXAMPLE 30

### (S)-2-Methoxy-3-[4-{3-(5-methanesulfonyloxyindol-1-yl)propylamino}phenyl]propanoic acid

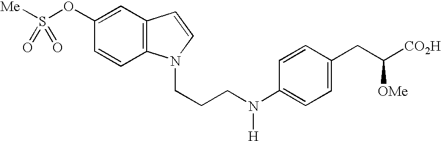

Yield: 420 mg, 65%.

¹H NMR (CDCl₃, 400 MHz): 2.11 (quintet, J=6.9 Hz, 2H); 2.91 (dd, J=14.2, 6.8 Hz, 1H); 3.02 (dd, J=14.2, 4.4 Hz, H); 3.07 (t, J=6.8 Hz, 2H); 3.11 (s, 3H); 3.39 (s, 3H); 3.95 (dd, J=6.8, 4.4 Hz, 1H); 4.25 (t, J=6.9 Hz, 2H); 6.47 (d, J=8.8 Hz, 2H); 6.51 (d, J=2.5 Hz, 1H); 7.02 (d, J=8.8 Hz, 2H); 7.10 (dd, J=8.8, 2.5 Hz, 1H); 7.16 (d, J=2.4 Hz, 1H); 7.30 (d, J=8.8 Hz, 1H); 7.52 (d, J=2.4 Hz, 1H).

IR (neat) cm⁻¹: 3381, 2930, 1732, 1614, and 1521.

### EXAMPLE 31

### 2-Methyl-2-[4-{3-(5-methanesulfonyloxyindol-1-yl)propylamino}phenoxy]propanoic acid

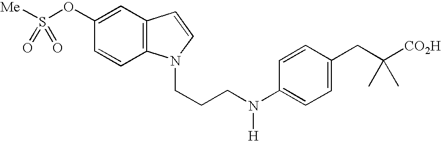

Mp: 164-166° C. Yield: 300 mg, 58%.

¹H NMR(CDCl₃+DMSO-d₆, 400 MHz) δ: 1.43 (s, 6H); 2.09 (quintet, J=6.7 Hz, 2H); 2.99 (t, J=6.8 Hz, 2H); 3.19 (s, 3H); 4.30 (t, J=6.9 Hz, 2H); 6.44 (d, J=8.8 Hz, 2H); 6.48 (d, J=3.2 Hz, 1H); 6.73 (d, J=8.8 Hz, 2H); 7.07 (dd, J=8.8, 2.7 Hz, 1H); 7.33 (d, J=3.2 Hz, 1H); 7.43 (d, J=8.8 Hz, 1H); 7.49 (d, J==2.5 Hz, 1H).

IR (neat) cm⁻: 3400, 2932, 1590, 1611, and 1510.

Mass m/z ES): 447 [M+1], 469 [M+23], 893 [M₂+1].

### EXAMPLE 32

### (S)-3-Ethoxy-4-[4-{3-(5-methanesulfonyloxyindol-1-yl)propylamino}phenyl]butanoic acid

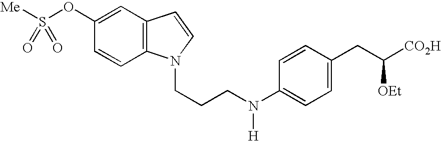

Yield: 300 mg, 61%.

¹H NMR (CDCl₃, 400 MHz) δ: 1.18 (t, J=7 Hz, 3H); 2.13 (quintet, J=6.4 Hz, 2H); 2.45-2.49 (m, 2H); 2.63 (dd, J=14, 7 Hz, 1H); 2.86 (dd, J=14, 5.8 Hz, 1H); 3.09 (t, J=6.9 Hz, 2H); 3.12 (s, 3H); 3.52-3.63 (m, 2H); 3.84-3.87 (m, 1H); 4.27 (t, J=6.8 Hz, 2H); 6.48 (d, J=8.8 Hz, 2H); 6.52 (d, J=3.4 Hz, 1H); 6.98 (d, J=8.8 Hz, 2H); 7.12 (dd, J=8.8, 2.4 Hz, 1H); 7.18 (d, J=3 Hz, 1H); 7.32 (d, J=8.8 Hz, 1H); 7.54 (d, J=2.1 Hz, 1H).

IR (neat) cm⁻¹: 3384, 2933, 1712, 1615, and 1520.

Mass m/z (ES): 475 [M+1], 497 [M+23].

### EXAMPLE 33

### 2-Ethoxy-3-[4-{3-(2,3-dihydroindol-1-yl)propylamino}phenyl]propanoic acid

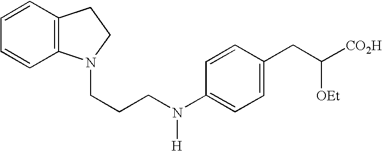

Yield: 280 mg, 72%.

¹H NMR (CDCl₃, 400 MHz) δ: 1.17 (t, J=7.3 Hz, 3H); 1.92 (quintet, J=6.8 Hz, 2H); 2.89 (dd, J=14.2, 7.8 Hz, 1H); 2.96 (t, J=8.3 Hz, 2H); 3.02 (dd, J=14.2, 3.9 Hz, 1H); 3.17 (t, J=6.9 Hz, 2H); 3.24 (t, J=6.9 Hz, 2H); 3.34 (t, J=8.3 Hz, 2H); 3.42-3.50 (m, 1H); 3.53-3.61 (m, 1H); 4.02 (dd, J=7.8, 3.9 Hz, 1H); 6.48 (d, J=7.8 Hz, 1H); 6.55 (d, J=8.3 Hz, 2H); 6.66 (dt, J=7.3, 1 Hz, 1H); 7.03-7.09 (aromatics, 4H).

IR (neat) cm⁻¹: 3391, 2927, 1725, 1607, and 1520.

Mass m/z (CI): 369 [M+1].

### EXAMPLE 34

### 2-Ethoxy-3-[4-{(6-methanesulfonyloxy-1,2,3,4-tetrahydronapth-2-yl)methylamino}phenyl]propanoic acid

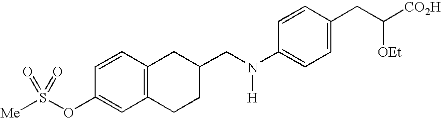

Yield: 55 mg, 58%.

¹H NMR (CDCl₃, 400 MHz) δ: 1.19 (t, J=7 Hz, 3H); 1.42-1.55 (m, 1H); 2.00-2.15 (m, 2H); 2.52 (dd, J=16, 10 Hz, 1H); 2.80-3.15 (m, 7H); 3.12 (s, 3H); 3.42-3.60 (m, 2H); 4.04 (dd, J=7.3, 4.3 Hz, 1H); 6.58 (d, J=8.3 Hz, 2H); 7.03-7.12 (aromatics, 5H).

IR (neat) cm⁻¹: 3500, 2927, and 1728.

Mass m/z (ES): 448 [M+1], 470 [M+23].

### EXAMPLE 35

### 2-Ethoxy-3-[4-{3-(6-methanesulfonyloxy-1,2,3,4-tetrahydronapth-2-yl)propylamino}phenyl]propanoic acid

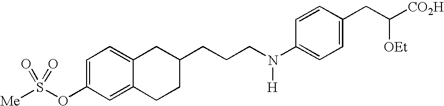

Yield: 75 mg, 69%.

¹H NMR (CDCl₃, 400 MHz) δ: 1.15 (t, J=7 Hz, 3H); 1.20-1.80 (m, 7H); 1.82-2.00 (m, 1H); 2.40 (dd, J=16, 10 Hz, 1H); 2.75-2.85 (m, 2H); 2.85-3.10 (m, 2H); 3.10-3.20 (m, 4H); 3.45-3.55 (m, 1H); 3.55-3.70 (m, 2H); 4.0 5 (dd, J=7.4, 4.4 Hz, 1H); 6.68 (d, J=8.3 Hz, 2H); 6.98-7.09 (aromatics, 5H).

IR (neat) cm⁻¹: 3503, 2928, and 1694.

Mass m/z (CI): 476 [M+1], 498 [M+23].

### EXAMPLE 36

### 2-Ethoxy-3-[4-{3-(1,2,3,4-tetrahydroquinolyn-1-yl)propylamino}phenyl]propanoic acid

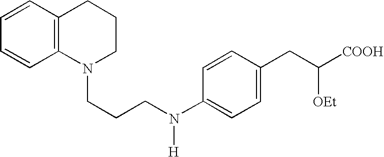

Yield: 215 mg, 58%.

¹H NMR (CDCl₃, 400 MHz) δ: 1.18 (t, J=7 Hz, 3H); 1.90-1.96 (m, 4H); 2.75 (t, J=7 Hz, 2H); 2.89 (dd, J=14, 7 Hz, 1H); 3.03 (dd, J=14, 4 Hz, 1H); 3.18 (t, J=7 Hz, 2H); 3.27 (t, J=6.9 Hz, 2H); 3.37 (t, J=7 Hz, 2H); 3.42-3.50 (m, 1H); 3.50-3.60 (m, 1H); 4.02 (dd, J=7, 4 Hz, 1H); 6.53-6.59 (aromatics, 4H); 6.94 (d, J=7.3 Hz, 1H); 7.00-7.06 (aromatics, 3H).

IR (neat) cm⁻¹: 3400, 2928, 1725, 1601.

Mass m/z(CI): 383 [M+1].

### EXAMPLE 37

### 2-Methyl-2-[4-{6-methanesulfonyloxynapth-2-ylmethoxy}phenoxy]propanoic acid

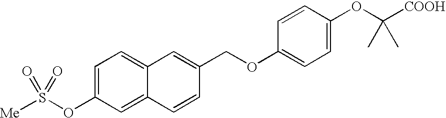

Mp: 147-149° C. Yield: 98 mg, 36%.

¹H NMR (CDCl₃, 400 MHz)) δ: 1.53 (s, 6H); 3.22 (s, 3H); 5.18 (s, 2H); 6.86-6.93 (aromatics, 4H); 7.40-7.43(aromatics, 2H); 7.60 (d, J=8 Hz, 1H); 7.86-7.92 (aromatics, 3H)

IR (neat) cm⁻¹: 3430, 2924, 1715, and 1504.

Mass m/z (CI): 448.3 [M+NH₄], 878.5 [M₂+NH₄].

### EXAMPLE 38

### 2-Methyl-2-[4-{3-(5-methanesulfonyloxyindol-1-yl)propyloxy}phenoxy]propanoic acid

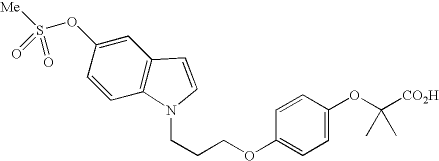

Yield: 300 mg, 85%.

¹H NMR (CDCl₃, 400 MHz) δ: 1.54 (s, 6H); 2.26 (quintet, J=6 Hz, 2H); 3.12 (s, 3H); 3.82 (t, J=5.6 Hz, 2H); 4.37 (t, J=6.4 Hz, 2H); 6.49 (d, J=3 Hz, 1H); 6.77 (d, J=8.8 Hz, 2H); 6.91 (d, J=8.8 Hz, 2H); 7.08 (dd, J=8.8, 2.4 Hz, 1H); 7.15 (d, J=3.3 Hz, 1H); 7.32 (d, J=8.8 Hz, 1H); 7.51 (d, J=2.1 Hz, 1H).

IR (neat) cm⁻¹: 3400, 2937, 1717, 1611, and 1505.

Mass m/z(ES): 448 [M+1], 470 [M+23].

### EXAMPLE 39

### 2-Methyl-2-[4-{3-(5-methanesulfonyloxyindol-1-yl)propyl}phenoxy]propanoic acid

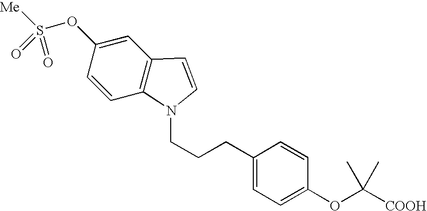

Yield: 170 mg, 52%.

¹H NMR (CDCl₃, 400 MHz) δ: 1.57 (s, 6H); 2.16 (quintet, J=7.3 Hz, 2H); 2.58 (t, J=7.7 Hz, 2H); 3.12 (s, 3H); 4.12 (t, J=7 Hz, 2H); 6.50 (d, J=2.7 Hz, 1H); 6.87 (d, J=8.6 Hz, 2H); 7.05 (d, J=8.6 Hz, 2H); 7.09-7.15 (aromatics, 2H); 7.22 (d, J=8.8 Hz, 1H); 7.52 (d, J=2.4 Hz, 1H).

IR (neat) cm⁻¹: 3326, 2937, 1716, 1609, and 1508.

Mass m/z(CI): 432 [M+1].

### EXAMPLE 40

### 2-Methyl-2-[4-{3-(3,4-dihydro-2H-bezo[b][1,4]Oxazin-4-yl)propyl}phenoxy]propanoic acid

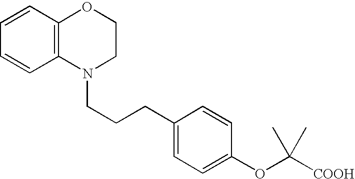

Yield: 70 mg, 45%.

¹H NMR (CDCl₃, 400 MHz) δ: 1.47 (s, 6H); 1.78 (quintet, J=7.5 Hz, 2H); 2.55 (t, J=7.6 Hz, 2H); 3.20-3.30 (m, 4H); 4.13 (t, J=4.3 Hz, 2H); 6.47 (dt, J=Hz, 1H); 6.56 (dd, J=Hz, 1H); 6.63 (dd, J=Hz, 1H); 6.67-6.73 (aromatics, 1H); 6.76 (d, J=8.6 Hz, 2H); 7.11 (d, J=8.6 Hz, 2H).

IR (neat) cm⁻¹: 3400, 2932, 1715, 1606, and 1506.

Mass m/z(ES): 356 [M+1].

### EXAMPLE 41

### 2-Methyl-2-[4-{3-(3-methanesulfonyloxyphenoxy)propyl}phenoxy]propanoic acid

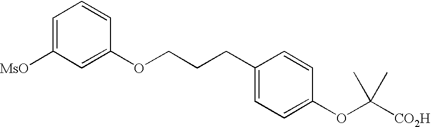

Yield: 260 mg, 56%.

¹H NMR (CDCl₃, 400 MHz) δ: 1.58 (s, 6H); 2.04-2.11 (m, 2H); 2.76 (t, J=7.3 Hz, 2H); 3.13 (s, 3H); 3.95 (t, J=6.2 Hz, 2H); 6.79-6.86 (aromatics, 3H); 6.87 (d, J=8.8 Hz, 2H); 7.11 (d, J=8.8 Hz, 2H); 7.28 (t, J=8.4 Hz, 1H).

IR (neat) cm⁻¹: 3400, 2939, 1717, 1608, 1508.

Mass m/z (ES): 409 [M+1], 426 [M+18], 431 [M+23].

### EXAMPLE 42

### 2-Methyl-2-[3-{3-(4-methanesulfonyloxyphenoxy)propyl}phenoxy]propanoic acid

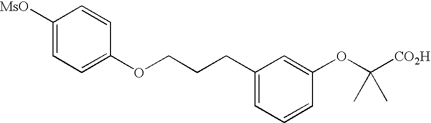

Mp: 93-95° C. Yield: 255 mg, 74%.

¹H NMR (CDCl₃, 400 MHz) δ: 1.57 (s, 6H); 2.04-2.17 (m, 2H); 2.77 (t, J=7.1 Hz, 2H); 3.11 (s, 3H); 3.93 (t, J=6.2 Hz, 2H); 6.75-6.79 (aromatics, 2H); 6.88 (d, J=9.1 Hz, 2H); 6.92 (d, J=7.5 Hz, 1H); 7.17-7.21 (aromatics, 3H):

IR (neat) cm⁻¹: 3375, 2938, 1716, 1585, 1502.

Mass m/z (ES): 409 [M+1], 426 [M+18], 431 [M+23].

### EXAMPLE 43

### 2-Methyl-2-[4-{3-(4-methanesulfonyloxyphenoxy)propyloxy}phenoxy]propanoic acid

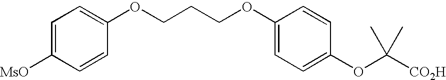

Yield: 115 mg, 58%.

¹H NMR (CDCl₃, 400 MHz) δ: 1.53 (s, 6H); 2.24 (quintet, J=6 Hz, 2H); 3.10 (s, 3H); 4.1.1 (t, J=6.2 Hz, 2H); 4.14 (t, J=6.1 Hz, 2H); 6.81 (d, J=9 Hz, 2H); 6.88-6.93 (aromatics, 4H); 7.19 (d, J=9 Hz, 2H).

IR (neat) cm⁻¹: 3355, 2936, 1718, 1593, 1503.

Mass m/z (ES): 425 [M+1], 442 [M+18], 447 [M+23].

### EXAMPLE 44

### 2-Methyl-2-[3-{3-(3-methanesulfonyloxyphenoxy)propyloxy}phenoxy]propanoic acid

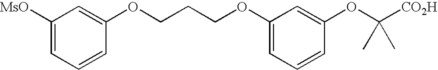

Yield: 250 mg, 59%.

¹H NMR (CDCl₃, 400 MHz) δ: 1.60 (s, 6H); 2.24 (quintet, J=6 Hz, 2H); 3.12 (s, 3H); 4.11 (t, J=6 Hz, 2H); 4.14 (t, J=6.2 Hz, 2H); 6.50-6.62 (aromatics, 3H); 6.85-6.88 (aromatics, 3H); 7.14 (t, J=8 Hz, 1H); 7.29 (t, J=8.3 Hz, 1H).

IR (neat) cm⁻¹: 2936, 1732, 1603, 1486.

Mass m/z (ES): 425 [M+1], 442 [M+18], 866 [M₂+18].

### EXAMPLE 45

### (S)-2-Methoxy-3-[4-{6-methanesulfonyloxynapth-2-ylmethylamino}phenyl]propanoic acid Arginine salt

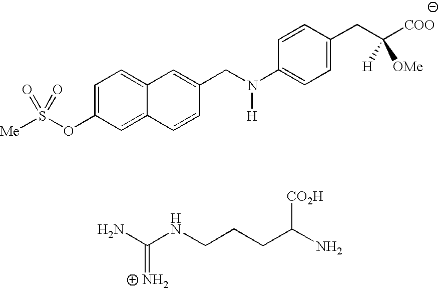

(S)-2-methoxy-3-[4-{6-methanesulfonyloxynapth-2-ylmethylamino}phenyl]propanoic acid (100 mg, 1 eq, 0.233 mmol) obtained in example 23, and L-Arginine (40.6 mg, 1 eq, 0.233 mmol) were taken in dry methanol (3 ml), and stirred at RT for 2-3 h. The solvent was removed on rotavapor followed by benzene azeotrope. The residue was dried under high vacuum pump to yield the title compound as a free flowing white solid (138 mg, yield 100%).

Mpt: 122-124° C.

The following examples (examples 46-61) were made using the typical procedure described for example 45.

### EXAMPLE 46

### 2-Ethoxy-5-[4-{6-methanesulfonyloxynapth-2-ylmethylamino}phenyl]pentatonic acid Arginine salt

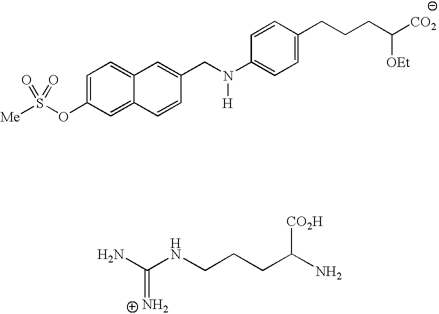

Mp: 118-120° C.

### EXAMPLE 47

### 2-Ethoxy-3-[4-{3-(indol-1-yl)propyl amino}phenyl]propanoic acid Arginine salt

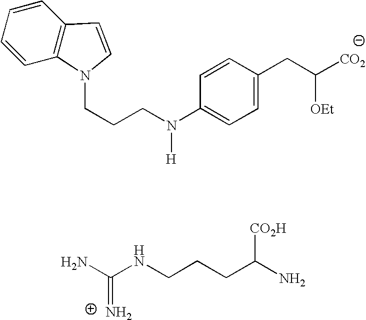

Mp: 130° C.

### EXAMPLE 48

### (S)-2-Methoxy-3-[4-{3-(indol-1-yl)propyl amino}phenyl]propanoic acid Arginine salt

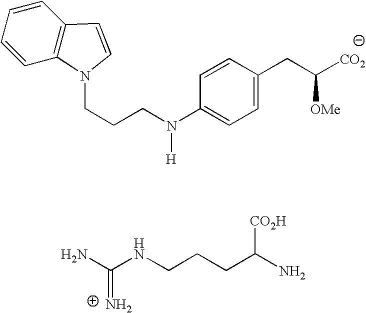

Mp: 105° C.

### EXAMPLE 49

### (S)-2-Ethoxy-3-[4-{3-(5-methanesulfonyloxyindol-1-yl)propylamino}phenyl]propanoic acid Arginine salt

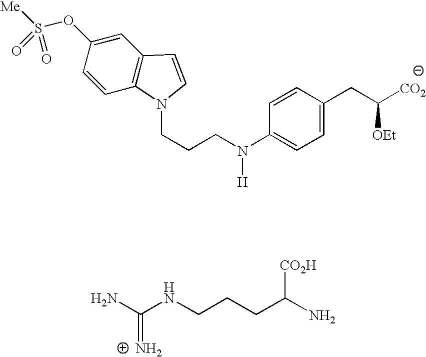

Mp: 102-104° C.

### EXAMPLE 50

### (S)-2-Methoxy-3-[4-{3-(5-methanesulfonyloxyindol-1-yl)propylamino}phenyl]propanoic acid Arginine salt

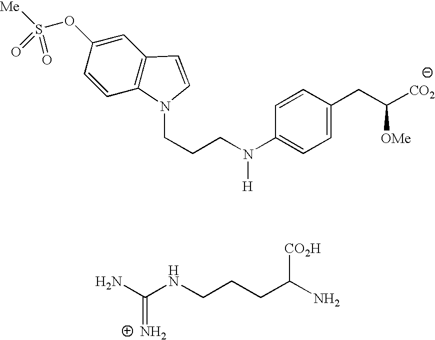

Mp: 102-104° C.

### EXAMPLE 51

### (S)-3-Ethoxy-4-[4-{3-(5-methanesulfonyloxyindol-1-yl)propylamino}phenyl]butanoic acid Arginine salt

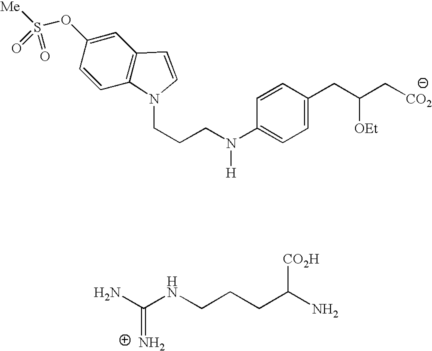

Mp: 98-100° C.

### EXAMPLE 52

### 2-Ethoxy-3-[4-{3-2,3-dihydroindol-1-yl)propylamino}phenyl]propanoic acid Arginine salt

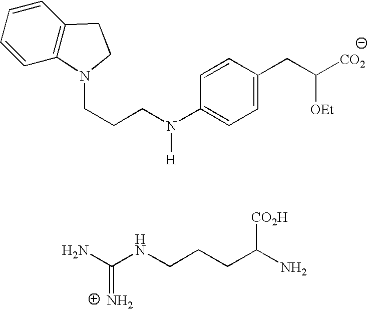

Mp: 130-132° C.

### EXAMPLE 53

### 2-Ethoxy-3-[4-{(6-methanesulfonyloxy-1,2,3,4-tetrahydronapth-2-yl)methylamino}phenyl]propanoic acid Arginine salt

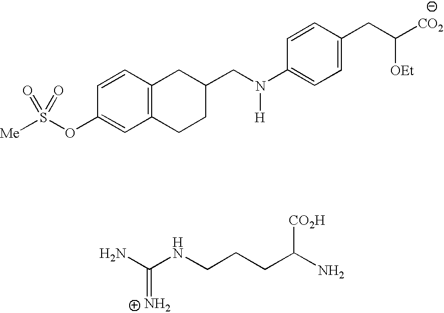

Mpt: 96-98° C.

### EXAMPLE 54

### 2-Ethoxy-3-[4-{3-(6-methanesulfonyloxy-1,2,3,4-tetrahydronapth-2-yl)propylamino}phenyl]propanoic acid Arginine salt

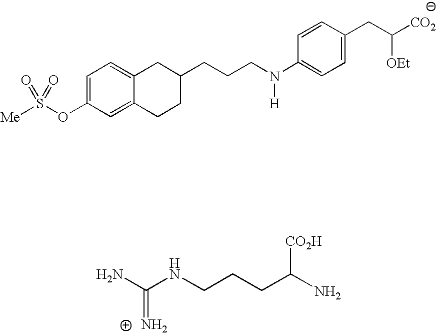

Mp: 115-117° C.

### EXAMPLE 55

### 2-Ethoxy-3-[4-{3-1,2,3,4-tetrahydroquinolyn-1-yl)propylamino}phenyl]propanoic acid Arginine salt

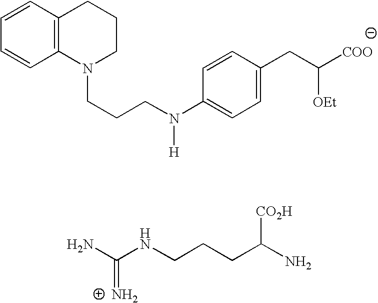

Mp: 134-136° C.

### EXAMPLE 56

### 2-Methyl-2-[4-{3-(5-methanesulfonyloxyindol-1-yl)propyloxy}phenoxy]propanoic acid Arginine salt

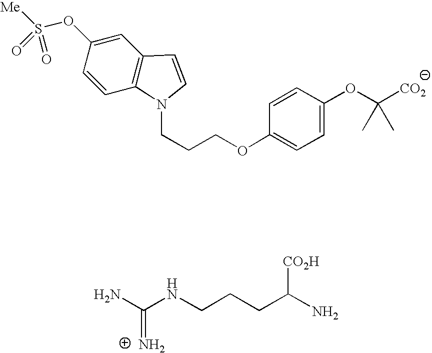

Mp: 125° C.

### EXAMPLE 57

### 2-Methyl-2-[4-({3-(5-methanesulfonyloxyindol-1-yl)propyl}phenoxy]propanoic acid Arginine salt

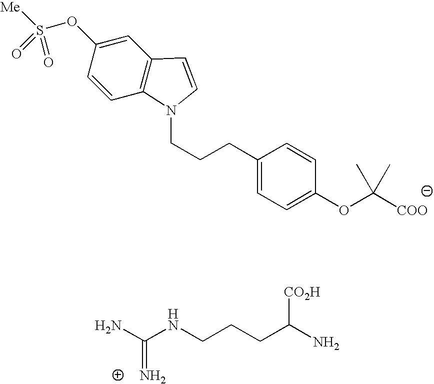

Mp: 80° C.

Mass m/z (ES): 606 [M+1].

### EXAMPLE 58

### 2-Methyl-2-[4-{3-(3,4-dihydro-2H-bezo[b][1,4]Oxazin-4-yl)propyl}phenoxy]propanoic acid Arginine salt

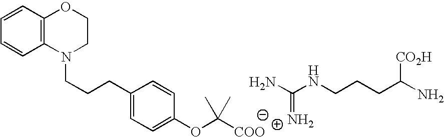

Mp: 78° C.

### EXAMPLE 59

### 2-Methyl-2-[4-{3-(3-methanesulfonyloxyphenoxy)propyl}phenoxy]propanoic acid Arginine salt

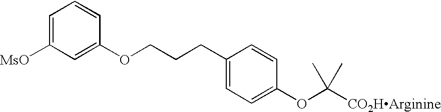

Mp: 95-97° C.

### EXAMPLE 60

### 2-Methyl-2-[4-{3-(4-methanesulfonyloxyphenoxy)propyloxy}phenoxy]propanoic acid Arginine salt

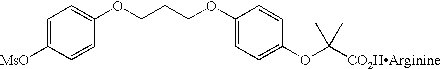

Mp: 82-84° C.

Mass m/z (ES): 599 [M+1].

### EXAMPLE 61

### 2-Methyl-2-[3-{3-(3-methanesulfonyloxyphenoxy)propyloxy}phenoxy]propanoic acid Arginine salt

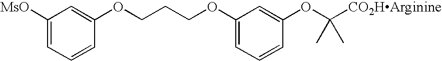

M.p: 110-112° C.

### EXAMPLE 62

### Ethyl 2-{4-[3-(biphenyl-4-yloxy)-propyl]-phenoxy}-2-methyl-propanoate

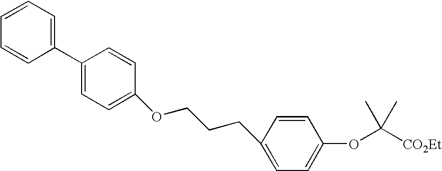

To 4-phenylphenol (400 mg, 2.35 mmol) dissolved in DMF (10 mL) was added K₂CO₃ (973 mg, 7.05 mmol) and stirred at room temperature for 15 min. and then was added ethyl-2-[4-(3-methanesulphonyloxy-propyl)-phenoxy]-2-methyl-propanoate (808 mg, 2.35 mmol) (obtained in preparation 16) in DMF (5 mL) and the mixture was stirred at 80° C. for 12 h and the mixture was cooled to RT and filtered off, washed the K₂CO₃ cake with ethyl acetate (100 mL) the combined filtrates were washed with water thrice and then with brine, dried over Na₂SO₄ and evaporated the ethyl acetate to get a crude product which was purified on silica gel column by eluting with 20% ethyl acetate and hexane to give a thick gum of ethyl 2-{4-[3-(biphenyl-4-yloxy)-propyl]-phenoxy}-2-methyl-propanoate (450 mg, 46%).

¹H NMR (6, CDCl₃, 200 MHz): 7.60-7.20 (m, 7H), 7.08 (d, J=8.55 Hz, 2H), 6.95 (d, J=8.78 Hz, 2H), 6.78 (d, J=8.55 Hz, 2H), 4.23 (q, J=7.08 Hz, 2H), 3.98 (t, J=6.11 Hz, 2H), 2.76 (t, J=7.08 Hz, 2H), 2.20-2.00 (m, 2H), 1.16 (s,6H), 1.28 (t, J=7.08 Hz, 3H).

### EXAMPLE 63

### 2-{4-[3-(Biphenyl-4-yloxy)-propyl]-phenoxy}-2-methyl-propanoic acid

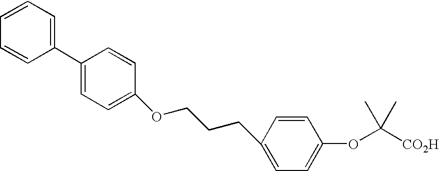

Ethyl 2-{4-[3-(biphenyl-4-yloxy)-propyl]-phenoxy}-2-methyl-propanoate obtained in example 62 was hydrolyzed with aqueous LiOH at 25° C. for 12 h in methanol. THF mixture (3 mL+2 mL) after the completion of reaction the solvent was evaporated and the aqueous layer was washed once with ether and the aqueous layer was acidified with 2 N HCl to pH 2 and extracted with EtOAc and the organic layer was dried with Na₂SO₄ and evaporated under reduced pressure to give 2-{4-[3-(biphenyl-4-yloxy)-propyl]-phenoxy}-2-methyl-propanoic acid (83%).

M.P: 130-133° C.;

¹H NMR (3, CDCl₃, 200 MHz): 7.55-7.29 (m, 7H), 7.13 (d, J=8.60 Hz, 2H), 6.95 (d, J=8.59 Hz, 2H), 6.88 (d, J=8.60 Hz, 2H), 3.99 (t, J=6.18 Hz, 2H), 2.79 (t, J=7.30 Hz, 2H), 2.13-2.00 (m, 2H), 1.60 (s, 6H).

### EXAMPLE 64

### Ethyl 2-methyl-2-[3-{3-(4-methanesulfonyloxyphenoxy)propyloxy}phenoxy]propanoate

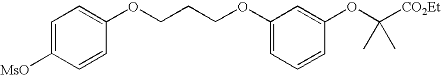

Obtained by following procedure of example 18 using starting materials obtained in preparation 11 and 13.

Yield: 357 mg, 49%

¹H NMR (CDCl₃, 400 MHz) δ: 1.23 (t, J=7.2 Hz, 3H); 1.59(s, 6H); 2.23 (quintet, 1=6 Hz, 2H); 3.10 (s, 3H); 4.10 (t, J=6 Hz, 2H); 4.13 (t, J=6 Hz, 2H); 4.23 (q, J=7.2 Hz, 2H); 6.38-6.41 (aromatics, 1H); 6.44-6.45 (aromatics, 1H); 6.53-6.56 (aromatics, 1H); 6.91 (d, J=9.2 Hz, 2H); 7.11 (t, J=8.4 Hz, 1H); 7.19 (d, J=9.2 Hz, 2H).

IR (neat) cm⁻¹: 2938, 1731, 1597, 1502.

Mass m/z (CI): 453 [M+1]

### EXAMPLE 65

### 2-Methyl-2-[3-{3-(4-methanesulfonyloxyphenoxy)propyloxy}phenoxy]propanoic acid

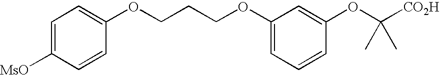

Obtained from example 64 by following procedure of example 23.

Yield: 120 mg, 36%.

¹H NMR (CDCl₃, 400 MHz) δ: 1.60 (s, 6H); 2.24 (quintet, J=6 Hz, 2H); 3.10 (s, 3H); 4.11 (t, J=6 Hz, 2H); 4.14 (t, J=6.2 Hz, 2H); 6.51-6.53 (aromatics, 2H); 6.62-6.65 (aromatics, 1H); 6.91 (d, J=9.1 Hz, 2H); 7.16-7.20 (aromatics, 3H)

IR (neat) cm⁻¹: 2937, 1717, 1596, 1502.

Mass m/z (ES): 425.1 [M+1], 442.3 [M+18], 866.5 [M₂+18].

### EXAMPLE 66

### 2-Methyl-2-[3-{3-(4-methanesulfonyloxyphenoxy)propyloxy}phenoxy]propanoic acid Arginine salt

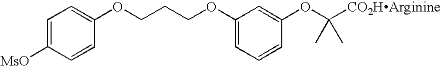

Obtained from example 65 by following procedure of example 45

Mp: 88-90° C.

Mass m/z (ES): 599.5 [M+1].

### EXAMPLE 67

### Ethyl 2-methyl-2-[3-{3-(5-methanesulfonyloxyindol-1-yl)propyl}phenoxy]propanoate

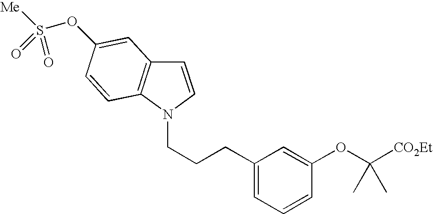

Obtained by following procedure of example 17 and using starting materials obtained in step-1 of preparation 7 and preparation 18.

Thick liquid. Yield: 600 mg (83%).

¹H NMR (CDCl₃, 400 MHz) δ: 1.22 (t, J=7.2 Hz, 3H); 1.59 (s, 6H); 2.15 (quintet, J=7.2 Hz, 2H); 2.58 (t, J=7.2 Hz, 2H); 3.12 (s, 3H); 4.11 (t, J=7.2 Hz, 2H); 4.21 (q, J=7.2 Hz, 2H); 6.51 (d, J=2.8 Hz, 1H); 6.66-6.70 (aromatics, 2H); 6.78 (d, J=7.6 Hz, 1H); 7.10-7.24 (aromatics, 4H); 7.53 (d, J=2.4 Hz, 1H).

IR (Neat, cm⁻¹): 2935, 1731, 1583, 1363.

Mass m/z (CI): 460 [M+1].

### EXAMPLE 68

### 2-methyl-2-[3-{3-(5-methanesulfonyloxyindol-1-yl)propyl}phenoxy]propanoic acid

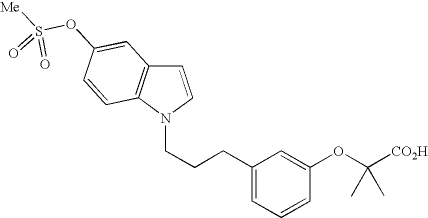

Obtained from example 67 by following procedure of example 23.

Thick liquid. Yield: 331 mg (67%).

¹H NMR (CDCl₃, 400 MHz) δ: 1.58 (s, 6H); 2.15 (quintet, J=7.2 Hz, 2H); 2.56 (t, J=7.6 Hz, 2H); 3.12 (s, 3H); 4.11 (t, J=7.2 Hz, 2H); 6.51 (d, J=3.2 Hz, 1H); 6.73 (s, 1H); 6.74-6.79 (aromatic, 1H); 6.86 (d, J=7.6 Hz, 1H); 7.10-7.24 (aromatics, 4H); 7.52 (d, J=2.4 Hz, 1H).

IR (Neat, cm⁻¹): 3362, 2937, 1717, 1362.

Mass m/z (ES): 432.3 [M+1], 449.4 [M+NH₄⁺], 453.3 [M+Na⁺], 880.5 [M₂+NH₄⁺].

### EXAMPLE 69

### 2-methyl-2-[3-{3-(5-methanesulfonyloxyindol-1-yl)propyl}phenoxy]propanoic acid Arginine salt

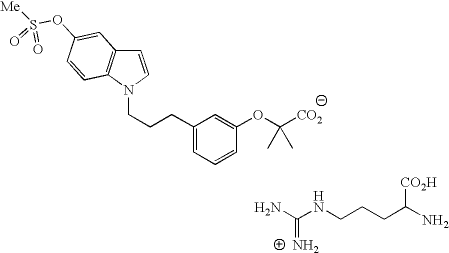

Obtained from example 68 by following procedure of example 45.

Mp: 85-87° C. (dec).

Mass m/z (ES): 606 [M+1].

### EXAMPLE 70

### Ethyl 2-methyl-2-[3-{3-(7-Methanesulfonyloxy-3,4-dihydro-2H-bezo[b][1,4]oxazin-4-yl)propyl}phenoxy]propanoate

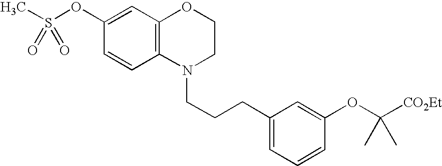

This compound was made using the typical procedure described for example 18 except that Na₂CO₃ was used as base instead of K₂CO₃, and also a mixture of MeCN/DMF was used as solvent instead of DMF alone. Starting materials were obtained from preparation 19 and 24. Yield: 170 mg, 10%.

¹H NMR (CDC₃, 400 MHz) δ: 1.24 (t, J=7.2 Hz, 3H); 1.59 (s, 6H); 1.80-1.91 (m, 2H); 2.61 (t, J=7.6 Hz, 2H); 3.07 (s, 3H); 3.22 (t, J=7.2 Hz, 2H); 3.29 (t, J=4.4 Hz, 2H); 4.19-4.26 (m, 4H); 6.46 (d, J=8.8 Hz, 1H); 6.65-6.68 (aromatic, 2H); 6.71-6.72 (aromatic, 2H); 6.81-6.84 (aromatic, 1H); 7.12-7.17 (aromatic, 1H).

IR (Neat, cm⁻¹): 3397, 2927, 1730, 1585.

Mass m/z (CI): 478 [M+1].

### EXAMPLE 71

### (+) Methyl (R)-2-methyl-2-[4-{3-(5-methanesulfonyloxyindol-1-yl)propyl}phenoxy]butanoate

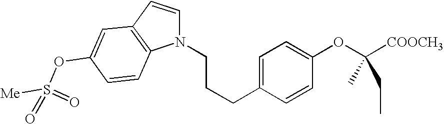

A solution of powdered KOH (203 mg, 1.6 eq, 3.62 mmol) in dry DMSO (8 mL) was stirred at RT for 10 min, then 5-methanesulfonyloxyindole (956 mg, 2 eq, 4.53 mmol), obtained in step 1 of preparation 7, was added portion wise at RT and stirring was continued at RT for 20 min. Then methyl (R)-2-methyl-2-[4-(3-methanesulfonyloxypropyl)phenoxy]butanoate (780 mg, 1 eq, 2.27 mmol), obtained in preparation 31, in 3 mL of dry DMSO was added drop wise at RT. And the reaction was stirred at RT for 2 h. Being guided by TLC, reaction was stopped. Reaction mixture was diluted with ethyl acetate (150 mL), and washed with water (2×100 mL). Organic layer was dried (Na₂SO₄), condensed, and the residue was chromatographed using ethyl acetate and hexane to obtain the title compound as thick mass. (875 mg, 88% yield).

¹H NMR (CDCl₃, 400 MHz) δ: 0.98 (t, J=7.6 Hz, 3H); 1.48 (s, 3H); 1.94-1.99 (m, 2H); 2.14 (quintet, J=7.2 Hz, 2H); 2.56 (t, J=7.6 Hz, 2H); 3.12 (s, 3H); 3.77 (s, 3H); 4.10 (t, J=6.8 Hz, 2H); 6.50 (d, J=3.2 Hz, 1H); 6.77 (d, J=8.4 Hz, 2H); 7.01 (d, J=8.4 Hz, 2H); 7.11 (dd, J=2.4, 8.8 Hz, 1H); 7.14 (d, J=3.2 Hz, 1H); 7.23 (d, J=8.8 Hz, 1H); 7.52 (d, J=2.4 Hz, 1H).

IR (neat) cm⁻¹: 2942, 1736, 1509, 1360.

Mass m/z (CI): 460 [M+1].

[α]_{D}=+13° (c=1%, MeOH, 25° C.).

### EXAMPLE 72

### (−) Methyl (S)-2-methyl-2-[4-{3-(5-methanesulfonyloxyindol-1-yl)propyl}phenoxy]butanoate

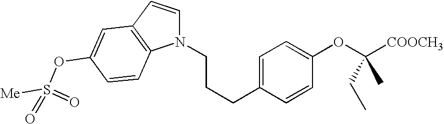

The title compound which is an enantiomer of example 71 was obtained following the procedure for example 71 and using starting material obtained in preparation 32.

[α]_{D}=−13.2° (c=1%, MeOH, 25° C.)

Using the typical procedure described in example 15 and 18 the following examples (examples 73-77) have been obtained.

### EXAMPLE 73

### Ethyl 2-methyl-2-[3-{3-(4-(para-toluenesulfonyloxy)phenoxy)propyloxy}phenoxy]propanoate

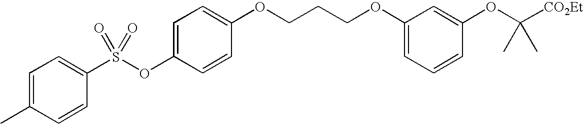

Yield: 825 mg, 89%.

¹H NMR (CDCl₃, 400 MHz) δ: 1.23 (t, J=7.1 Hz, 3H); 1.59 (s, 6H); 2.20 (quintet, J=6.1 Hz, 2H); 2.44 (s, 3H); 4.07 (t, J=6.1 Hz, 4H); 4.22 (q, J=7.1 Hz, 2H); 6.40 (ddd, J=8.1, 2.3, 0.6 Hz, 1H); 6.44 (t, J=2.3 Hz, 1H); 6.53 (ddd, J=8.1, 2.3, 0.6 Hz, 1H); 6.76 (d, J=9.1 Hz, 2H); 6.86 (d, J=9.1 Hz, 2H); 7.10 (t, J=8.1 Hz, 1H); 7.30 (d, J=8.1 Hz, 2H); 7.68 (d, J=8.1 Hz, 2H).

IR (neat) cm⁻¹: 2985, 1733, 1598, 1501, 1172.

Mass m/z (CI): 529 [M+1].

### EXAMPLE 74

### Ethyl 2-methyl-2-[4-{3-(4-methanesulfonyloxyphenoxy)propyloxy}phenoxy]butanoate

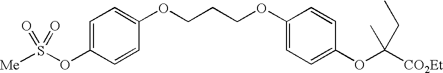

Obtained using starting materials from preparation 13 and 26.

Yield: 825 mg, 89%.

¹H NMR (CDCl₃, 400 MHz): 0.98 (t J=7.5 Hz, 3H); 1.27 (t, J=7.1 Hz, 3H); 1.41 (s, 3H); 1.88-2.00 (m, 2H); 2.20-2.26 (m, 2H); 3.09 (s, 3H); 4.09 (t, J=6 Hz, 2H); 4.14 (t, J=6 Hz, 2H); 4.24 (q, J=7.1 Hz, 2H); 6.77 (d, J=9.4 Hz, 2H); 6.83 (d, J=9.4 Hz, 2H); 6.90 (d, J=9.1 Hz, 2H); 7.19 (d, J=9.1 Hz, 2H).

IR (neat) cm⁻¹: 2977, 1731, 1504, 1196.

Mass m/z (CI): 467 [M+1].

### EXAMPLE 75

### Ethyl 2-methyl-2-[4-{4-(4-methanesulfonyloxyphenoxy)butyl}phenoxy]propanoate

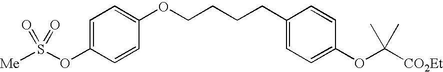

Obtained using starting materials from preparation 12 and 35.

Yield: 400 mg, 55%.

¹H NMR (CDCl₃, 400 MHz) δ: 1.25 (t, J=7.2 Hz, 3H); 1.57 (s, 6H); 1.75-1.82 (m, 4H); 2.61 (t, J=7.3 Hz, 2H); 3.10 (s, 3H); 3.94 (t, J=5.9 Hz, 2H); 4.26 (q, J=7.2 Hz, 2H); 6.77 (d, J=8.6 Hz, 2H); 6.88 (d, J=8.9 Hz, 2H); 7.05 (d, J=8.5 Hz, 2H); 7.18 (d, J=8.9 Hz, 2H).

IR (neat) cm⁻¹: 2938, 1729, 1593, 1502, 1149.

Mass m/z(CI): 451 [M+1].

### EXAMPLE 76

### Ethyl 2-methyl-2-[3-{5-(4-methanesulfonyloxyphenoxy)pentyl}phenoxy]propanoate

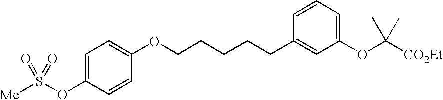

Obtained using starting materials from preparation 12 and 36.

Yield: 625 mg, 42%.

¹H NMR (CDCl₃, 400 MHz) δ: 1.23 (t, J=7.2 Hz, 3H); 1.42-1.55 (m, 2H); 1.58 (s, 6H); 1.60-1.75 (m, 2H); 1.75-1.85 (m, 2H); 2.58 (t, J=7.6 Hz, 2H); 3.1 (s, 3H); 3.92 (t, J=6.5 Hz, 2H); 4.22 (q, J=7.2 Hz, 2H); 6.64 (dd, J=8.1, 2.4 Hz, 1H); 6.70 (s, 1H); 6.80 (d, J=7.5 Hz, 1H); 6.87 (d, J=9.2 Hz, 2H); 7.13 (t, J=7.6 Hz, 1H); 7.18 (d, J=9.2 Hz, 2H).

IR (neat) cm⁻¹: 2938, 1732, 1602, 1502, 1151.

Mass m/z(ES): 464 [M], 465 [M+1]

### EXAMPLE 77

### Ethyl 2-methyl-2-[3-{5-(4-nitrophenoxy)propyl}phenoxy]propanoate

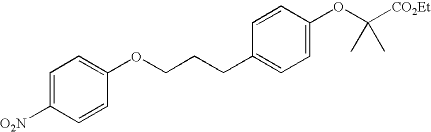

Obtained using starting material from preparation 16 and reacting with 4-nitrophenol.

Yield: 170 mg, 75%.

¹H NMR (CDCl₃, 200 MHz) δ: 1.25 (t, J=7.2 Hz, 3H); 1.57 (s, 6H); 2.08-2.12 (m, 2H); 2.75 (t, J=7.4 Hz, 2H); 4.02 (t, J=6.2 Hz, 2H); 4.23 (q, J=7.2 Hz, 2H); 6.77 (d, J=8.4 Hz, 2H); 6.92 (d, J=9.1 Hz, 2H); 7.05 (d, J=8.4 Hz, 2H); 8.19 (d, J=9.1 Hz, 2H).

IR (neat) cm⁻¹: 2937, 1733, 1519, 1262.

Mass m/z(CI): 388 [M+1].

### EXAMPLE 78

### Ethyl 2-methyl-2-[3-{5-(4-aminophenoxy)propyl}phenoxy]propanoate

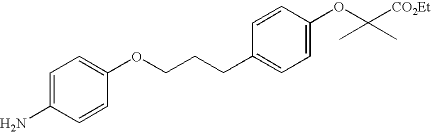

Obtained using starting materials from example 77 and doing hydrogenation (10% Pd/C, H₂ (1 atm)) in ethyl acetate solvent at RT.

Yield: 825 mg, 97%.

¹H NMR (CDCl₃, 400 MHz) δ: 1.24 (t, J=7.2 Hz, 3H); 1.56 (s, 6H); 2.0-2.08 (m, 2H); 2.71 (t, J=7.6 Hz, 2H); 3.86 (t, J=6.4 Hz, 2H); 4.22 (q, J=7.2 Hz, 2H); 6.62 (d, J=8.8 Hz, 2H); 6.72 (d, J=8.8 Hz, 2H); 6.76 (d, J=8.7 Hz, 2H); 7.05 (d, J=8.7 Hz, 2H).

IR (neat) cm⁻¹: 3366, 2938, 1731, 1510, 1233, 1140.

Mass m/z(CI): 358 [M+1].

### EXAMPLE 79

### Ethyl 2-methyl-2-[4-{3-(4-(tert-butyloxycarbonylamino)phenoxy)propyl}phenoxy]propanoate

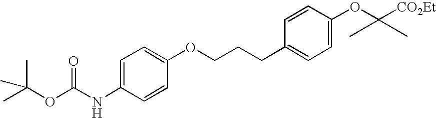

Obtained using starting materials from example 78 and reacting with (BOC)₂O in presence of triethylamine in dichloromethane solvent at 0-RT for 3 h.

Yield: 143 mg, 28%.

¹H NMR (CDCl₃, 400 MHz) δ: 1.24 (t, J=7.2 Hz, 3H); 1.38 (s, 2.25H, minor rotamer); 1.44 (s, 6.75H, major rotamer); 1.57 (s, 6H); 2.0-2.08 (m, 2H); 2.72 (t, J=6.4 Hz, 2H); 3.93 (t, J=6.4 Hz, 2H); 4.23 (q, J=7.2 Hz, 2H); 6.77 (d, J=8.6 Hz, 2H); 6.83 (d, J=9.0 Hz, 1.5H, major rotamer); 6.87 (d, J=9.0 Hz, 0.5H, minor rotamer); 7.05 (d, J=8.6 Hz, 2H); 7.06 (bs, NH, 1H); 7.14 (d, J=9.0 Hz, 1.5H, major rotamer); 7.44 (d, J=9.0 Hz, 0.5H, minor rotamer).

IR (neat) cm⁻¹: 3381, 2934, 1734, 1509, 1241, 1145.

Mass m/z(CI): 457 [M], 458 [M+1].

### EXAMPLE 80

### Ethyl 2-methyl-2-[4-{3-(4-(methanesulfonylamino)phenoxy)propyl}phenoxy]propanoate

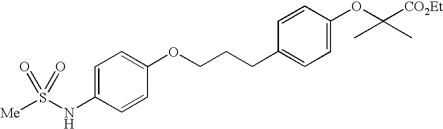

Obtained using starting material from example 78 and reacting with methanesulfonyl chloride in presence of triethylamine in dichloromethane solvent at 0-RT for 4 h.

Yield: 400 mg, 79%.

¹H NMR (CDCl₃, 400 MHz) δ: 1.25 (t, J=7 Hz, 3H); 1.57 (s, 6H); 2.00-2.08 (m, 2H); 2.72 (t, J=7.5 Hz, 2H); 3.38 (s, 3H); 3.95 (t, J=6.2 Hz, 2H); 4.23 (q, J=7 Hz, 2H); 6.78 (d, J=8.6 Hz, 2H); 6.91 (d, J=8.8 Hz, 2H); 7.05 (d, J=8.6 Hz, 2H); 7.24 (d, J=8.8 Hz, 2H).

IR (neat) cm⁻¹: 3383, 2926, 1732, 1367, 1162.

Mass m/z(CI): 436 [M+1].

Using the typical procedure described in example 17 and 71 the following examples (examples 81-86) have been obtained.

### EXAMPLE 81

### Ethyl 2-methyl-2-[4-{4-(5-methanesulfonyloxyindol-1-yl)butyl}phenoxy]propanoate

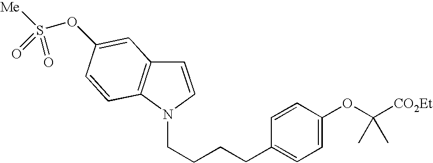

Obtained using starting materials from step-1 of preparation 7 and preparation 35.

Yield: 640 mg, 57%.

¹H NMR (CDCl₃, 400 MHz) δ: 1.24 (t, J=7.3 Hz, 3H); 1.56 (s, 6H); 1.57-1.65 (m, 2H); 1.80-1.90 (m, 2H); 2.55 (t, J=7.5 Hz, 2H); 3.12 (s, 3H); 4.11 (t, J=6.8 Hz, 2H); 4.22 (q, J=7.3 Hz, 2H); 6.49 (dd, J=3.2, 0.6 Hz, 1H); 6.75 (d, J=8.6 Hz, 2H); 6.97 (d, J=8.6 Hz, 2H); 7.10-7.15 (aromatics, 2H); 7.28 (d, J=8.8 Hz, 1H); 7.51 (d, J=2.2 Hz, 1H).

IR (neat) cm⁻¹: 2937, 1732, 1177.

Mass m/z (CI): 474 [M+1].

### EXAMPLE 82

### Ethyl 2-methyl-2-[3-{3-(5-para-toluenesulfonyloxy)indol-1-yl)propyl}phenoxy]propanoate

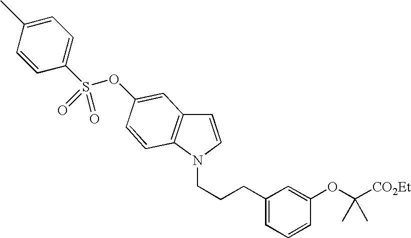

Obtained using starting materials from preparation 18 and 34.

Yield: 110 mg, 12%.

¹H NMR (CDCl₃, 400 MHz) δ: 1.24 (t, J=7.2 Hz, 3H); 1.61 (s, 6H); 2.14 (quintet, J=7.3 Hz, 2H); 2.46 (s, 3H); 2.58 (t, J=7.3 Hz, 2H); 4.09 (t, J=7.3 Hz, 2H); 4.23 (q, J=7.2 Hz, 2H); 6.42 (d, J=9, 2.9 Hz, 1H); 6.68-6.78 (aromatics, 2H); 6.79-6.84 (aromatics, 2H); 7.12-7.32 (aromatics, 6H); 7.74 (d, J=8.4 Hz, 2H).

IR (neat) cm⁻¹: 2985, 2929, 1732, 1599, 1178.

Mass m/z (CI): 536 [M+1].

### EXAMPLE 83

### Ethyl 2-[3-{3-(5-methanesulfonyloxyindol-1-yl)propyl}phenoxy]propanoate

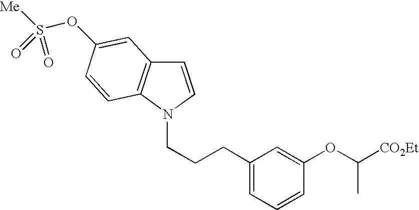

Obtained using starting material from step-1 of preparation 7 and preparation 37.

Yield: 350 mg, 60%.

¹H NMR (CDCl₃, 400 MHz) δ: 1.22 (t, J=7.2 Hz, 3H); 1.60 (d, J=6.7 Hz, 3H); 2.10-2.20 (m, 2H); 2.59 (t, J=7.5 Hz, 2H); 3.12 (s, 3H); 4.11 (t, J=7.0 Hz, 2H); 4.20 (q, J=7.2 Hz, 2H); 4.72 (q, J=6.7 Hz, 1H); 6.51 (d, J=0.6 Hz, 1H); 6.70-9-6.82 (aromatics, 3H); 7.10-7.25 (aromatics, 4H); 7.52 (d, J=2.2 Hz, 1H).

IR (neat) cm⁻¹: 2936, 1747, 1175.

Mass m/z (CI): 446 [M+1].

### EXAMPLE 84

### 1-[4-{3-(5-Methanesulfonyloxyindol-1-yl)propyl}phenoxy]cyclohexane-1-carboxylic acid, methyl ester

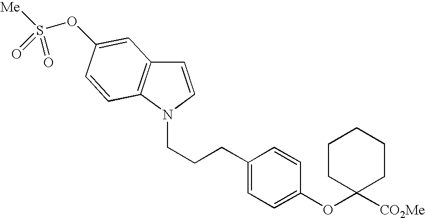

Obtained using starting material from step-1 of preparation 7 and preparation 38.

Yield: 190 mg, 36%.

¹H NMR (CDCl₃, 400 MHz) δ: 1.50-1.75 (m, 6H); 1.82-1.92 (m, 2H); 2.05-2.20 (m, 4H); 2.55 (t, J=7.6 Hz, 2H); 3.11 (s, 3H); 3.75 (s, 3H); 4.10 (t, J=7.4 Hz, 2H); 6.50 (d, J=2.8 Hz, 1H); 6.75 (d, J=8.6 Hz, 2H); 7.01 (d, J=8.6 Hz, 2H); 7.09-7.17 (aromatics, 2H); 7.22 (d, J=8.8 Hz, 1H); 7.52 (d, J=2.2 Hz, 1H).

IR (neat) cm⁻¹: 2938, 2859, 1733, 1508, 1364, 1224, 1178.

Mass m/z (CI): 486 [M+1].

### EXAMPLE 85

### 1-[4-{3-(5-methanesulfonyloxyindol-1-yl)propyl}phenoxy]cyclopentane-1-carboxylic acid, methyl ester

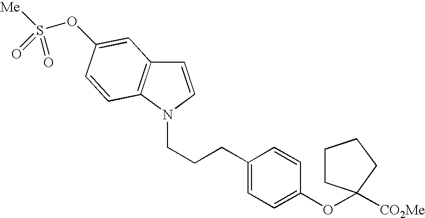

Obtained using starting material from step-1 of preparation 7 and preparation 39.

Yield: 780 mg, 92%.

¹H NMR (CDCl₃, 400 MHz) δ: 1.72-1.86 (m, 4H); 2.10-2.21 (m, 4H); 2.21-2.30 (m, 2H); 2.55 (t, J=7.6 Hz, 2H); 3.11 (s, 3H); 3.73 (s, 3H); 4.10 (t, J=7 Hz, 2H); 6.50 (d, J=3 Hz, 1H); 6.68 (d, J=8.8 Hz, 2H); 7.01 (d, J=8.8 Hz, 2H); 7.09-7.10 (aromatics, 2H); 7.23 (d, J=8.8 Hz, 1H); 7.52 (d, J=2.4 Hz, 1H).

IR (neat) cm⁻¹: 2931, 1712, 1508, 1362, 1225, 1176.

Mass m/z (CI): 472 [M+1].

### EXAMPLE 86

### 1-[4-{4-(4-methanesulfonyloxyindol-1-yl)butyl}phenoxy]cyclopentane-1-carboxylic acid, methyl ester

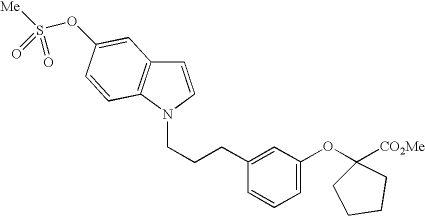

Obtained using starting material from step-1 of preparation 7 and preparation 40.

Yield: 600 mg, 83%.

¹H NMR (CDCl₃, 400 MHz) δ: 1.57-1.63 (m, 2H); 1.70-1.88 (m, 6H); 2.10-2.20 (m, 2H); 2.20-2.30 (m, 2H); 2.54 (t, J=7.7 Hz, 2H); 3.12 (s, 3H); 3.72 (s, 3H); 4.11 (t, J=7.2 Hz, 2H); 6.48 (d, J=3.2 Hz, 1H); 6.65 (d, J=8.6 Hz, 2H); 6.97 (d, J=8.6 Hz, 2H); 7.10-7.14 (aromatics, 2H); 7.28 (d, J=9.0 Hz, 1H); 7.52 (d, J=2.5 Hz, 1H).

IR (neat) cm⁻¹: 2939, 1734, 1611, 1508, 1177.

Mass m/z (ES): 486 [M+1], 503.4 [M+NH₄⁺], 508.3 [M+Na⁺], 988.7 [M₂+NH₄⁺], 993.5 [M₂+Na⁺].

### EXAMPLE 87

### 1-[4-{3-(7-Methanesulfonyloxy-3,4-dihydro-2H-bezo[b][1,4]oxazin-4-yl)propyl}phenoxy]cyclopentane-1-carboxylic acid, methyl ester

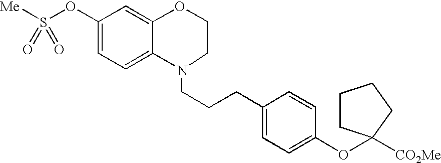

Using the typical procedure described in example 70 the title compound has been obtained.

Yield: 170 mg, 10%.

¹H NMR (CDCl₃, 400 MHz) δ: 1.75-1.90 (m, 6H); 2.10-2.20 (m, 2H); 2.20-2.30 (m, 2H); 2.58 (t, J=7.5 Hz, 2H); 3.07 (s, 3H); 3.22 (t, J=7.4 Hz, 2H); 3.28 (t, J=4.4 Hz, 2H); 3.73 (s, 3H); 4.20 (t, J=4.4 Hz, 2H); 6.46 (d, J=6.2 Hz, 1H); 6.67 (s, 1H); 6.69 (d, J=6 Hz, 1H); 6.75 (d, J=8.6 Hz, 2H); 7.04 (d, J=8.6 Hz, 2H);

IR (neat) cm⁻¹: 3418, 2947, 1735, 1509, 1236, 1179.

Mass m/z (CI): 489 [M], 490 [M+1].

### EXAMPLE 88

### Ethyl 2-methyl-2-[4-{4-(7-methanesulfonyloxy-3,4-dihydro-2H-bezo[b][1,4]oxazin-3 on-4-yl)butyl}phenoxy]propanoate

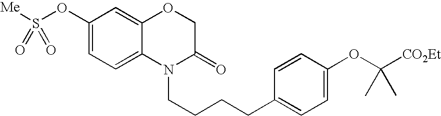

Using the typical procedure described in example 70 the title compound has been obtained.

Yield: 330 mg, 52%.

¹H NMR (CDCl₃, 400 MHz) δ: 1.25 (t, J=7.2 Hz, 3H); 1.57 (s, 6H); 1.55-1.70 (m, 4H); 2.60 (t, J=6.6 Hz, 2H); 3.15 (s, 3H); 3.91 (t, J=7 Hz, 2H); 4.23 (q, J=7.2 Hz, 2H); 4.61 (s, 2H); 6.77 (d, J=8.8 Hz, 2H); 6.85-6.98 (aromatics, 3H); 7.02 (d, J=8.8 Hz, 2H).

IR (neat) cm⁻¹: 2936, 1732, 1687, 1506.

Mass m/z (CI): 506 [M+1].

Using the general ester hydrolysis procedure described in example 23 the following examples (examples 89-105) were obtained from their corresponding esters.

### EXAMPLE 89

### 2-Methyl-2-[3-{3-(7-Methanesulfonyloxy-3,4-dihydro-2H-bezo[b][1,4]oxazin-4-yl)propyl}phenoxy]propanoic acid

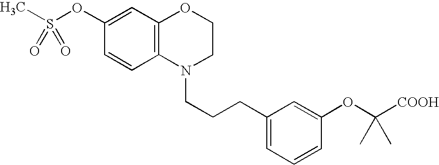

Yield: 100 mg, 63%.

¹H NMR (CDCl₃, 400 MHz) δ: 1.60 (s, 6H); 1.86-1.95 (m, 2H); 2.63 (t, J=7.4 Hz, 2H); 3.08 (s, 3H); 3.24 (t, J=7.4 Hz, 2H); 3.29 (t, J=4.4 Hz, 2H); 4.21(t, J=4.4 Hz, 2H); 6.44 (d, J=8.8 Hz, 2H); 6.47-6.75 (aromatic, 2H); 6.77-6.79 (aromatic, 2H); 6.91 (d, J=7.6 Hz, 1H); 7.18-7.23 (aromatic, 1H).

IR (Neat, cm⁻¹): 3380, 2935, 1730, 1602, 1511.

Mass m/z (ES): 450 [M+1], 472.1 [M+Na], 921.7 [M₂+Na].

### EXAMPLE 90

### (R)-(+)-2-Methyl-2-[4-{3-(5-methanesulfonyloxyindol-1-yl)propyl}phenoxy]butanoic acid

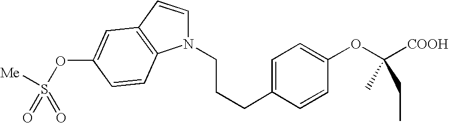

The hydrolysis was done by following the typical procedure described for example 23 except that the solvent mixture was MeOH-water and the reaction time was 3 days.

Thick liquid. Yield: 730 mg (88%)

¹H NMR (CDCl₃, 400 MHz) δ: 1.04 (t, J=7.6 Hz, 3H); 1.47 (s, 3H); 1.90-1.99 (m, 1H); 1.99-2.03 (m, 1H); 2.14 (quintet, J=7.6 Hz, 2H); 2.59 (t, J=7.8 Hz, 2H); 3.12 (s, 3H); 4.12 (t, J=6.9 Hz, 2H); 6.50 (d, J=3.2 Hz, 1H); 6.88 (d, J=8.8 Hz, 2H); 7.05 (d, J=8.4 Hz, 2H); 7.11 (dd, J=2.4, 8.8 Hz, 1H); 7.15 (d, J=3.2 Hz, 1H); 7.22 (d, J=8.8 Hz, 1H); 7.52 (d, J=2.4 Hz, 1H).

IR (neat) cm⁻¹: 2940, 1716, 1509.

Mass m/z (ES): 446.3 [M+1], 463.4 [M+NH₄], 468.5 [M+Na], 913.7 [M₂+Na]

[α]_{D}=+10° (c=1%, MeOH, 25° C.)

### EXAMPLE 91

### (S)-(−)-2-methyl-2-[4-{3-(5-methanesulfonyloxyindol-1-yl)propyl}phenoxy]butanoic acid

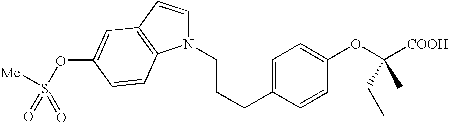

[α]_{D}=−10° (c=1%, MeOH, 25° C.)

### EXAMPLE 92

### 2-methyl-2-[3-{3-(para-toluenesulfonyloxy)phenoxy)propyloxy}phenoxy]propanoic acid

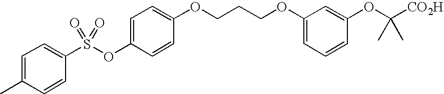

Yield: 190 mg, 66%.

¹H NMR (CDCl₃, 400 MHz) δ: 1.59 (s, 6H); 2.21 (quintet, J=6.1 Hz, 2H); 2.44 (s, 3H); 4.07-4.11 (m, 4H); 6.50-6.53 (aromatics, 2H); 6.62 (d, J=8.6 Hz, 1H); 6.76 (d, J=9.2 Hz, 2H); 6.87 (d, J=9.2 Hz, 2H); 7.15 (t, J=8.6 Hz, 1H); 7.29 (d, J=8.1 Hz, 2H); 7.69 (d, J=8.1 Hz, 2H).

IR (neat) cm⁻¹: 3500, 2926, 1713, 1597, 1501, 1150.

Mass m/z (ES): 501.3 [M+1], 518.5 [M+NH₄⁺], 523.3 [M₂+NH₄⁺]

### EXAMPLE 93

### 2-Methyl-2-[4-{3-(4-methanesulfonyloxyphenoxy)propyloxy}phenoxy]butanoic acid

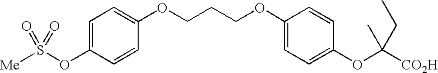

Yield: 70 mg, 13%.

¹H NMR (CDCl₃, 400 MHz) δ: 1.05 (t, J=7.4 Hz, 3H); 1.41 (s, 3H); 1.82-2.00 (m, 2H); 2.22-2.28 (m, 2H); 3.11 (s, 3H); 4.15 (t, J=6 Hz, 2H); 4.12 (t, J=6 Hz, 2H); 6.82 (d, J=9 Hz, 2H); 6.89-6.94 (aromatics, 4H); 7.19 (d, J=9.1 Hz, 2H).

IR (neat) cm⁻¹: 3360, 2926, 1723, 1593, 1503.

Mass m/z (ES): 456 [M+NH₄⁺], 894.5 [M₂+NH₄⁺].

### EXAMPLE 94

### 2-Methyl-2-[4-{4-(4-methanesulfonyloxyphenoxy)butyl}phenoxy]propanoic acid

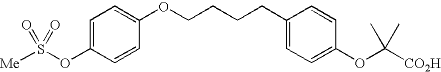

Yield: 110 mg, 59%.

¹H NMR (CDCl₃, 400 MHz) δ: 1.56 (s, 6H); 1.15-1.84 (m, 4H); 2.65 (t, J=7.2 Hz, 2H); 3.1 (s, 3H); 3.95 (t, J=5.9 Hz, 2H); 6.87 (d, J=9.4 Hz, 4H); 7.10 (d, J=8.6 Hz, 2H); 7.18 (d, J=9.2 Hz, 2H).

IR (neat) cm⁻¹: 3441, 2938, 1716, 1503, 1151.

Mass m/z(ES): 440 [M+NH₄⁺], 445 [M+Na⁺], 867.5 [M₂+Na⁺].

### EXAMPLE 95

### 2-Methyl-2-[3-{5-(4-methanesulfonyloxyphenoxy)pentyl}phenoxy]propanoic acid

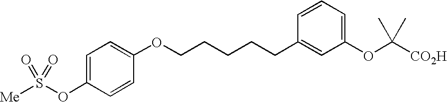

Yield: 180 mg, 64%.

¹H NMR (CDCl₃, 400 MH [symbol] □: 1.42-1.55 (m, 2H); 1.59 (s, 6H); 1.60-1.75 (m, 2H); 1.75-1.85 (m, 2H); 2.60 (t, J=7.6 Hz, 2H); 3.1 (s, 3H); 3.93 (t, J=6.4 Hz, 2H); 6.70-6.78 (aromatics, 2H); 6.87-6.91 (aromatics, 1H); 6.88 (d, J=9.1 Hz, 2H); 7.17-7.21 (aromatics, 1H); 7.19 (d, J=9.1 Hz, 2H).

IR (neat) cm⁻¹: 3342, 2936, 1716, 1502, 1151.

Mass m/z(ES): 454.3 [M+NH₄⁺], 459.3 [M+Na⁺], 890.5[M₂+NH₄⁺], 895.5 [M₂+Na⁺].

### EXAMPLE 96

### 2-Methyl-2-[4-{3-(4-(tert-butyloxycarbonylamino)phenoxy)propyl}phenoxy]propanoic acid

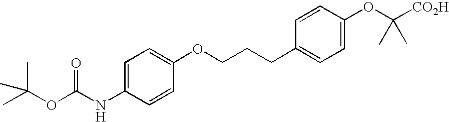

Hydrolysis was done using K₂CO₃ as base instead of LiOH.

Yield: 35 mg, 27%.

Mp: 132-134° C.

¹H NMR (CDCl₃, 400 MHz) δ: 1.51 (s, 9H); 1.57 (s, 6H); 2.00-2.08 (m, 2H); 2.75 (t, J=7.4 Hz, 2H); 3.90 (t, J=6.2 Hz, 2H); 6.4 (bs, NH, 1H); 6.78 (d, J=8.8 Hz, 2H); 6.85 (d, J=8.5 Hz, 2H); 7.10 (d, J=8.5 Hz, 2H); 7.22 (d, J=8.8 Hz, 2H).

IR (neat) cm⁻¹: 3307, 2931, 1703, 1506, 1159.

Mass m/z(ES): 430 [M+1], 447.4 [M+NH₄⁺], 452.3 [M+Na⁺], 876.8 [M₂+NH₄⁺].

### EXAMPLE 97

### 2-Methyl-2-[4-{3-(4-(methanesulfonylamino)phenoxy)propyl}phenoxy]propanoic acid

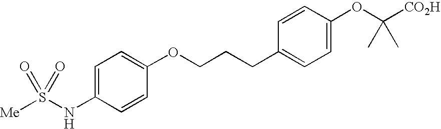

Yield: 50 mg, 12%.

Mp: 122-124° C.

¹H NMR (CDCl₃, 400 MH [symbol] □: 1.57 (s, 6H); 2.00-2.08 (m, 2H); 2.76 (t, J=7.6 Hz, 2H); 2.95 (s, 3H); 3.93 (t, J=6.3 Hz, 2H); 6.2 (bs, NH, 1H); 6.86 (apparent triplet, J=8.6 Hz, 4H); 7.11 (d, J=8.8 Hz, 2H); 7.16 (d, J=8.8 Hz, 2H).

IR (neat) cm⁻¹: 3441, 2926, 1729, 1510, 1147.

Mass m/z(ES): 425 [M+NH₄⁺], 430 [M+Na⁺], 837 [M₂+Na⁺].

### EXAMPLE 98

### 2-Methyl-2-[4-{4-(5-methanesulfonyloxyindol-1yl)butyl}phenoxy]propanoic acid

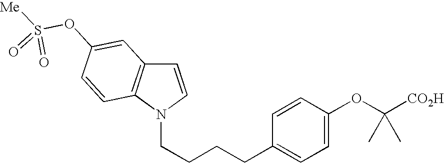

Yield: 110 mg, 49%.

¹H NMR (CDCl₃, 400 MHz) δ: 1.57 (s, 6H); 1.57-1.65 (m, 2H); 1.80-1.90 (m, 2H); 2.57 (t, J=7.6 Hz, 2H); 3.12 (s, 3H); 4.11 (t, J=7.3 Hz, 2H); 6.49 (dd, J=3.2, 0.6 Hz, 1H); 6.85 (d, J=8.4 Hz, 2H); 7.00 (d, J=8.4 Hz, 2H); 7.09-7.13 (aromatics, 2H); 7.27 (d, J=9.5 Hz, 1H); 7.51 (d, J=2.1 Hz, 1H).

IR (neat) cm⁻¹: 3375, 2936, 1715, 1177.

Mass m/z (ES): 446.1 [M+1], 463.3 [M+NH₄⁺], 468.4 [M+Na⁺].

### EXAMPLE 99

### 2-Methyl-2-[3-{3-(5-para-toluenesulfonyloxy)indol-1-yl)propyl}phenoxy]propanoic acid

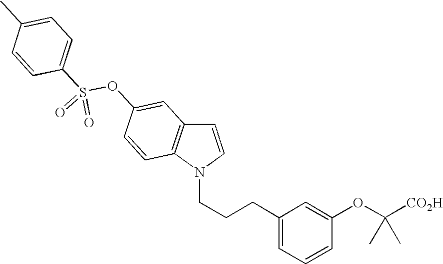

Yield: 49 mg, 47%.

¹H NMR (CDCl₃, 400 MHz) δ: 1.58 (s, 6H); 2.12-2.17 (m, 2H); 2.44 (s, 3H); 2.57 (t, J=7.5 Hz, 2H); 4.08 (t, J=7.0 Hz, 2H); 6.41 (d, J=2.9 Hz, 1H); 6.73-6.87 (aromatics, 4H); 7.07-7.17 (aromatics, 2H); 7.18-7.22 (aromatics, 2H); 7.29 (d, J=8.3 Hz, 2H); 7.73 (d, J=8.3 Hz, 2H).

IR (neat) cm⁻¹: 3383, 2932, 1733, 1674, 1600, 1178.

Mass m/z (ES): 508 [M+1], 525 [M+NH₄⁺], 530 [M+Na⁺].

### EXAMPLE 100

### 2-[3-{3-(5-Methanesulfonyloxyindol-1-yl)propyl}phenoxy]propanoic acid

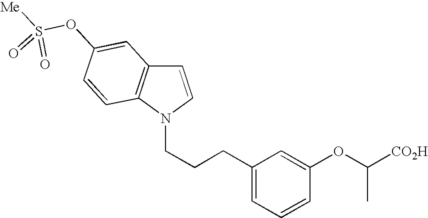

Yield: 230 mg, 70%.

¹H NMR (CDCl₃, 400 MHz) δ: 1.63 (d, J=6.9 Hz, 3H); 2.15 (quintet, J=7.2 Hz, 2H); 2.57 (t, J=7.5 Hz, 2H); 3.12 (s, 3H); 4.11 (t, J=7.0 Hz, 2H); 4.75 (q, J=6.9 Hz, 1H); 6.50 (d, J=0.6 Hz, 1H); 6.68 (s, 1H); 6.72 (d, J=2.2 Hz, 1H); 6.74 (d, J=1.9 Hz, 1H); 7.10-7.22 (aromatics, 4H); 7.51 (d, J=2.4 Hz, 1H).

IR (neat) cm⁻¹: 3378, 2936, 1725, 1177.

Mass m/z (ES): 418 [M+1], 435 [M+NH₄⁺], 440.3 [M+Na⁺], 857.5 [M₂+Na⁺].

### EXAMPLE 101

### 1-[4-{3-(5-methanesulfonyloxyindol-1-yl)propyl}phenoxy]cyclohexane-1-carboxylic acid

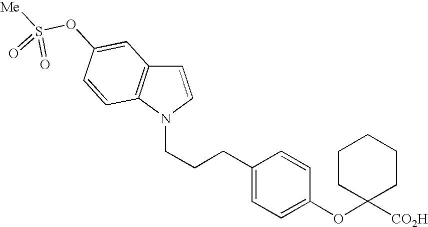

Yield: 36 mg, 21%.

¹H NMR (CDCl₃, 400 MHz) δ: 1.50-1.65 (m, 6H); 1.80-1.95 (m, 2H); 2.11-2.20 (m, 4H); 2.57 (t, J=7.6 Hz, 2H); 3.12 (s, 3H); 4.10 (t, J=7 Hz, 2H); 6.50 (d, J=3.3 Hz, 1H); 6.83 (d, J=8.5 Hz, 2H); 7.02 (d, J=8.5 Hz, 2H); 7.09-7.17 (aromatics, 2H); 7.20 (d, J=8.8 Hz, 1H); 7.50 (d, J=2.1 Hz, 1H).

IR (neat) cm⁻¹: 3500, 2938, 1733, 1509, 1386, 1224, 1178.

Mass m/z (ES): 472.1 [M+1], 489.1 [M+NH₄⁺], 494.5 [M+Na⁺], 960.5 [M₂+NH₄⁺].

### EXAMPLE 102

### 1-[4-{3-(5-Methanesulfonyloxyindol-1-yl)propyl}phenoxy]cyclopentane-1-carboxylic acid

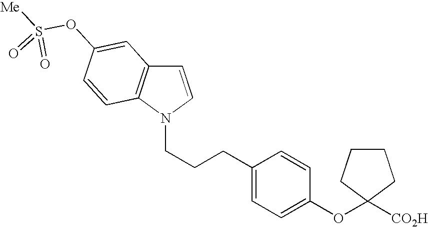

Yield: 230 mg, 30%.

¹H NMR (CDCl₃, 400 MHz) δ: 1.75-1.86 (m, 4H); 2.10-2.22 (m, 4H); 2.22-2.35 (m, 2H); 2.55 (t, J=7.5 Hz, 2H); 3.11 (s, 3H); 4.10 (t, J=7.2 Hz, 2H); 6.50 (d, J=3.2 Hz, 1H); 6.74 (d, J=8.6 Hz, 2H); 7.01 (d, J=8.6 Hz, 2H); 7.09-7.10 (aromatics, 2H); 7.21 (d, J=8.8 Hz, 1H); 7.51 (d, J=2.4 Hz, 1H).

IR (neat) cm⁻¹: 3400, 2937, 1710, 1510, 1363, 1177.

Mass m/z (ES): 458 [M+1], 475.4 [M+NH₄⁺], 480.1 [M+Na⁺], 932.5 [M₂+NH₄⁺], 937.3 [M₂+Na⁺].

### EXAMPLE 103

### 1-[4-{4-(5-methanesulfonyloxyindol-1-yl)butyl}phenoxy]cyclopentane-1-carboxylic acid

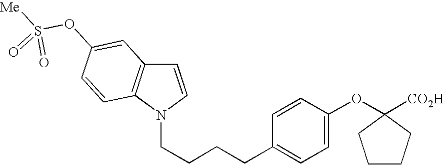

Yield: 240 mg, 42%.

¹H NMR (CDCl₃, 400 MHz) δ: 1.55-1.63 (m, 2H); 1.70-1.88 (m, 6H); 2.15-2.22 (m, 2H); 2.22-2.35 (m, 2H); 2.55 (t, J=7.5 Hz, 2H); 3.12 (s, 3H); 4.10 (t, J=7.1 Hz, 2H); 6.48 (d, J=3.3 Hz, 1H); 6.71 (d, J=8.4 Hz, 2H); 6.98 (d, J=8.4 Hz, 2H); 7.09-7.13 (aromatics, 2H); 7.26 (d, J=9.0 Hz, 1H); 7.50 (d, J=2.5 Hz, 1H).

IR (neat) cm⁻¹: 3375, 2926, 1730, 1609, 1508, 1177.

Mass m/z (ES): 472 [M+1], 489 [M+NH₄⁺], 494.3 [M+Na⁺], 960.3 [M₂+NH₄⁺], 965.2 [M₂+Na⁺].

### EXAMPLE 104

### 1-[4-{3-(7-Methanesulfonyloxy-3,4-dihydro-2H-bezo[b][1,4]oxazin-4-yl)propyl}phenoxy]cyclopentane-1-carboxylic acid

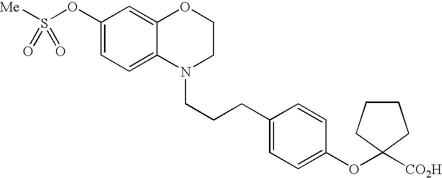

Yield: 20 mg, 23%.

¹H NMR (CDCl₃, 400 MHz) δ: 1.75-1.90 (m, 6H); 2.15-2.25 (m, 2H); 2.25-2.35 (m, 2H); 2.59 (t, J=7.4 Hz, 2H); 3.07 (s, 3H); 3.21 (t, J=7.5 Hz, 2H); 3.28 (t, J=4.4 Hz, 2H); 4.20 (t, J=4.4 Hz, 2H); 6.38 (d, J=6.2 Hz, 1H); 6.68 (s, 1H); 6.69 (d, J=6 Hz, 1H); 6.76 (d, J=8.6 Hz, 2H); 7.06 (d, J=8.6 Hz, 2H);

IR (neat) cm⁻¹:

Mass m/z (CI): 476 [M+1].

### EXAMPLE 105

### 2-Methyl-2-[4-{4-(7-methanesulfonyloxy-3,4-dihydro-2H-bezo[b][1,4]oxazin-3-on-4-yl)butyl}phenoxy]propanoic acid

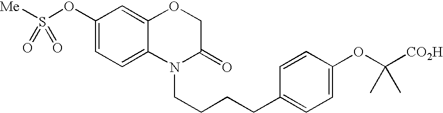

Yield: 120 mg, 40%.

¹H NMR (CDCl₃, 400 MHz) δ: 1.58 (s, 6H); 1.62-1.68 (m, 4H); 2.62 (t, J=6.8 Hz, 2H); 3.17 (s, 3H); 3.91 (t, J=7 Hz, 2H); 4.62 (s, 2H); 6.84-6.90 (aromatics, 5H); 7.07 (d, J=9.2 Hz, 2H).

IR (neat) cm⁻¹: 3383, 2934, 1730, 1682, 1505, 1123.

Mass m/z (ES): 478 [M+1], 495.3 [M+NH₄⁺], 472.2 [M₂+NH₄⁺].

The following arginine salts (examples 106-117) were obtained from their corresponding acids following the procedure described in example 45.

### EXAMPLE 106

### 2-Methyl-2-[3-{3-(7-Methanesulfonyloxy-3,4-dihydro-2H-bezo[b][1,4]oxazin-4-yl)propyl}phenoxy]propanoic acid, Arginine salt

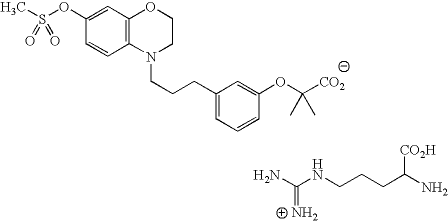

Mp: 130-132° C.

### EXAMPLE 107

### (R)-(+)-2-methyl-2-[4-{3-(5-methanesulfonyloxyindol-1-yl)propyl)phenoxy]butanoic acid, Arginine salt

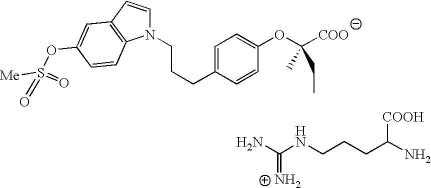

Mp: 100-102° C. (dec).

### EXAMPLE 108

### (S)-(−)-2-methyl-2-[4-{3-(5-methanesulfonyloxyindol-1-yl)propyl}phenoxy]butanoic acid, Arginine salt

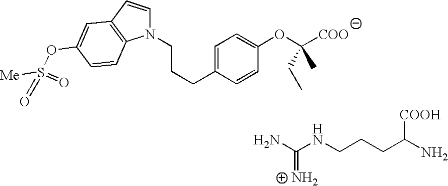

This Arginine salt was made using the typical procedure described for example 45.

Mp: 110° C. (dec),

### EXAMPLE 109

### 2-Methyl-2-[3-{3-(4-para-toluenesulfonyloxy)phenoxy)propyloxy}phenoxy]propanoic acid, arginine salt

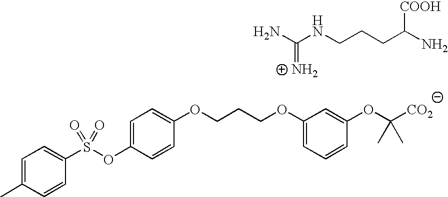

Mp: 118-120° C.

### EXAMPLE 110

### 2-Methyl-2-[4-{3-(4-methanesulfonyloxyphenoxy)propyloxy}phenoxy]butanoic acid, arginine salt

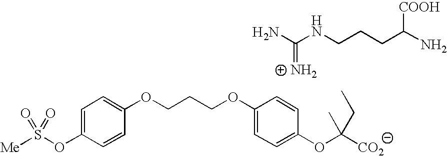

Mp: 90° C. (dec).

### EXAMPLE 111

### 2-Methyl-2-[4-{4-(4-methanesulfonyloxyphenoxy)butyl}phenoxy]propanoic acid, arginine salt

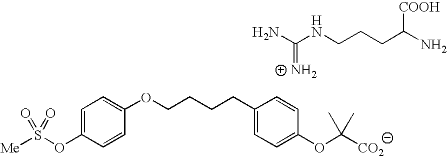

### EXAMPLE 112

### 2-Methyl-2-[3-{5-(4-methanesulfonyloxyphenoxy)pentyl}phenoxy]propanoic acid, arginine salt

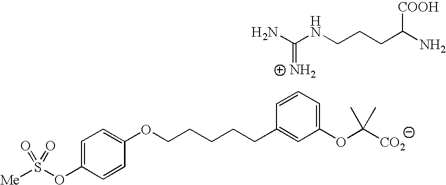

Mp: 106-108° C.

### EXAMPLE 113

### 2-Methyl-2-[4-{4-(5-methanesulfonyloxyindol-1yl)butyl}phenoxy]propanoic acid, arginine salt

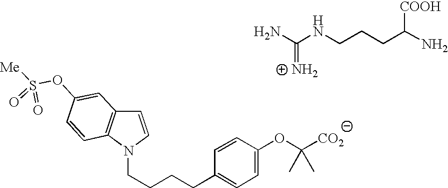

Mp: 128-130° C.

### EXAMPLE 114

### 2-Methyl-2-[3-{3-(5-(para-toluenesulfonyloxy)indol-1-yl)propyl}phenoxy]propanoic acid, arginine salt

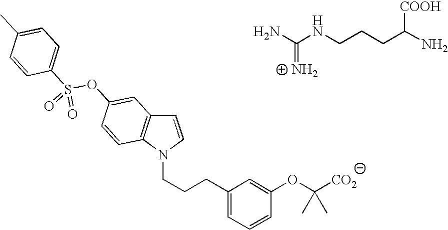

Mp: 118° C.

### EXAMPLE 115

### 2-[3-{3-(5-Methanesulfonyloxyindol-1-yl)propyl}phenoxy]propanoic acid, arginine salt

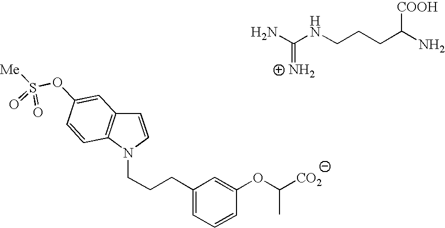

Arg salt: Mp: 110 112° C.

### EXAMPLE 116

### 1-[4-{4-(5-Methanesulfonyloxyindol-1-yl)butyl}phenoxy]cyclopentane-1-carboxylic acid, arginine salt

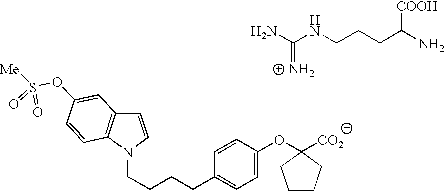

Mp: 98-100° C.

### EXAMPLE 117

### 2-Methyl-2-[4-(4-(7-methanesulfonyloxy-3,4-dihydro-2H-bezo[b][1,4]oxazin-3-on-4-yl)butyl}phenoxy]propanoic acid, Arginine salt

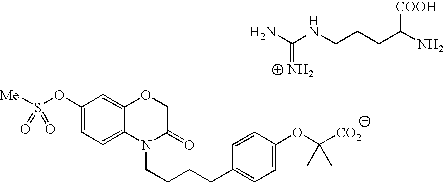

Mp: 126-128° C.

### EXAMPLE 118

### Methyl-2-methyl-2-[4-{3-(5-methanesulfonyloxyindol-1-yl)propyl}phenoxy]butanoic acid Magnesium salt

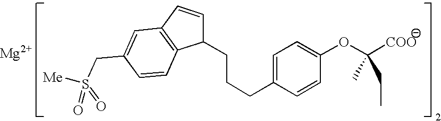

A solution of racemic 2-methyl-2-[4-{3-(5-methanesulfonyloxyindol-yl)propyl}phenoxy]butanoic acid (1 mmol), obtained following a racemic synthesis of example 90, in 5 mL of dry MeOH was treated with 0.5 mmol of Mg(OMe)₂ and the reaction mixture was stirred at 70° C. for 17 h. Then MeOH was completely removed and it was azeotropically dried with benzene. Finally it was dried over high vacuum pump to get the salt as almost white solid (quantitative yield). Mp: 136° C. (dec).

### EXAMPLE 119

### 1-[4-{3-(5-methanesulfonyloxyindol-1-yl)propyl}phenoxy]cyclohexane-1-carboxylic acid, magnesium salt

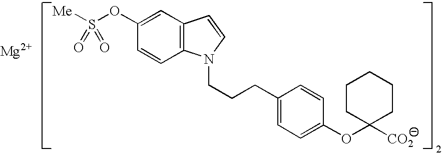

Mp: 138° C. (dec).

### EXAMPLE 120

### 1-[4-{3-(5-Methanesulfonyloxyindol-1-yl)propyl}phenoxy]cyclopentane-1-carboxylic acid, magnesium salt

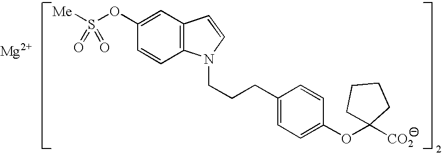

Mp: 111° C. (dec).

### EXAMPLE 121

### 1-[4-{3-(7-Methanesulfonyloxy-3,4-dihydro-2H-bezo[b][1,4]oxazin-4-yl)propyl}phenoxy]cyclopentane-1-carboxylic acid, magnesium salt

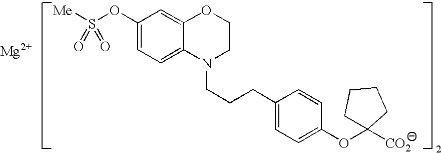

Mg salt: Mp: 158-160° C. (dec).

Demonstration of Efficacy of Compounds

The compounds of the present invention lower random blood sugar level, triglyceride, total cholesterol, LDL, VLDL and increase HDL and insulin sensitivity. This may be demonstrated by in vitro as well as in vivo animal experiments.

In Vitro:

a) Determination of hPPARα Activity

Ligand binding domain of hPPARα was fused to A binding domain of Yeast transcription factor GAL4 in eucaryotic expression vector. Using superfect (Qiagen, Germany) as transfecting reagent HEK-293 cells are transfected with this plasmid and a reporter plasmid harboring the luciferase gene driven by a GAL4 specific promoter. Compound can be added at different concentrations after 42 hrs of transfection and incubated overnight. Luciferase activity as a function of compound binding/activation capacity of PPARα will be measured using Packard Luclite kit (Packard, USA) in Top Count (Ivan Sadowski, Brendan Bell, Peter Broag and Melvyn Hollis. Gene. 1992. 118 : 137-141; Superfect Transfection Reagent Handbook. February 1997. Qiagen, Germany).

b) Determination of hPPARγ Activity

Ligand binding domain of hPPARγ1 is fused to DNA binding domain of Yeast transcription factor GAL4 in eucaryotic expression vector. Using lipofectamine (Gibco BRL, USA) as transfecting reagent HEK-293 cells are transfected with this plasmid and a reporter plasmid harboring the luciferase gene driven by a GAL4 specific promoter. Compound can be added at 1 μM concentration after 48 hrs of transfection and incubated overnight Luciferase activity as a function of drug binding/activation capacity of PPARγ1 will be measured using Packard Luclite kit (Packard, USA) in Packard Top Count (Ivan Sadowski, Brendan Bell, Peter Broag and Melvyn Hollis. Gene. 1992. 118 : 137-141; Guide to Eukaryotic Transfections with Cationic Lipid Reagents. Life Technologies, GIBCO BRL, USA).

PPAR fold activation

| | PPAR α at 50 μM | PPAR γ at 1 μM |
|---|---|---|
| Compound | concentration | concentration |
| Example 1 | 2.7 | 9.2 |
| Example 2 | 3.5 | 15.5 |
| Example 24 | 7.4 | 4.5 |
| Example 45 | 5.2 | 1.4 |
| Example 46 | 3.2 | 2.1 |
| Example 47 | 4.8 | 6.7 |
| Example 48 | 4.6 | 6.1 |
| Example 49 | 3.6 | 1.5 |
| Example 50 | 3.5 | 1.4 |
| Example 52 | 4.0 | 6 |
| Example 53 | 3.7 | 5.8 |
| Example 55 | 3.8 | 11.9 |

In Vivo

a) Efficacy in Genetic Models

Mutation in colonies of laboratory animals and different sensitivities to dietary regimens have made the development of animal models with non-insulin dependent diabetes and hyperlipidemia associated with obesity and insulin resistance possible. Genetic models such as db/db and ob/ob (Diabetes, (1982) 31(1) : 1-6) mice and zucker fa/fa rats have been developed by the various laboratories for understanding the pathophysiology of disease and testing the efficacy of new antidiabetic compounds (Diabetes, (1983) 32: 830-838; Annu. Rep. Sankyo Res. Lab. (1994). 46: 1-57). The homozygous animals, C57 BL/KsJ-db/db mice developed by Jackson Laboratory, US, are obese, hyperglycemic, hyperinsulinemic and insulin resistant (J. Clin. Invest., (1990) 85 : 962-967), whereas heterozygous are lean and normoglycemic. In db/db model, mouse progressively develops insulinopenia with age, a feature commonly observed in late stages of human type II diabetes when blood sugar levels are insufficiently controlled. The state of pancreas and its course vary according to the models. Since this model resembles that of type II diabetes mellitus, the compounds of the present invention will be tested for blood sugar and triglycerides lowering activities.

Male C57BL/KsJ-db/db mice of 8 to 14 weeks age, having body weight range of 35 to 60 grams, bred at Dr. Reddy's Research Foundation (DRF) animal house, were used in the experiment. The mice are provided with standard feed (National Institute of Nutrition (NIN), Hyderabad, India) and acidified water, ad libitum. The animals having more than 350 mg/dl blood sugar will be used for testing. The number of animals in each group will be 4.

Test compounds are suspended on 0.25% carboxymethyl cellulose and administered to test group at a dose of 0.1 mg to 30 mg/kg through oral gavage daily for 6 days. The control group receives vehicle (dose 10 ml/kg). On 6th day the blood samples will be collected one hour after administration of test compounds/vehicle for assessing the biological activity.

The random blood sugar and triglyceride levels can be measured by collecting blood (100 μl) through orbital sinus, using heparinised capillary in tubes containing EDTA which was centrifuged to obtain plasma. The plasma glucose and triglyceride levels can be measured spectrometrically, by glucose oxidase and glycerol-3-PO₄ oxidase/peroxidase enzyme (Dr. Reddy's Lab. Diagnostic Division Kits, Hyderabad, India) methods respectively.

| | db/db | | |
|---|---|---|---|
| | | % of reduction in | % of reduction in |
| Compound | Dose mg/kg/d | Plasma glucose | Triglyceride |
| Example 45 | 3 | 32 | 28 |

b) Plasma Triglyceride and Total Cholesterol Lowering Activity in Swiss Albino Mice and Guinea Pigs

Male Swiss albino mice (SAM) and male Guinea pigs are obtained from NIN and housed in DRF animal house. All these animals are maintained under 12 hour light and dark cycle at 25±1° C. Animals are given standard laboratory chow (NIN, Hyderabad, India) and water, ad libitum. SAM of 20-25 g body weight range and Guinea pigs of 500-700 g body weight range are used (Oliver, P., Plancke, M. O., Marzin, D., Clavey, V., Sauzieres, J and Fruchart, J. C. Effects of fenofibrate, gemfibrozil and nicotinic acid on plasma lipoprotein levels in normal and hyperlipidemic mice. Atherosclerosis. 1988. 70 : 107-114).

The test compounds can be administered orally to Swiss albino mice at 0.3 to 30 mg/kg/day dose for 6 days. Control mice are treated with vehicle (0.25% Carboxymethylcellulose; dose 10 ml/kg). The test compounds are administered orally to Guinea pigs at 0.3 to 30 mg/kg/day dose for 6 days. Control animals are treated with vehicle (0.25% Carboxymethylcellulose; dose 5 ml/kg).

The blood samples can be collected in fed state 1 hour after drug administration on 0 and 6 day of treatment. The blood can be collected from the retro-orbital sinus through heparinised capillary in EDTA containing tubes. After centrifugation, plasma sample was separated for triglyceride and total cholesterol (Wieland, O. Methods of Enzymatic analysis. Bergermeyer, H. O., Ed., 1963. 211-214; Trinder, P. Ann. Clin. Biochem. 1969. 6 : 24-27). Measurement of plasma triglyceride, total cholesterol and HDL are done using commercial kits (Dr. Reddy's Diagnostic Division, Hyderabad, India).

Swiss albino mice

| | | % of |
|---|---|---|
| | Dose | Reduction in |
| Compound | (mg/kg/d) | Triglyceride |
| Example 2 | 3 | 50 |
| Example 24 | 3 | 46 |
| Example 26 | 3 | 10 |
| Example 37 | 3 | 30 |
| Example 45 | 3 | 60 |
| Example 46 | 10 | 12 |
| Example 47 | 3 | 60 |
| Example 48 | 3 | 27 |
| Example 49 | 3 | 54 |
| Example 50 | 3 | 31 |
| Example 53 | 3 | 79 |
| Example 54 | 3 | 43 |
| Example 55 | 3 | 60 |
| Example 61 | 3 | 51 |
| Example 66 | 3 | 71 |

1-27. (canceled)

28. A compound of the formula (I), 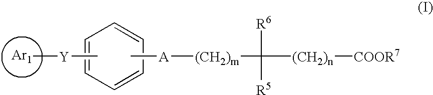
their derivatives, their stereoisomers, their pharmaceutically acceptable salts and their pharmaceutically acceptable compositions;
wherein Ar₁ represents a unsubstituted or substituted monocyclic or polycyclic aromatic or partially saturated aromatic polycyclic structure, which may optionally contain up to 3 heteroatoms selected from N, S or O, such as 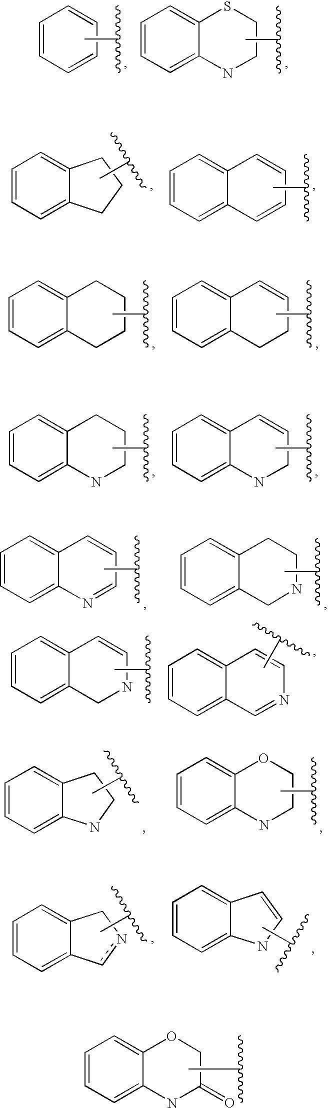
which when substituted may have up to 4 substituents that may be identical or different, wherein said substituents selected from halo, nitro, alkyl, hydroxy, hydroxyalkyl, alkoxy, thioalkoxy, oxo, aryl, —NR¹R², —OCONR¹R², NR¹COOR², —NR¹COR², —NR¹SO₂R², NR¹CONR¹R², —OSO₂R³, —SO₂R³,
R¹ and R² independently represent hydrogen, or optionally substituted groups selected from alkyl, alkenyl, alkynyl, cylcoalkyl, heterocyclyl, aryl, heteroaryl; R³ independently represents hydrogen, or optionally substituted groups selected from alkyl, alkenyl, alkynyl, cylcoalkyl, heterocyclyl, aryl, heteroaryl, wherein said substitutents on R¹, R² and R³ are selected from hydrogen, halo, nitro, amino, mono or di substituted amino, hydroxy, alkoxy, carboxy, cyano, alkyl, cycloalkyl, alkoxy, haloalkoxy, haloalkyl, cycloalkyl, aryl, heterocyclyl, heteroaryl;
m and n independently represents an integer from 0 to 6;
A represents O, S or a bond;
Y is selected from (CH₂)ₚ, (CH₂)ₚB(CH₂)_{q}, (CH₂)ᵣB(CH₂)ₚD(CH₂)_{q}, wherein p, q and r each independently represents an integer from 0 to 6; B and D independently represents S, O, NR⁴ or a bond, R⁴ represents hydrogen, alkyl, alkenyl, —S(O)₂—R⁸ or —C(O)R⁸, R⁸ is alkyl, alkoxy; with the proviso that when B and D represents a hetero atom p is not zero;
R⁵ and R⁶ independently represents hydrogen, alkyl, cycloalkyl or alkoxy; R⁵ and R⁶ together may form 3-8 membered cyclic ring which may optionally contains one or two hetero atoms selected from O, S or N;
R⁷ represents hydrogen, substituted or unsubstituted alkyl, cycloalkyl, alkenyl or alkynyl; wherein said substitutents are selected from hydrogen, halo, nitro, amino, mono or di substituted amino, hydroxy, alkoxy, carboxy, cyano, alkyl, cycloalkyl, alkoxy, haloalkoxy, haloalkyl, cycloalkyl, aryl, heterocyclyl, heteroaryl.

29. A compound of formula (Ia) according to claimed 28 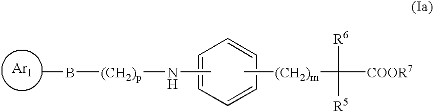
wherein all of the symbols are as defined above.

30. The compound of claim 29, wherein Ar₁ is substituted with —OSO₂R³, and R³ is alkyl or aryl.

31. The compound of formula (Ia) as claimed in claim 28 is selected from

| Structure | IUPAC Name |
|---|---|
| 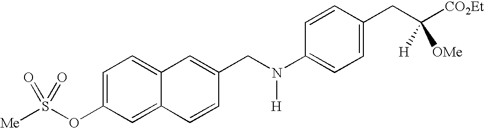 | (S)-Ethyl 2-methoxy-3- [4-{6-methanesulfonyloxynapth-2- ylmethylamino} phenyl] propanoate |
| 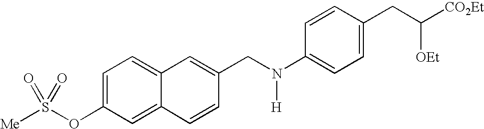 | Ethyl 2-ethoxy-3- [4-{6-methanesulfonyloxynapth-2- ylmethylamino} phenyl] propanoate |
| 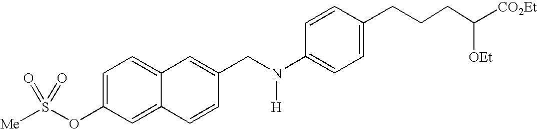 | Ethyl 2-ethoxy-5- [4-{6-methanesulfonyloxynapth-2- ylmethylamino} phenyl] pentanoate |
| 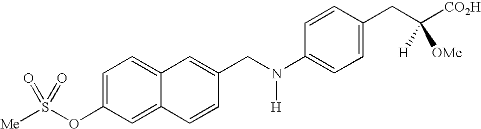 | (S)-2-methoxy-3- [4-{6-methanesulfonyloxynapth-2- ylmethylamino} phenyl] propanoic acid |
| 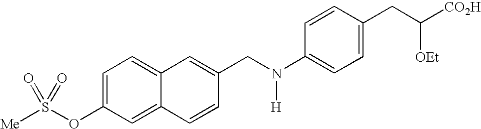 | 2-ethoxy-3- [4-{6-methanesulfonyloxynapth-2- ylmethylamino} phenyl] propanoic acid |
| 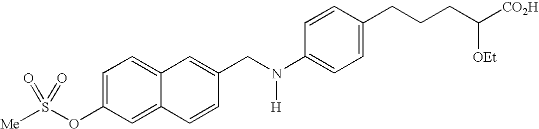 | 2-Ethoxy-5- [4-{6-methanesulfonyloxynapth-2- ylmethylamino} phenyl] pentatonic acid |

32. The compound of formula (Ia) as claimed in claim 28 is selected from

| Structure | IUPAC Name |
|---|---|
| 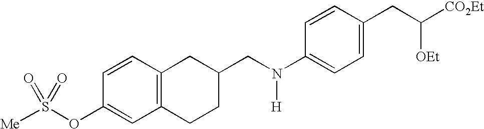 | Ethyl 2-ethoxy-3- [4-{(6-methanesulfonyloxy-1, 2, 3, 4- tetrahydronapth-2-yl) methylamino} phenyl] propanoate |
| 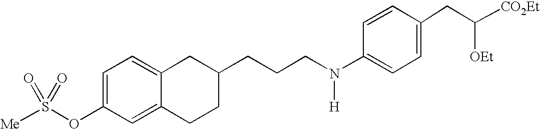 | Ethyl 2-ethoxy-3- [4-{3-(6-methanesulfonyloxy-1, 2, 3, 4- tetrahydronapth-2-yl) propylamino} phenyl] propanoate |
| 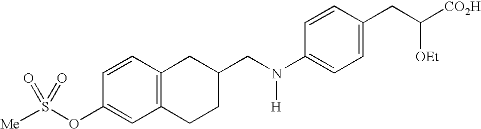 | 2-ethoxy-3- [4-{(6-methanesulfonyloxy-1, 2, 3, 4- tetrahydronapth-2-yl) methylamino} phenyl] propanoic acid |
| 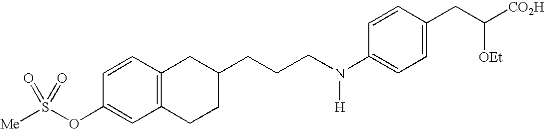 | 2-ethoxy-3- [4-{3-(6-methanesulfonyloxy-1, 2, 3, 4- tetrahydronapth-2-yl) propylamino} phenyl] propanoic aci |
| 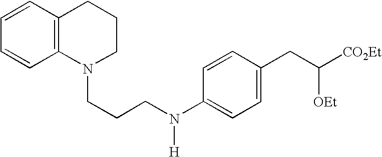 | Ethyl 2-ethoxy-3- [4-{3-(1,2,3,4-tetrahydroquinolyn-1-yl) propylamino} phenyl] propanoate |
| 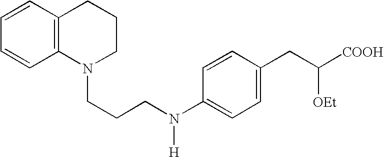 | 2-ethoxy-3- [4-{3-(1, 2, 3, 4-tetrahydroquinolyn-1-yl) propylamino} phenyl] propanoic acid |

33. The compound of formula (Ia) as claimed in claim 28 is selected from

| Structure | IUPAC Name |
|---|---|
| 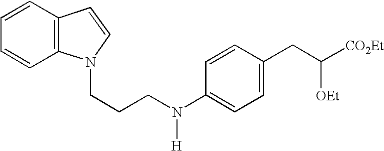 | Ethyl 2-ethoxy-3- [4-{3-(indol-1-yl) propyl amino} phenyl] propanoate |
| 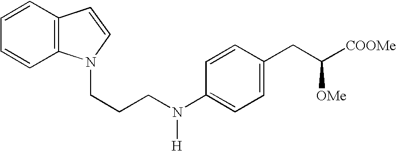 | (S)-Methyl 2-methoxy-3- [4-{3-(indol-1-yl) propylamino } phenyl] propanoate |
| 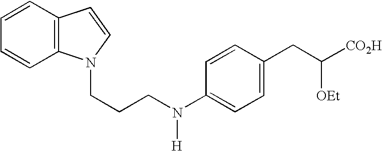 | 2-ethoxy-3- [4-{3-(indol-1-yl) propyl amino} phenyl] propanoic acid |
| 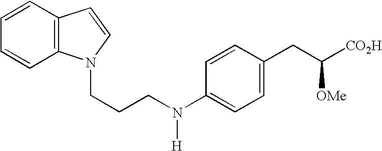 | (S)-2-methoxy-3- [4- {3-(indol-1-yl) propyl amino} phenyl] propanoic acid |
| 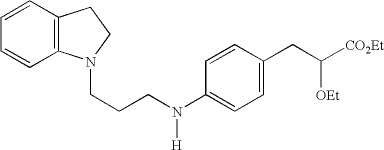 | Ethyl 2-ethoxy-3- [4-{3-(2, 3-dihydroindol-1-yl) propylamino} phenyl] propanoate |
| 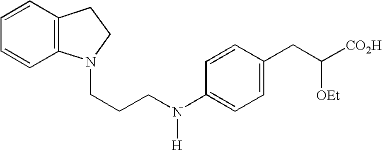 | 2-ethoxy-3- [4-{3-(2, 3-dihydroindol-1-yl) propylamino} phenyl] propanoic acid |

34. The compound of formula (Ia) as claimed in claim 28 is selected from

| Structure | IUPAC Name |
|---|---|
| 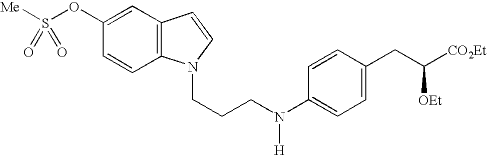 | (S)-Ethyl-2-ethoxy-3- [4-{3-(5-methanesulfonyloxyindol-1-yl) propylamino} phenyl] propanoate |
| 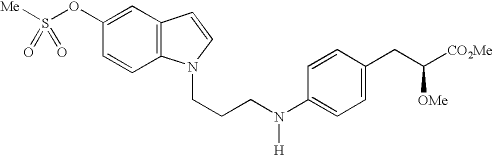 | S)-Methyl-2-methoxy-3- [4-{3-(5-methanesulfonyloxyindol-1- yl) propylamino} phenyl] propanoate |
| 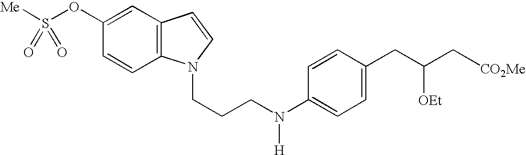 | (S)-Methyl 3-ethoxy-4- [4-{3-(5-methanesulfonyloxyindol-1- yl) propylamino} phenyl] butanoate |
| 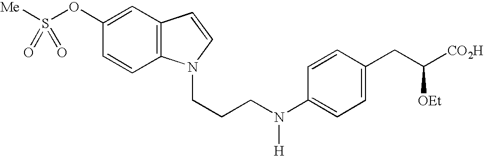 | (S)-2-ethoxy-3- [4-{3-(5-methanesulfonyloxyindol-1-yl) propylamino} phenyl] propanoic acid |
| 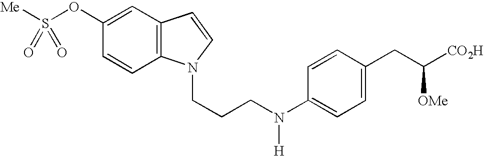 | S)-2-methoxy-3- [4-{3-(5-methanesulfonyloxyindol-1-yl) propylamino} phenyl] propanoic acid |
| 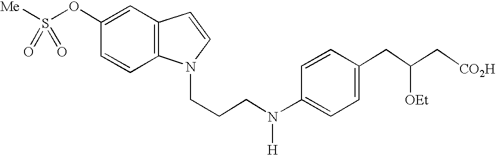 | S)-3-ethoxy-4- [4-{3 -(5-methanesulfonyloxyindol-1-yl) propylamino} phenyl] butanoic acid |

35. The compound of formula (Ia) as claimed in claim 28 is selected from

| Structure | IUPAC Name |
|---|---|
| 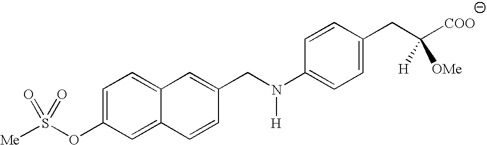 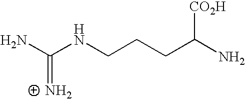 | (S)-2-methoxy-3- [4-{6-methanesulfonyloxynapth-2- ylmethylamino} phenyl] propanoic acid Arginine salt |
| 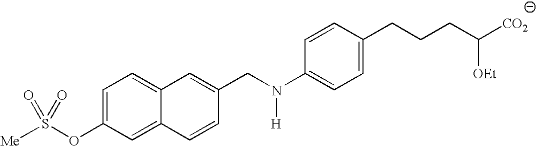 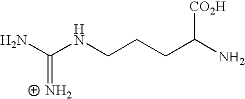 | 2-Ethoxy-5- [4-{6-methanesulfonyloxynapth-2- ylmethylamino} phenyl] pentatonic acid Arginine salt |
| 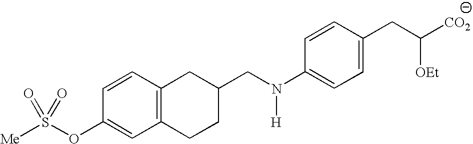 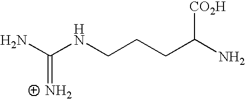 | 2-ethoxy-3- [4-{(6-methanesulfonyloxy-1, 2, 3, 4- tetrahydronapth-2-yl) methylamino} phenyl] propanoic acid Arginine salt |
| 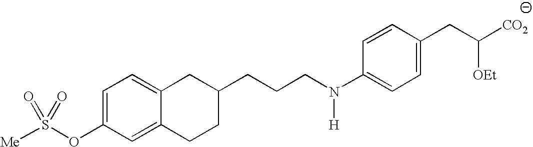 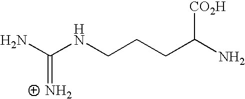 | 2-ethoxy-3- [4-{3-(6-methanesulfonyloxy-1, 2,3,4- tetrahydronapth-2-yl) propylamino} phenyl] propanoic acid Arginine salt |
| 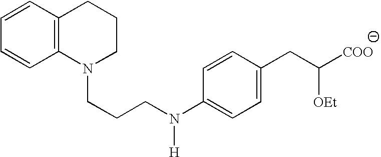 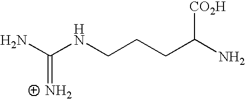 | 2-ethoxy-3- [4-{3-(1, 2, 3, 4-tetrahydroquinolyn-1-yl) propylamino} phenyl] propanoic acid Arginine salt |

36. The compound of formula (Ia) as claimed in claim 28 is selected from

| Structure | IUPAC Name |
|---|---|
| 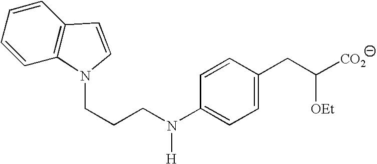 | 2-ethoxy-3-[4-{3-(indol-1-yl) propyl amino}phenyl]propanoic acid Arginine salt |
| 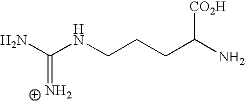 | |
| 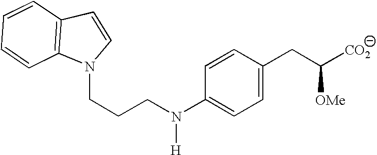 | (S)-2-methoxy-3-[4-{3-(indol-1-yl) propyl amino}phenyl]propanoic acid Arginine salt |
| 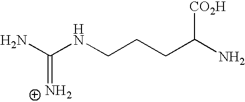 | |
| 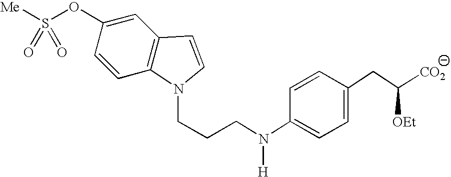 | (S)-2-ethoxy-3-[4-{3-(5- methanesulfonyl oxyindol-1-yl) propylamino}phenyl]propanoic acid Argrnine salt |
| 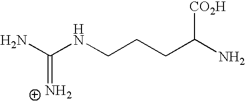 | |
| 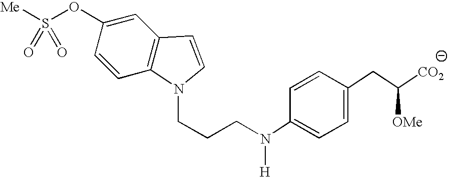 | (S)-2-methoxy-3-[4-{3-(5- methanesulfonyl oxyindol-1-yl) propylamino}phenyl]propanoic acid Arginine salt |
| 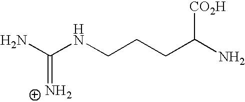 | |
| 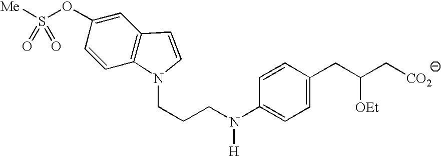 | (S)-3-ethoxy-4-[4-{3-(5- methanesulfonyloxyindol-1-yl) propylamino}phenyl]butanoic acid Arginine salt |
| 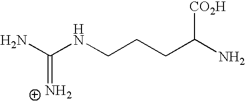 | |
| 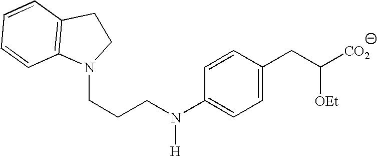 | 2-ethoxy-3-[4-{3-(2,3- dihydroindol-1-yl)propylamino}phenyl]propanoic acid Arginine salt |
| 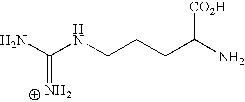 | |

37. The compound of formula (Ib) according to claimed 28, 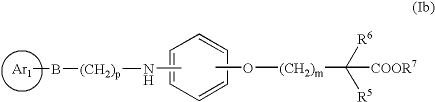
wherein all of the symbols are as defined above.

38. The compound of claim 37 wherein Ar₁ is substituted with —OSO₂R³, and R³ is alkyl or aryl.

39. The compound of formula (Ib) as claimed in claim 37 is selected from,

| Structure | IUPAC Name |
|---|---|
| 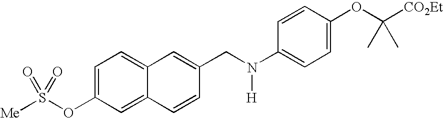 | Ethyl 2-methyl-2-[4-{6- methanesulfonyloxynapth-2- ylmethylamino}phenoxyl]propanoate |
| 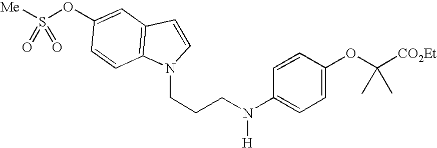 | Ethyl 2-methyl-2-[4-{3-(5- methanesulfonyloxyindol-1-yl) propylamino}phenoxy]propanoate |
| 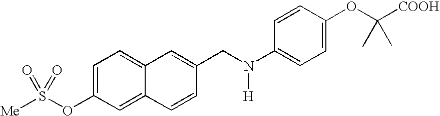 | 2-methyl-2-[4-{6- methanesulfonyloxynapth-2- ylmethylamino}phenoxy]propanoic acid |
| 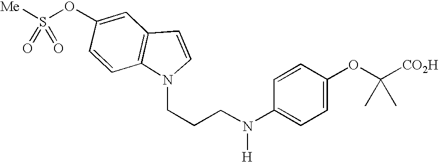 | 2-methyl-2-[4-{3-(5- methanesulfonyloxyindol-1-yl) propylamino}phenoxy]propanoic acid |

40. The compound of formula (Ic) according to claim 28 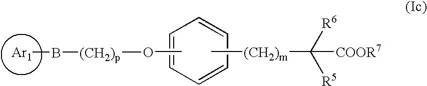
wherein all of the symbols are as defined above.

41. The compound of claim 40, wherein Ar₁ is substituted with —OSO₂R³, wherein R³ is alkyl or aryl.

42. The compound of formula (Id) according to claim 28, 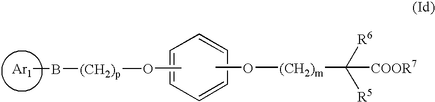
wherein all of the symbols are as defined above.

43. The compound of claim 42, wherein “Ar₁” is substituted with —OSO₂R³, where R³ is alkyl or aryl.

44. A compound of formula (Id) according to claim 28 which is selected from:

| Structure | IUPAC Name |
|---|---|
| 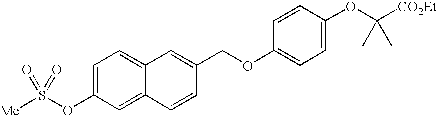 | Ethyl 2-methyl-2-[4-{6- methanesulfonyloxynapth-2- ylmethoxy}phenoxy]propanoate |
| 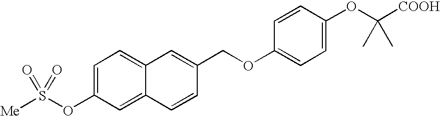 | 2-methyl-2-[4-{6- methanesulfonyloxynapth-2- ylmethoxy}phenoxy]propanoic acid |
| 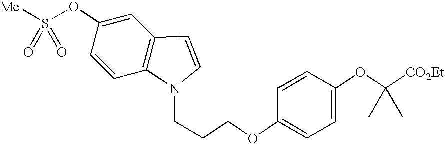 | Ethyl 2-methyl-2-[4-{3-(5- methanesulfonyloxyindol-1-yl) propyloxy}phenoxy]propanoate |
| 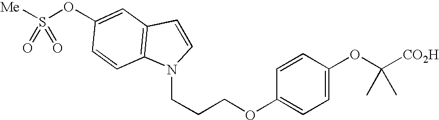 | 2-methyl-2-[4-{3-(5- methanesulfonyloxyindol-1-yl) propyloxy}phenoxy]propanoic acid |

45. A compound of formula (Id) according to claim 28 which is selected from:

| Structure | IUPAC Name |
|---|---|
| 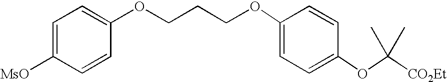 | Ethyl 2-methyl-2-[4-{3-(4- methanesulfonyloxyphenoxy) propyloxy}phenoxy]propanoate |
| 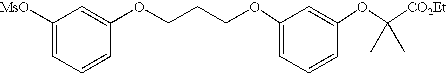 | Ethyl 2-methyl-2-[3-{3-(3- methanesulfonyloxyphenoxy) propyloxy}phenoxy]propanoate |
| 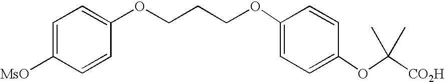 | 2-Methyl-2-[4-{3-(4- methanesulfonyloxyphenoxy) propyloxy}phenoxy]propanoic acid |
| 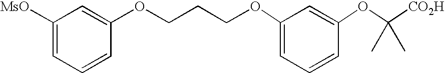 | 2-Methyl-2-[3-{3-(3- methanesulfonyloxyphenoxy) propyloxy}phenoxy]propanoic acid |
| 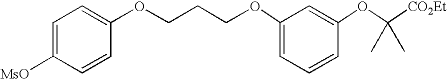 | Ethyl 2-methyl-2-[3-{3-(4- methanesulfonyloxyphenoxy) propyloxy}phenoxy]propanoate |
| 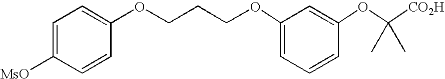 | 2-Methyl-2-[3-{3-(4- methanesulfonyloxyphenoxy) propyloxy}phenoxy]propanoic acid |

46. A compound of formula (Id) according to claim 28 which is selected from:

| Structure | IUPAC Name |
|---|---|
| 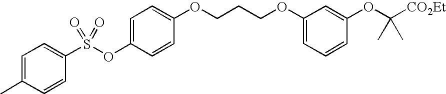 | Ethyl 2-methyl-2-[3-{3-(4-(para- toluenesulfonyloxy)phenoxy) propyloxy}phenoxy]propanoate |
| 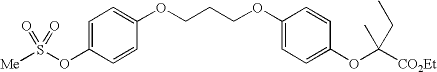 | Ethyl 2-methyl-2-[4-{3-(4- methanesulfonyloxyphenoxy) propyloxy}phenoxy]butanoate |
| 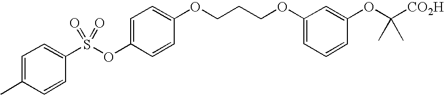 | 2-methyl-2-[3-{3-(4-(para- toluenesulfonyloxy)phenoxy) propyloxy}phenoxy]propanoic acid |
| 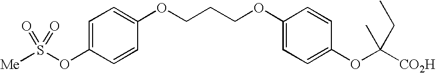 | 2-Methyl-2-[4-{3-(4- methanesulfonyloxyphenoxy) propyloxy}phenoxy]butanoic acid |

47. A compound of formula (Id) according to claim 28 which is selected from:

| Structure | IUPAC Name |
|---|---|
| 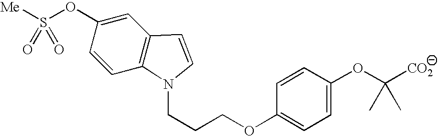 | 2-methyl-2-[4-{3-(5- methanesulfonyloxyindol-1-yl) propyloxyl phenoxy]propanoic acid Arginine salt |
| 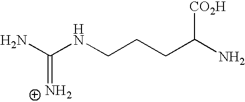 | |
| 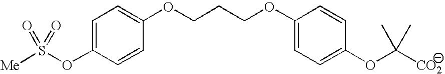 | 2-Methyl-2-[4-{3-(4- methanesulfonyloxyphenoxy) propyloxy}phenoxy]propanoic acid Arginine salt |
| 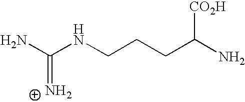 | |
| 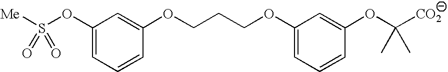 | 2-Methyl-2-[3-{3-(3- methanesulfonyloxyphenoxy) propyloxy}phenoxy]propanoic acid Arginine salt |
| 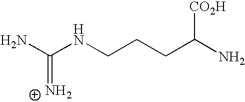 | |
| 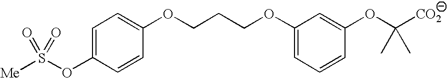 | 2-Methyl-2-[3-{3-(4- methanesulfonyloxyphenoxy) propyloxy}phenoxy]propanoic acid Arginine salt |
| 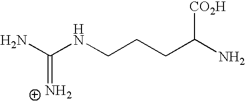 | |
| 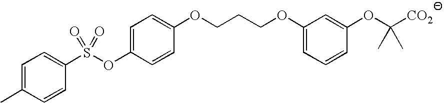 | 2-Methyl-2-[3-{3-(4-(para- toluenesulfonyloxy)phenoxy) propyloxy}phenoxy]propanoic acid, arginine salt |
| 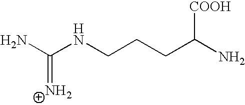 | |
| 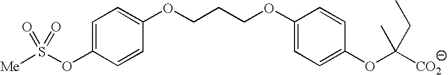 | 2-Methyl-2-[4-{3-(4- methanesulfonyloxyphenoxy) propyloxy}phenoxy]butanoic acid, arginine salt |
| 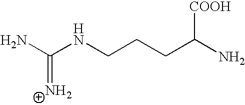 | |

48. The compound of formula (Ie) according to claim 1, which is 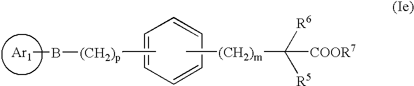
wherein all of the symbols are as defined above.

49. The compound of claim 48, wherein Ar₁ is substituted with —OSO₂R³, and R³ is alkyl or aryl.

50. The compound of formula (If) according to claim 1, which is, 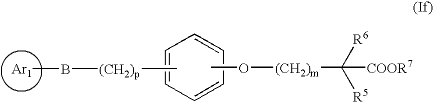
wherein all of the symbols are as defined above.

51. The compound of claim 15, wherein Ar₁ is substituted with —OSO₂R³, where R³ is selected from optionally substituted groups selected from alkyl or aryl.

52. The compound of formula (Ie) as claimed in claim 1 is selected from:

| Structure | IUPAC Name |
|---|---|
| 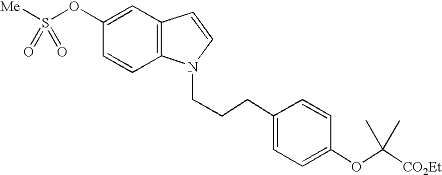 | Ethyl 2-methyl-2- [4-{3-(5-methanesulfonyloxyindol-1-yl) propyl} phenoxy] propanoate |
| 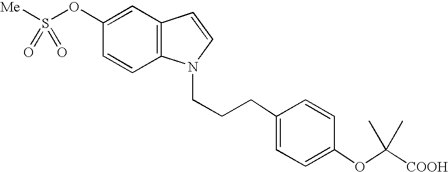 | 2-methyl-2- [4-{3-(5-methanesulfonyloxyindol-1-yl) propyl}phenoxy] propanoic acid |
| 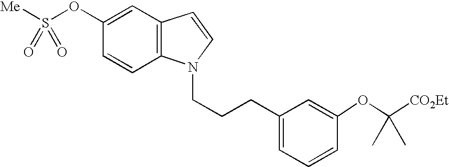 | Ethyl 2-methyl-2- [3-{3-(5-methanesulfonyloxyindol-1-yl) propyl} phenoxy] propanoate |
| 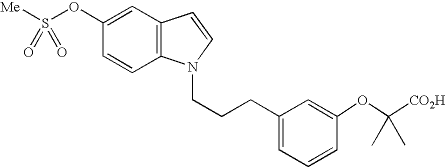 | 2-methyl-2- [3-{3-(5-methanesulfonyloxyindol-1-yl) propyl}phenoxy] propanoic acid |
| 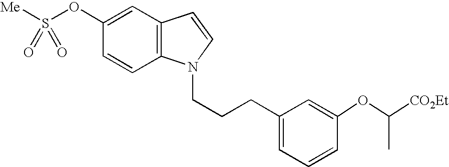 | Ethyl 2-[3-{3-(5-methanesulfonyloxyindol-1-yl) propyl}phenoxy] propanoate |
| 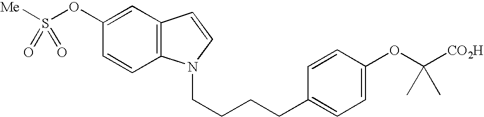 | 2-Methyl-2-[4-{4-(5-methanesulfonyloxyindol- 1yl)butyl}phenoxy]propanoic acid |
| 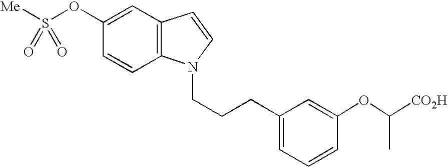 | 2-[3-{3-(5-Methanesulfonyloxyindol-1- yl)propyl}phenoxy]propanoic acid |
| 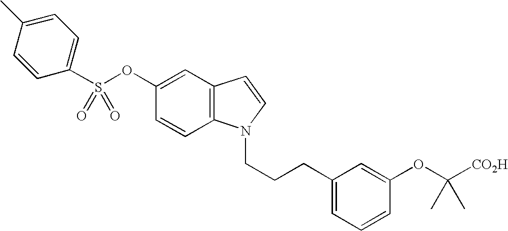 | 2-Methyl-2-[3-{3-(5-(para-toluenesulfonyloxy)indol-1- yl)propyl}phenoxy} propanoic acid |
| 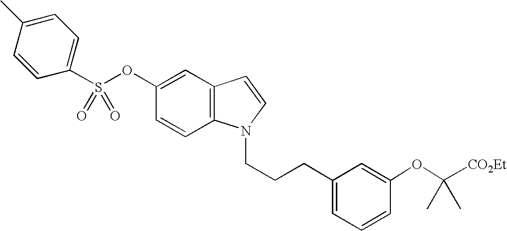 | Ethyl 2-methyl-2-[3-{3-(5-(para-toluenesulfonyloxy)indol-1- yl)propyl}phenoxy] propanoate |

53. The compound of formula (Ie) as claimed in claim 1 is selected from:

| Structure | IUPAC Name |
|---|---|
| 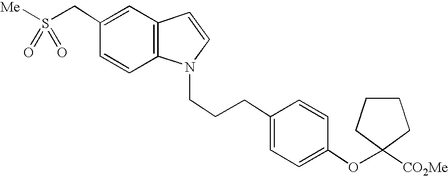 | 1-[4-{3-(5-methanesulfonyloxyindol-1- yl)propyl}phenoxy]cyclopentane-1-carboxylic acid, methyl ester |
| 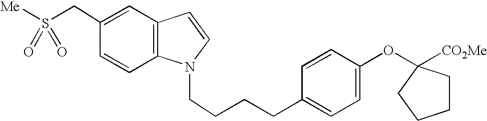 | 1-[4-{4-(5-methanesulfonyloxyindol-1- yl)butyl}phenoxy]cyclopentane-1-carboxylic acid, methyl ester |
| 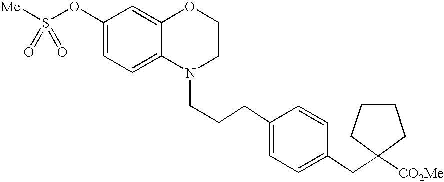 | 1-[4-{3-(7-Methanesulfonyloxy-3,4-dihydro-2H-bezo [b] [1,4]oxazin-4-yl)propyl}phenoxy]cyclopentane-1-carboxylic acid, methyl ester |
| 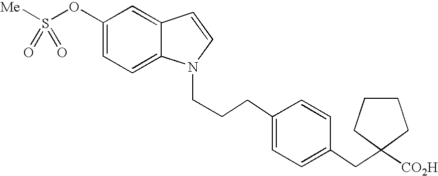 | 1-[4-{3-(5-Methanesulfonyloxyindol-1- yl)propyl}phenoxy]cyclopentane-1-carboxylic acid |
| 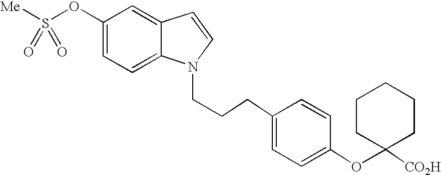 | 1-[4-{3-(5-methanesulfonyloxyindol-1- yl)propyl}phenoxy]cyclohexane-1-carboxylic acid |
| 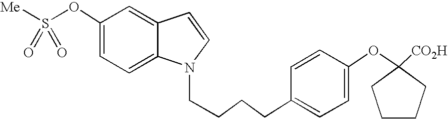 | 1-[4-{4-(5-methanesulfonyloxyindol-1- yl)butyl}phenoxy]cyclopentane-1-carboxylic acid |
| 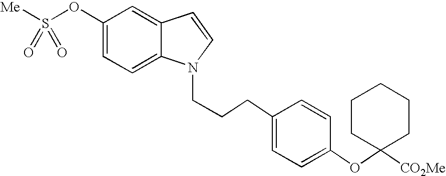 | 1-[4-{3-(5-Methanesulfonyloxyindol-1- yl)propyl}phenoxy]cyclohexane-1-carboxylic acid, methyl ester |

54. The compound of formula (Ie) as claimed in claim 1 is selected from:

| Structure | IUPAC Name |
|---|---|
| 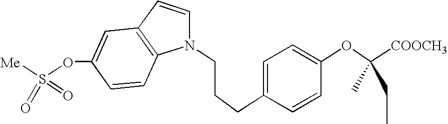 | (+) Methyl (R)-2-methyl-2-[4-{3-(5-methanesulfonyloxyindol-1- yl)propyl}phenoxy] butanoate |
| 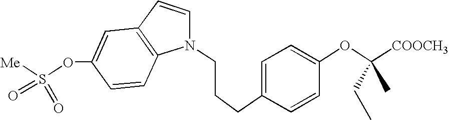 | (−) Methyl (S)-2-methyl-2-[4-{3-(5-methanesulfonyloxyindol-1- yl)propyl}phenoxy] butanoate |
| 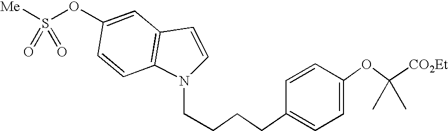 | Ethyl 2-methyl-2-[4-{4-(5-methanesulfonyloxyindol-1- yl)butyl}phenoxy]propanoate |
| 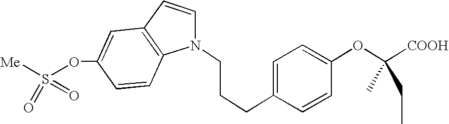 | (R)- (+)-2-methyl-2-[4-{3-(5-methanesulfonyloxyindol-1-yl) propyl}phenoxy] butanoic acid |
| 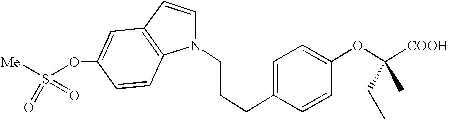 | (S)- (−)-2-methyl-2-[4-{3-(5-methanesulfonyloxyindol-1-yl)propyl}phenoxy]butanoic acid |

55. The compound of formula (Ie) as claimed in claim 1 is selected from:

| Structure | IUPAC Name |
|---|---|
| 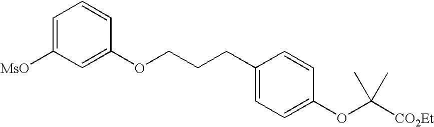 | Ethyl 2-methyl-2-[4-{3-(3-methanesulfonyloxyphenoxy) propyl} phenoxy] propanoate |
| 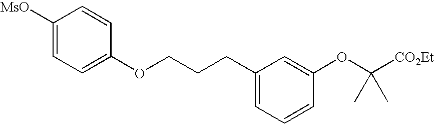 | Ethyl 2-methyl-2-[3-{3-(4-methanesulfonyloxyphenoxy) propyl} phenoxy] propanoate |
| 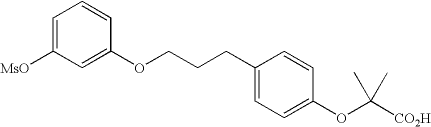 | 2-Methyl-2-[4-{3-(3-methanesulfonyloxyphenoxy) propyl}phenoxy] propanoic acid |
| 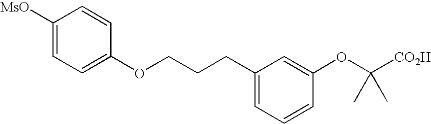 | 2-Methyl-2-[3-{3-(4-methanesulfonyloxyphenoxy) propyl}phenoxy] propanoic acid |
| 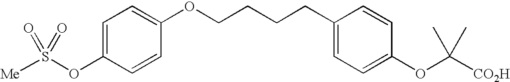 | 2-Methyl-2-[4-{4-(4-methanesulfonyloxyphenoxy) butyl}phenoxy]propanoic acid |
| 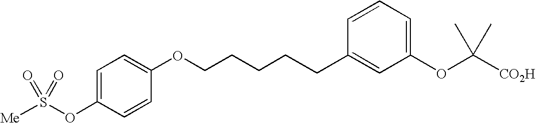 | 2-Methyl-2-[3-{5-(4- methanesulfonyloxyphenoxy)pentyl}phenoxy]propanoic acid |

56. The compound of formula (Ie) as claimed in claim 1 is selected from:

| Structure | IUPAC Name |
|---|---|
| 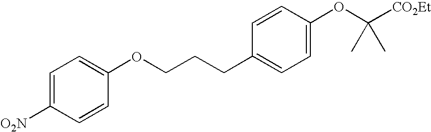 | Ethyl 2-methyl-2-[3-{5-(4- nitrophenoxy)propyl}phenoxy]propanoate |
| 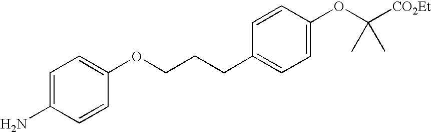 | Ethyl 2-methyl-2-[3-{5-(4- aminophenoxy)propyl}phenoxy]propanoate |
| 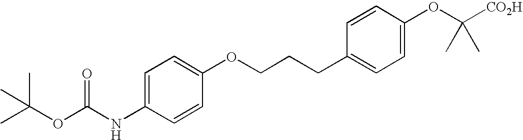 | 2-Methyl-2-[4-{3-(4-(tert- butyloxycarbonylamino)phenoxy)propyl}phenoxy]propanoic acid |
| 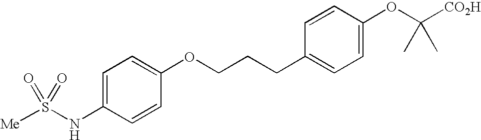 | 2-Methyl-2-[4-{3-(4- (methanesulfonylamino)phenoxy)propyl}phenoxy]propanoic acid |
| 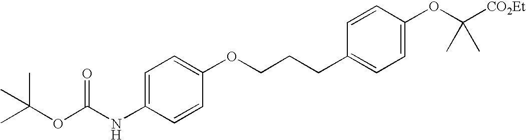 | Ethyl 2-methyl-2-[4-{3-(4-(tert- butyloxycarbonylamino)phenoxy)propyl}phenoxy]propanoate |
| 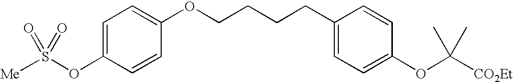 | Ethyl 2-methyl-2-[4-{4-(4- methanesulfonyloxyphenoxy)butyl}phenoxy]propanoate |
| 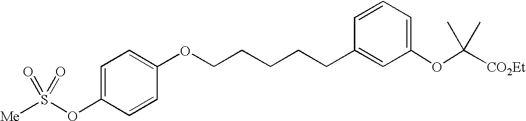 | Ethyl 2-methyl-2-[3-{5-(4- methanesulfonyloxyphenoxy)pentyl}phenoxy]propanoate |
| 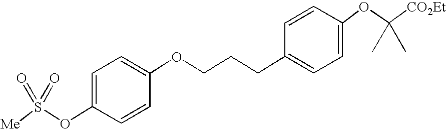 | Ethyl 2-methyl-2-[4-{3-(4- (methanesulfonylamino)phenoxy)propyl}phenoxy]propanoate |

57. The compound of formula (Ie) as claimed in claim 1 is selected from:

| Structure | IUPAC Name |
|---|---|
| 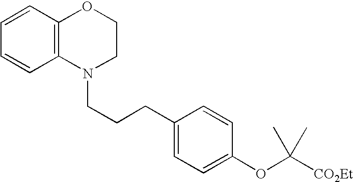 | Ethyl 2-methyl-2- [4-{3-(3, 4-dihydro-2H-bezo [b] [1, 4] Oxazin-4-yl) propyl} phenoxy] propanoate |
| 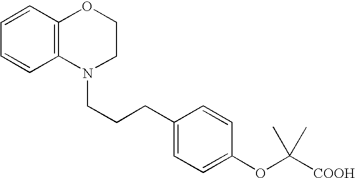 | 2-methyl-2- [4-{3-(3, 4-dihydro-2H-bezo [b] [1, 4] Oxazin-4-yl) propyl}phenoxy] propanoic acid |
| 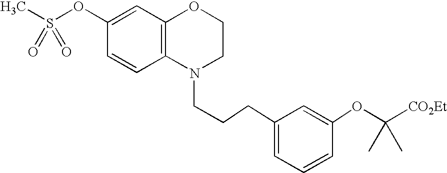 | Ethyl-2-methyl-2-[3-{3-(7-Methanesulfonyloxy-3, 4-dihydro-2H-bezo [b] [1, 4] oxazin-4-yl) propyl{ phenoxy] propanoate. |
| 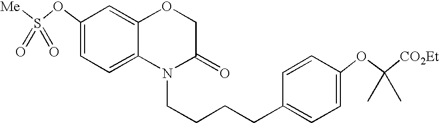 | Ethyl 2-methyl-2-[4-{4-(7-methanesulfonyloxy-3, 4-dihydro-2H-bezo [b] [1, 4] oxazin-3-on-4-yl)butyl}phenoxy]propanoate |
| 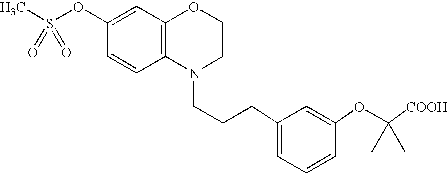 | 2-Methyl-2-[3-{3-(7-Methanesulfonyloxy-3, 4-dihydro-2H-bezo [b] [1, 4]oxazin-4-yl) propyl} phenoxy] propanoic acid |
| 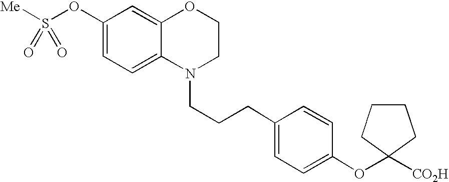 | 1-[4-{3-(7-Methanesulfonyloxy-3, 4-dihydro-2H-bezo [b] [1, 4] oxazin-4- yl)propyl}phenoxy] cyclopentane-1-carboxylic acid |
| 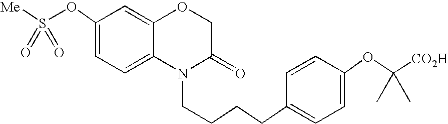 | 2-Methyl-2-[4-{4-(7-methanesulfonyloxy-3, 4-dihydro-2H-bezo [b] [1, 4]oxazin-3-on-4-yl)butyl}phenoxy]propanoic acid |

58. The compound of formula (Ie) as claimed in claim 1 is selected from:

| Structure | IUPAC Name |
|---|---|
| 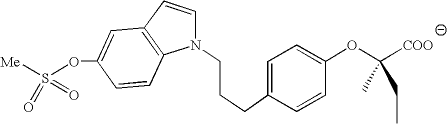 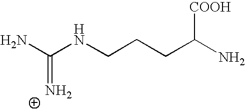 | (R)- (+)-2-methyl-2-[4-{3-(5-methanesulfonyloxyindol-1-yl) propyl} phenoxy] butanoic acid, Arginine salt |
| 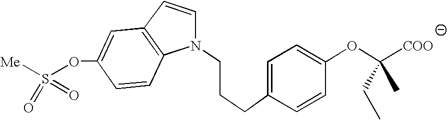 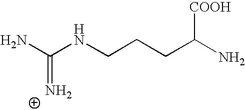 | (S)- (−)-2-methyl-2-[4-{3-(5-methanesulfonyloxyindol-1-yl) propyl} phenoxy] butanoic acid, Arginine salt |
| 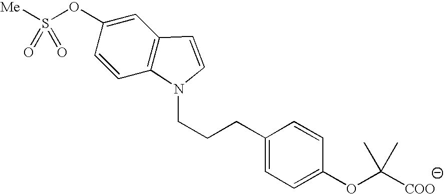 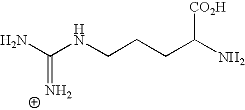 | 2-methyl-2- [4-{3-(5-methanesulfonyloxyindol-1-yl) propyl}phenoxy] propanoic acid Arginine salt |
| 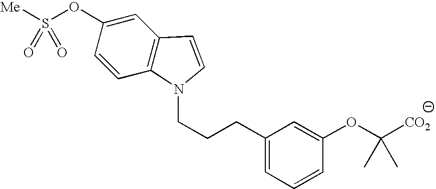 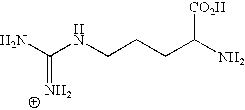 | 2-methyl-2- [3-{3-(5-methanesulfonyloxyindol-1-yl) propyl}phenoxy] propanoic acid Arginine salt |
| 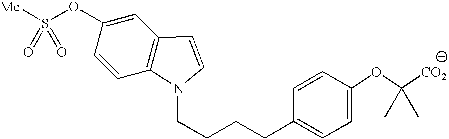 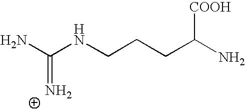 | 2-Methyl-2-[4-{4-(5-methane sulfonyloxyindol- 1yl)butyl}phenoxy]propanoic acid, arginine salt |
| 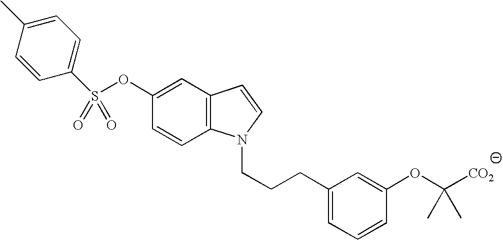 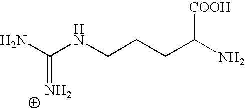 | 2-Methyl-2-[3-{3-(5-(para-toluenesulfonyloxy)indol-1-yl)propyl}phenoxy] propanoic acid, arginine salt |
| 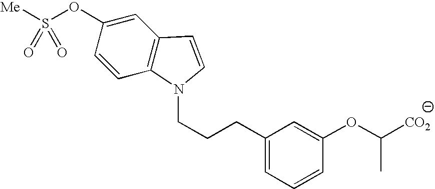 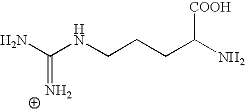 | 2-[3-{3-(5-Methanesulfonyloxyindol-1- yl)propyl}phenoxy]propanoic acid, arginine |
| 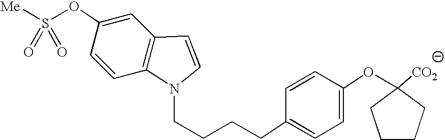 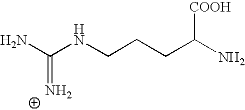 | 1-[4-{4-(5-methanesulfonyloxyindol-1- yl)butyl}phenoxy]cyclopentane-1-carboxylic acid, arginine salt |

59. The compound of formula (Ie) as claimed in claim 1 is selected from:

| Structure | IUPAC Name |
|---|---|
| 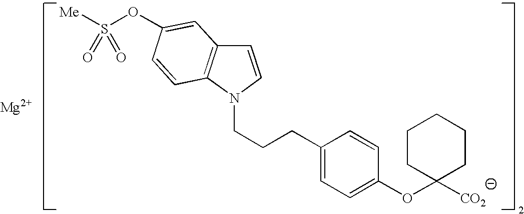 | 1-[4-{3-(5-methanesulfonyloxyindol-1- yl)propyl}phenoxy]cyclohexane-1-carboxylic acid, magnesium salt |
| 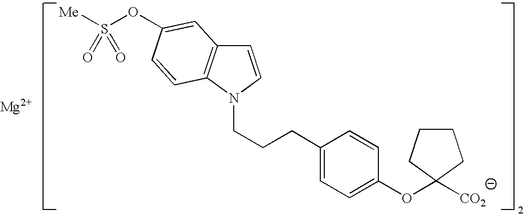 | 1-[4-{3-(5-Methanesulfonyloxyindol-1- yl)propyl}phenoxy]cyclopentane-1-carboxylic acid, magnesium salt |
| 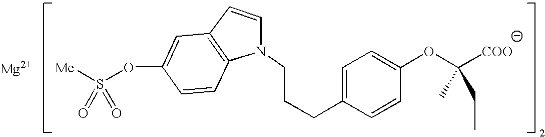 | (racemic) Methyl-2-methyl-2-[4-{3-(5- methanesulfonyloxyindol-1-yl) propyl} phenoxy] butanoic acid Magnesium salt |

60. The compound of formula (Ie) as claimed in claim 1 is selected from:

| Structure | IUPAC Name |
|---|---|
| 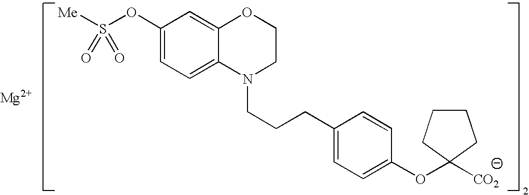 | 1-[4-{3-(7-Methanesulfonyloxy-3, 4-dihydro-2H-bezo [b] [1, 4] oxazin-4-yl)propyl}phenoxy]cyclopentane-1-carboxylic acid, magnesium salt |
| 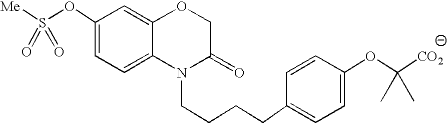 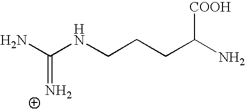 | 2-Methyl-2-[4-{4-(7-methanesulfonyloxy-3, 4-dihydro-2H- bezo [b] [1, 4] oxazin-3-on-4-yl)butyl}phenoxy]propanoic acid, Arginine salt |
| 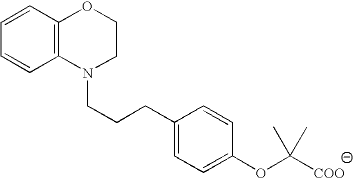 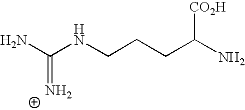 | 2-methyl-2- [4-{3-(3,4-dihydro-2H-bezo [b][1,4] Oxazin-4-yl) propyl} phenoxy] propanoic acid Arginine salt |
| 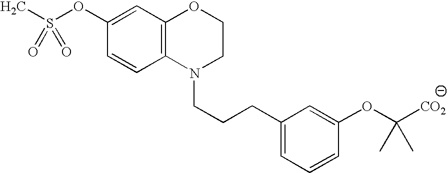 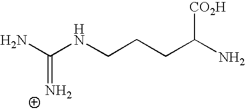 | 2-Methyl-2-[3-{3-(7-Methanesulfonyloxy-3, 4-dihydro-2H- bezo [b] [1, 4] oxazin-4-yl) propyl} phenoxy] propanoic acid, Arginine salt |

61. The compound of formula (Ie) as claimed in claim 1 is selected from:

| Structure | IUPAC Name |
|---|---|
| 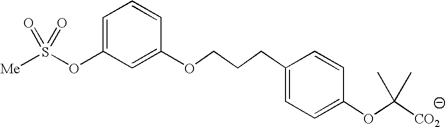 | 2-Methyl-2-[4-{3-(3-methanesulfonyloxyphenoxy) propyl}phenoxy] propanoic acid Arginine salt |
| 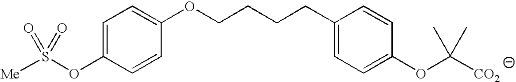 | 2-Methyl-2-[4-{4-(4- methanesulfonyloxyphenoxy)butyl}phenoxy]propanoic acid, arginine salt |
| 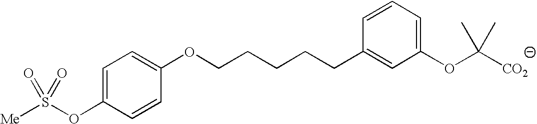 | 2-Methyl-2-[3-{5-(4- methanesulfonyloxyphenoxy)pentyl}phenoxy]propanoic acid, arginine salt |

62. A process for the preparation of compound of formula (I), 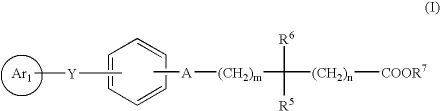
wherein Ar₁ represents 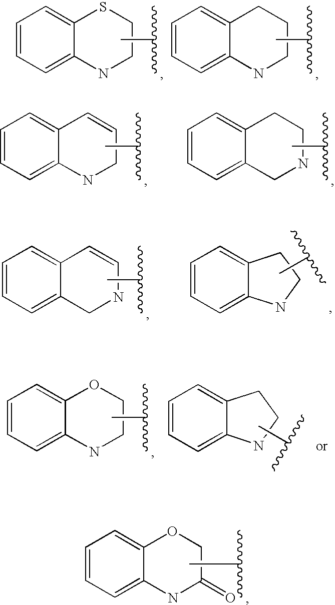
and all other symbols are as defined above, which process comprises, reacting compound of formula (8) 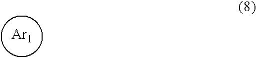
wherein Ar₁ represents 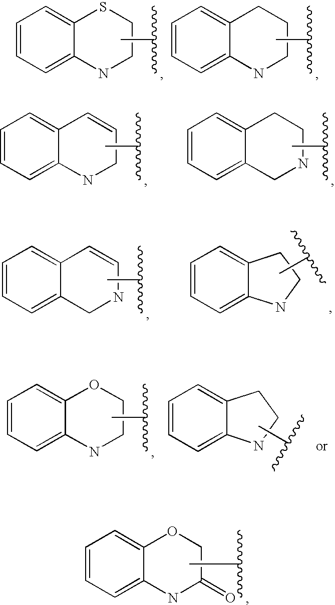
with a compound of formula (9) 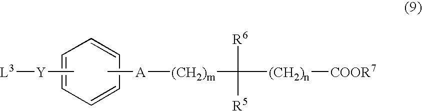
where L³ represents a leaving group selected from halo or mesyloxy, and all other symbols have the meaning as described above.

63. A pharmaceutical composition, which comprises a compound of formula (I) 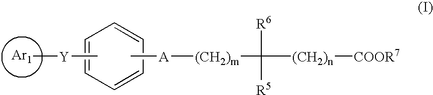
as defined in claim 1 and a pharmaceutically acceptable carrier, diluent, excipient or solvate.

64. The pharmaceutical composition of claim 63, wherein the compound is as claimed in claims 30.

65. The pharmaceutical composition of claim 63, wherein the compound is as claimed in claims 38.

66. The pharmaceutical composition of claim 63, wherein the compound is as claimed in claims 41.

67. The pharmaceutical composition of claim 63, wherein the compound is as claimed in claims 43.

68. The pharmaceutical composition of claim 63, wherein the compound is as claimed in claims 49.

69. The pharmaceutical composition of claim 63, wherein the compound is as claimed in claims 51.

70. A pharmaceutical composition as claimed in claim 63, in the form of a tablet, capsule, powder, syrup, solution or suspension.

71. A method for treating and/or preventing dyslipidemia comprising administering a compound of formula (I) as defined in claim 1 or a pharmaceutical composition according to claim 63 to a patient in need thereof.

72. A method for treating and/or preventing diabetes caused by insulin resistance or impaired glucose tolerance comprising administering a compound of formula (I) as defined in claim 1 or a pharmaceutical composition according to claim 63 to a patient in need thereof.

73. Use of a compound of formula (I) as defined in claim 1 or a pharmaceutical composition according to claim 63 for treating and/or preventing dyslipidemia.

74. Use of a compound of formula (I) as defined in claim 1 or a pharmaceutical composition according to claim 63 for treating and/or preventing diabetes caused by insulin resistance or impaired glucose tolerance.

75. A medicine for treating and/or preventing diabetes caused dyslipidemia comprising administering a compound of formula (I) as defined in claim 1 or a pharmaceutical composition according to claim 63 to a patient in need thereof.

76. A medicine for treating and/or preventing diabetes caused by insulin resistance or impaired glucose tolerance comprising administering a compound of formula (I) as defined in claim 1 or a pharmaceutical composition according to claim 63 to a patient in need thereof.
